(19) United States
(12) Patent Application Publication
(10) Pub. No.: US 2026/0317301 A1
(43) Pub. Date: Oct. 15, 2026

(54) LACTAM PESTICIDAL COMPOUNDS

(71) Applicant: BASF SE, Ludwigshafen (DE)

(72) Inventors: Matthew Charles Linford Wakeham, Ludwigshafen (DE); Christian Defieber, Ludwigshafen (DE); Pulakesh Maity, Navi Mumbai (IN); Karsten Koerber, Ludwigshafen (DE); Rupsha Chaudhuri, Navi Mumbai (IN); Ashokkumar Adisechan, Navi Mumbai (IN); Christian Winter, Ludwigshafen (DE); Kishor Handore, Navi Mumbai (IN)

(21) Appl. No.: 19/485,703

(22) PCT Filed: May 23, 2024
(86) PCT No.: PCT/EP2024/064183

§ 371 Date: Nov. 19, 2025

(30) Foreign Application Priority Data
May 25, 2023 (EP) 23175523.2
Jul. 4, 2023 (EP) 23183305.4

(51) Int. Cl. C07D 403/04; A01N 43/56; A01N 43/58; A01N 43/60; A01N 43/653; A01N 43/707; A01P 5/00; A01P 7/02; A01P 7/04; A01P 17/00; C07D 401/14

(52) U.S. Cl. CPC C07D 403/04; A01N 43/56; A01N 43/58; A01N 43/60; A01N 43/653; A01N 43/707; A01P 5/00; A01P 7/02; A01P 7/04; A01P 17/00; C07D 401/14

(57) ABSTRACT

The present invention relates to the compounds of formula (I), and the N-oxides, stereoisomers, tautomers and agriculturally or veterinarily acceptable salts thereof wherein the variables are defined according to the description, formula (I). The compounds of formula (I), as well as the N-oxides, stereoisomers, tautomers and agriculturally or veterinarily acceptable salts thereof, are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

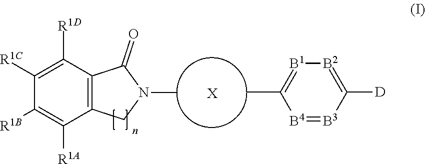

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

Carbamoylated and thiocarbamoylated oxime derivatives are known for pesticidal use, for example, in patent publications WO 2016/156076, semi-carbazones and thiosemicarbazones derivatives are known for pesticidal use in patent publication WO 2016/116445, WO2021/013561.

Due to the ability of target pests to develop resistance to pesticidally-active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes.

Nevertheless, there remains a need for highly effective and versatile agents for combating invertebrate pests. It is therefore an object of the invention to provide compounds having a good pesticidal activity and showing a broad activity spectrum against a large number of different invertebrate pests, especially against difficult to control pests, such as insects.

It has been found that these objects can be achieved by compounds of formula I, as depicted and defined below, including their stereoisomers, their salts, in particular their agriculturally or veterinarily acceptable salts, their tautomers and their N-oxides.

In a first aspect, the present invention relates to compounds of formula I

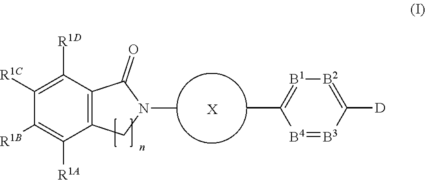

wherein
R^{1A}, R^{1B}, R^{1C} and R^{1D} are, identical or different, H, halogen, SF₅, OH, CN, C₁-C₆-alkyl, C₁-C₆-alkoxy, C₃-C₆-cycloalkyl, C₃-C₆-cycloalkoxy wherein the alkyl, alkoxy, cycloalkyl, and cycloalkoxy moieties are unsubstituted or substituted with halogen or CN;
NR⁸R⁹, C═O(NR⁸R⁹), S(═O)ₘ(NR⁸R⁹), or NHS(═O)ₘR⁸
m is 1 or 2
n is 1, 2 or 3;
X is defined by either one of the following two rings X1 or X2

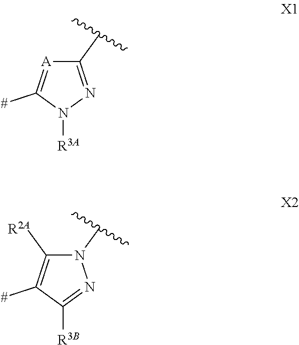

wherein
# denotes connection to the lactam moiety;
A is N or CR^{2A};
R^{2A} is H, C₁-C₆-alkyl, C₃-C₆-cycloalkyl, C₁-C₆-alkoxy, wherein the alkyl, alkoxy and cycloalkyl moieties are unsubstituted or substituted with halogen or CN; halogen, CN, OR⁶, or NR⁶R⁷
R^{3A} is H, C₁-C₆-alkyl, C₃-C₆-cycloalkyl, C₁-C₆-alkoxy, halogen, or CN wherein the alkyl, alkoxy, and cycloalkyl moieties are unsubstituted or substituted with halogen, CN, or C₁-C₆-alkoxy;
R^{3B} is H, C₁-C₆-alkyl, C₃-C₆-cycloalkyl, C₁-C₆-alkoxy, wherein the alkyl, alkoxy, and cycloalkyl moieties are unsubstituted or substituted with halogen or CN;
halogen, CN, OR⁶, or NR⁶R⁷,
B¹ is N or CR^{B1};
B² is N or CR^{B2};
B³ is N or CR^{B3};
B⁴ is CR^{B4};
R^{B1}, R^{B2}, R^{B3}, and RB⁴ are, identical or different, H, halogen, OH, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
D is the moiety DA, DB, DC, DD, DE, or DF

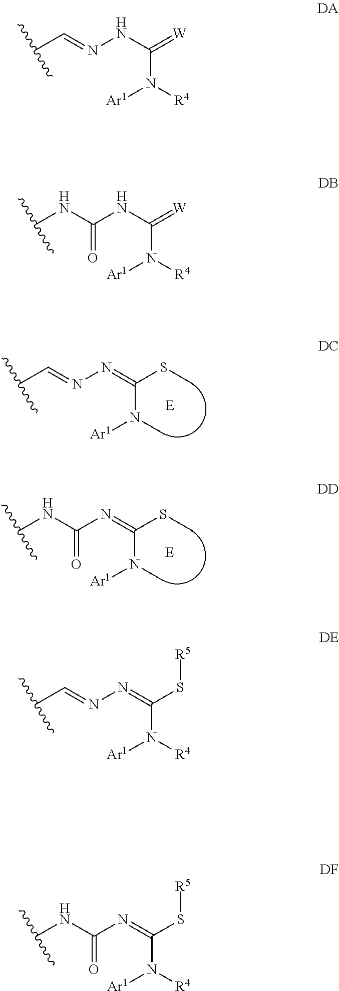

wherein
W is S or O;
R⁴ is H, C₁-C₆-alkyl, or C₃-C₆-cycloalkyl, wherein the alkyl and cycloalkyl moieties are unsubstituted or substituted with halogen or CN;
R⁵ is H, C₁-C₆-alkyl, or C₃-C₆-cycloalkyl, wherein the alkyl and cycloalkyl moieties are unsubstituted or substituted with —O—(C═O)—C₁-C₆-alkoxy, —O—(C═O)—C₁-C₆-alkyl or CN;
E is a 5- or 6-membered carbocyclic group, wherein 1 or 2 CH₂ moieties of the carbocyclic group may be replaced by a carbonyl group, O, or S, wherein the carbocyclic group is unsubstituted or substituted with R¹⁰;
R⁶ and R⁷ are, identical or different, H, C₁-C₆-alkyl, C₃-C₆-cycloalkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, cycloalkyl, phenyl and heteroaryl moieties are unsubstituted or substituted with halogen, CN, C₁-C₆-alkyl or C₁-C₆-alkoxy;
Ar¹ is phenyl or 5- or 6-membered heteroaryl, which are unsubstituted or substituted with R^{Ar1}, wherein
R^{Ar1} is halogen, SF₅, OH, CN, C₁-C₆-alkyl, C₁-C₆-alkoxy, C₃-C₆-cycloalkyl, C₃-C₆-cycloalkoxy, or C₁-C₆-alkyl-O—C₁-C₆-alkyl, wherein the alkyl, alkoxy, cycloalkyl, and cycloalkoxy moieties are unsubstituted or substituted with halogen or CN;
NR⁸R⁹, C═O(NR⁸R⁹), S(═O)ₘ(NR⁸R⁹), NHS(═O)ₘR⁸;
R⁸ and R⁹ are, identical or different, H, C₁-C₆-alkyl, C₃-C₆-cycloalkyl wherein the alkyl, and cycloalkyl moieties are unsubstituted or substituted with halogen;
R¹⁰ is halogen, C₁-C₆-alkyl, or C₁-C₆-alkoxy;
and the N-oxides, stereoisomers, tautomers and agriculturally or veterinarily acceptable salts thereof.

Moreover, the present invention also relates to processes and intermediates for preparing compounds of formula I and to active compound combinations comprising them. Moreover, the present invention relates to agricultural or veterinary compositions comprising the compounds of formula I, and to the use of the compounds of formula I or compositions comprising them for combating or controlling invertebrate pests and/or for protecting crops, plants, plant propagation material and/or growing plants from attack and/or infestation by invertebrate pests. The present invention also relates to methods of applying the compounds of formula I. The present invention also relates to method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a pesticidally effective amount of at least one compound of formula (I) as defined above or a composition comprising at least one compound of formula (I);

Furthermore, the present invention relates to seed comprising compounds of formula I. Wherein the compounds of formula I includes N-oxides, stereoisomers, tautomers and agriculturally or veterinarily acceptable salts thereof.

With due modification of the starting compounds, the compounds of formula I can be prepared by procedures as given in below schemes.

The compounds of the formula (I) can be prepared by methods of organic chemistry, e.g, by the methods described herein after in schemes 1 to 16 in the synthesis description of the examples. In the schemes 1 to 16 the radicals Hal is halogen and R^{1A}, R^{1B}, R^{1C}, R^{1D}, m, n, X, Y, Z, X′″, A, L, D, DX′, DX″, R^{2A}, R^{3A}, R^{3B}, B¹, B², B³, B⁴, R⁴, RB¹, RB², RB³, RB⁴, R⁴, R⁵, E, R⁶, R⁷, W, Ar¹, R^{Ar1}, R⁸, R⁹ and R¹⁰ are as defined above for formula (I), unless otherwise specified.

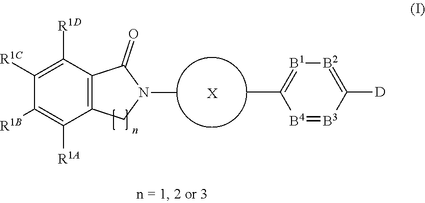

wherein X is

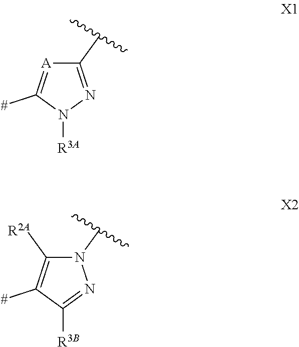

and D is

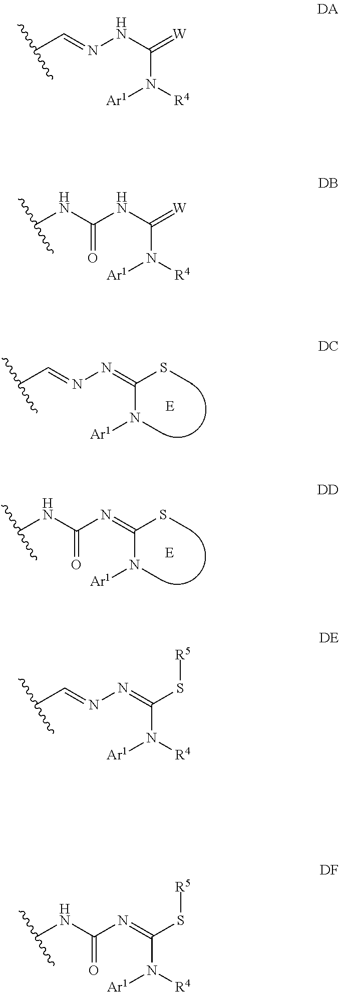

Compounds of formula (I), wherein D is DA, DC or DE are the compounds of formula (1-2) can be prepared analogously to the methods described in WO 2020/083733 or methods described in Scheme 4 and Scheme 6.

Compounds of formula (I), wherein D is DB, DD or DF are the compounds of formula (I-1) can be prepared analogously to the methods described in WO 2021/011722 or methods described in Scheme 1, Scheme 2, Scheme 4 and Scheme 5.

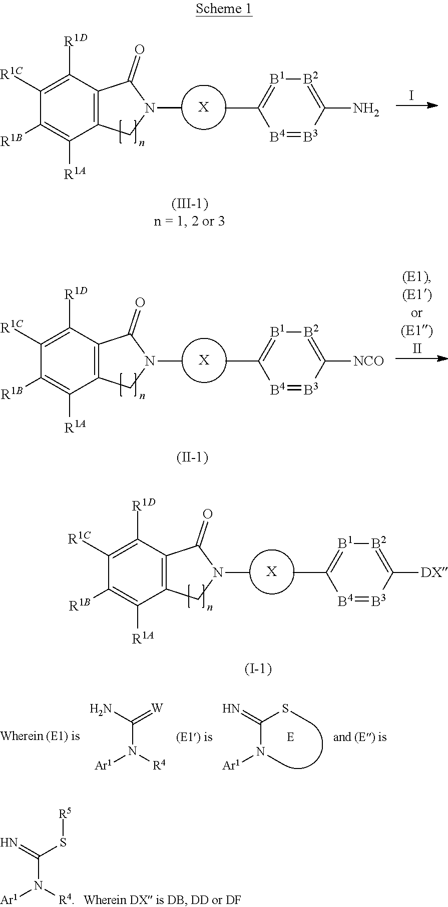

In one embodiment of Scheme 1, compounds of formula (II-1) are reacted directly with a compound of formula (E1), (E1′) or (E1″) in the presence of an inorganic base to form a compound of formula (1-1). An isocyanate compound of formula (II-1) can be generated in situ from either an amine of the formula (III-1) by using one of the common reagents such as phosgene, diphosgene, triphosgene or carbonyldiimidazole, as in step (I), in a mixed solvent system and in the presence of a base as described in March's Advanced Organic Chemistry 6^{th} edition, Michael B. Smith and Jerry March or by the methods described in WO 2021/011722 or WO 2014/204622.

Compounds of formula (III-1) can also be prepared analogously to the methods described in WO 2021/011722, WO 2014/204622 or methods described in Scheme 8.

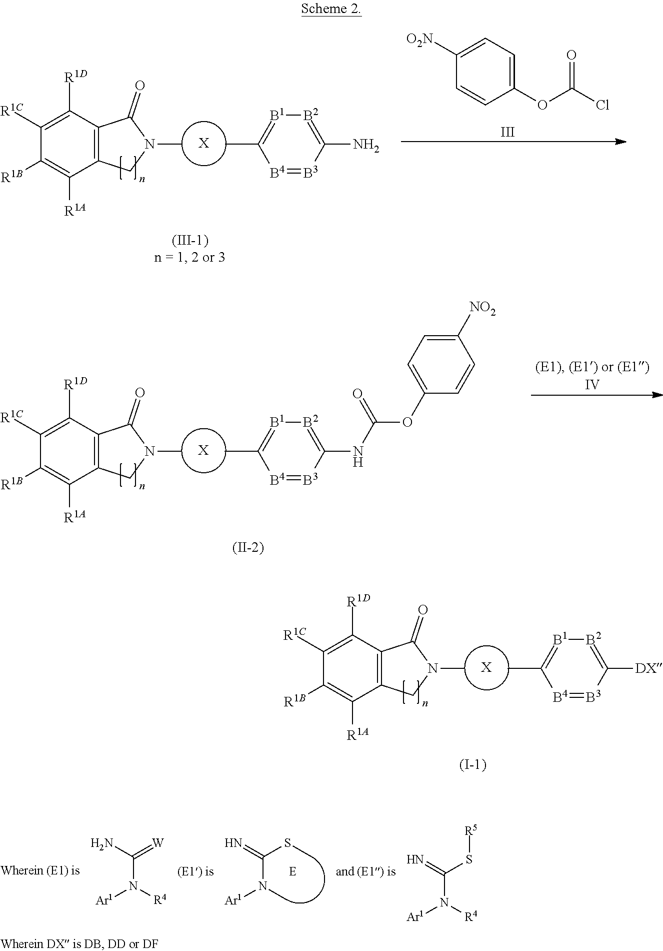

According to the method depicted in scheme 2, amine of formula (III-1) can be treated with an activating agent such as 4-nitrophenyl chloroformate in the presence of a polar aprotic solvent preferably tetrahydrofuran to generate an activated carbamate intermediate (II-2), which in turn is reacted with the compound of formula (E1), (E1′) or (E1″) in the presence of organic base such as DIPEA to form compounds of formula (I-1).

Compounds of formula (E1) (E1′) or (E1″) can be prepared analogously to the methods described in WO 2021/011722, J. of Med. Chem. 2010, 53(10), 4198-4211, Synthesis 1988, 1998 (6), 460-466 or can be prepared analogously to the methods described in Scheme 3. Compounds of formula (III-1) can also be prepared analogously to the methods described in WO 2021/011722, WO 2014/204622 or methods described in Scheme 8.

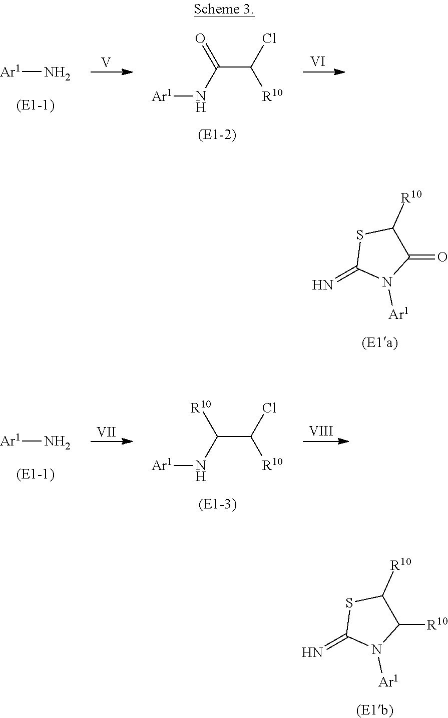

In the above reactions, compounds of formula (E1-1) can be converted into cyclized analogs of formulae (E1′a). Compounds of formula (E1-2) can be prepared by treatment of compounds of formula (E1-1) with unsubstituted or mono- or di-substituted 2-chloroacetylchloride in two steps as depicted in J. Med. Chem. 2010, 53(10), 4198-4211. Compounds of formula (E1-3) can be prepared by treatment of compounds of formula (E1-1) with unsubstituted or mono- or di-substituted 2-chloroactaldehyde in two steps as mentioned in J. Het. Chem. 2006, 43(6), 1523-1531. Compounds of formula (E1′b) can be prepared by treatment of compounds of formula (E1-3) with potassium thiocyanate in presence of inorganic bases such as cesium carbonate in an aprotic solvent such as acetone.

Compounds containing 6-membered rings substituted or unsubstituted with R¹⁰ can also be prepared analogously by the methods described in J. Med. Chem. 2010, 53(10), 4198-4211, and J. Het. Chem. 2006, 43(6), 1523-1531.

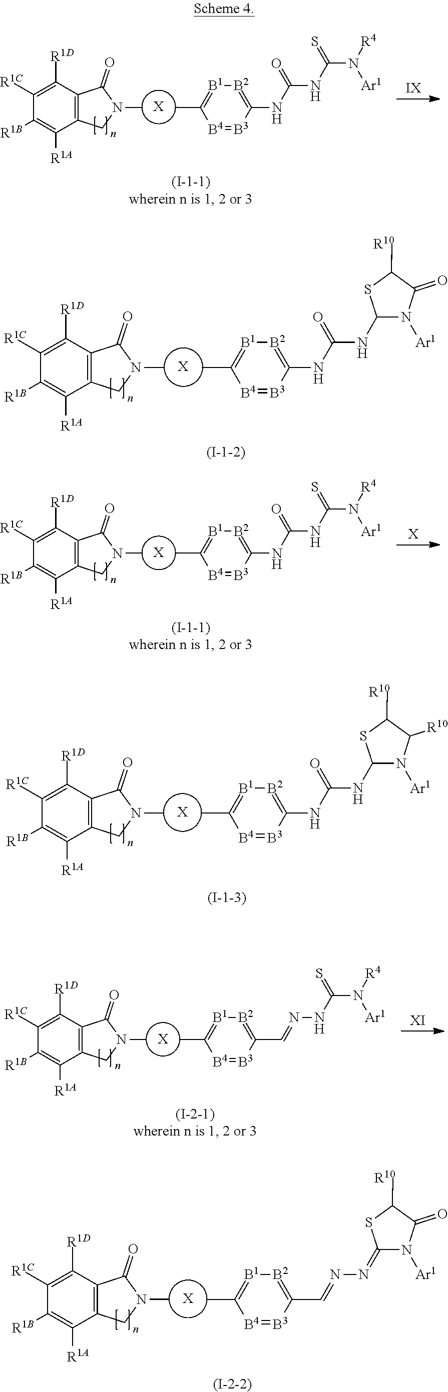

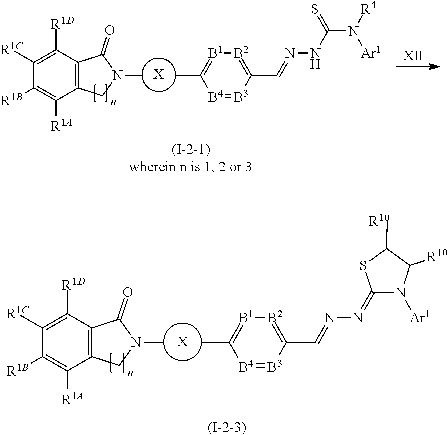

Compounds of formula (I-1-1) and (I-2-1) where R⁴ is H, can be converted into a variety of cyclized analogs of formulae (I-1-2) and (I-1-3) or (I-2-2) and (I-2-3), respectively. Cyclization can be achieved by treatment of compounds of formula (I-1-1) or (I-2-1) with α-halo esters such as methyl bromoacetate to form compounds of formula (I-1-2) or (I-2-2) unsubstituted or substituted with R¹⁰. Compounds of formula (I-1-3) or (I-2-3) unsubstituted or mono- or di-substituted with R¹⁰ can be prepared by treatment of compounds of formula (I-1-1) or (I-2-1) with vicinal dihalides. For steps (IX) and (XI), use of sodium acetate in aprotic solvent such as ACN, at temperatures ranging from about 20° C. to about 70° C. is preferred. For steps (X) and (XII), use of an inorganic base such as potassium carbonate in a solvent such as ACN or 2-butanone, at a temperature between about 0° C. and about 80° C., is preferred. All the above reactions can be performed analogously to the methods described in WO 2021/011722.

Compounds of formula (I-1-1) can be prepared analogously to the methods described in Scheme 1, Scheme 2 or Scheme 5. Compounds of formula (I-2-1) can be prepared analogously to the methods described in Scheme 6.

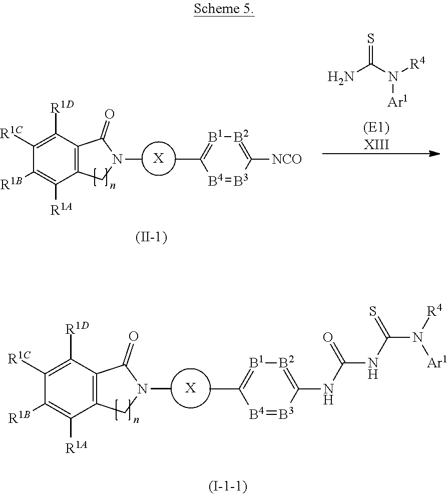

Compounds of the formula (I-1-1) can be prepared by treating aryl thiourea of formula (E1) with the isocyanate of formula (II-1) in the presence of inorganic bases such as cesium carbonate in an aprotic solvent. Compounds of formula (II-1) can be prepared analogously to the methods described in WO 2021/011722, WO 2014/204622 or by the methods described in Scheme 1. Compounds of formula (E1) can be prepared analogously to the methods described in J. Med. Chem. 2010, 53(10), 4198-4211 or in WO 2021/011722.

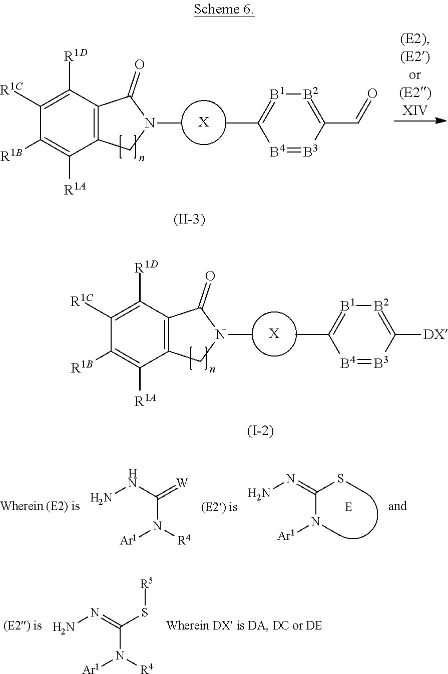

In one embodiment of Scheme 6, an aldehyde of the formula (II-3) is reacted with a compound of formula (E2), (E2′) or (E2″) in the presence or in the absence of a solvent. Suitable solvents are polar protic solvents. If the reaction is performed in the absence of a solvent, the compound of the formula (E2), (E2′) or (E2″) can also act as the solvent. Compounds of the formula (E2), (E2′) or (E2″) are commercially available or can be prepared using organic reactions analogy to method as described in March's Advanced Organic Chemistry 6th edition, Michael B. Smith and Jerry March, in Bioorg. Med. Chem., 2004, 12(17), 4633-4643 or in Bioorg. Med. Chem. Lett., 2005, 15(3), 539-543.

Compounds of formula (II-3) can be prepared analogously to the methods described in Scheme 7.

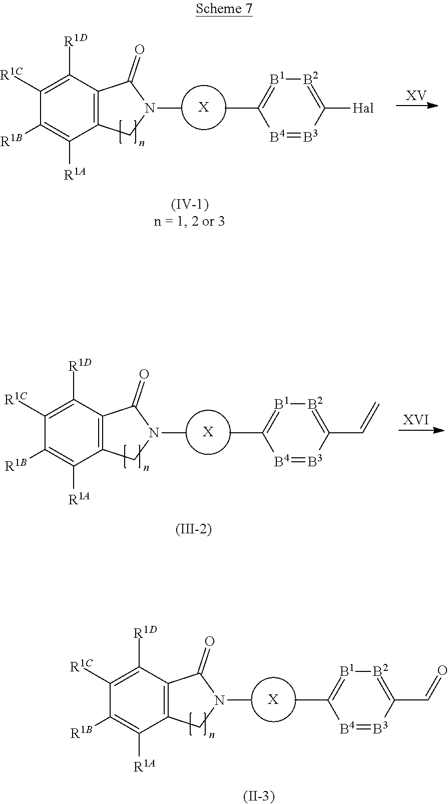

Reaction step (XV) can be performed by analogy to method described in WO 2015/051341. Reaction step (XVI) can be performed by analogy to method described in E. J. Med. Chem., 2012, 49, 310-323.

Compounds of the formula (IV-1) can be prepared by the methods described in Scheme 9.

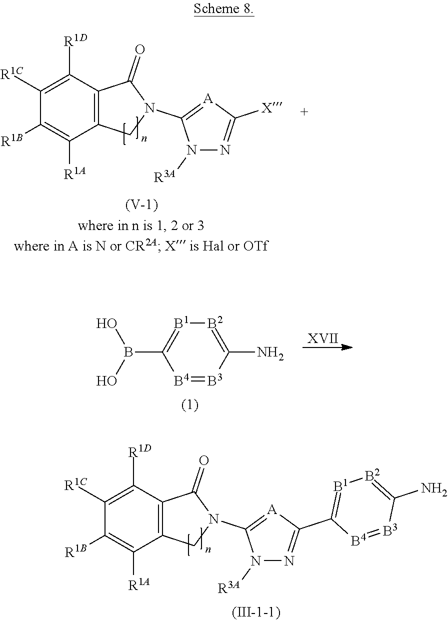

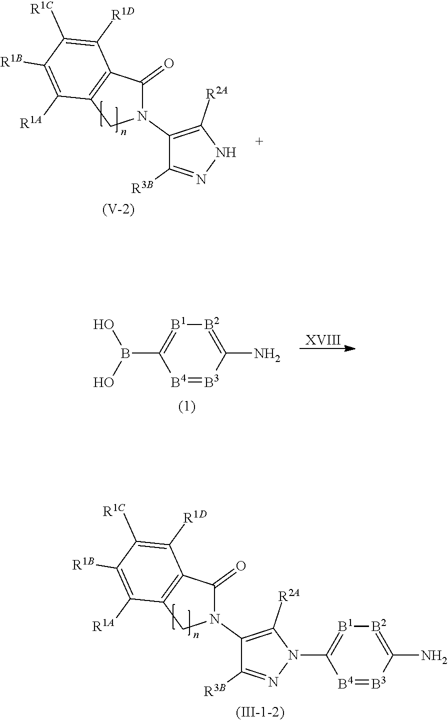

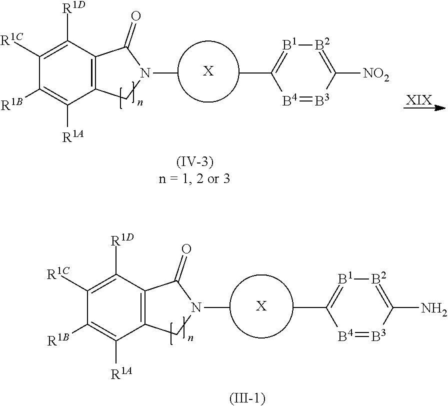

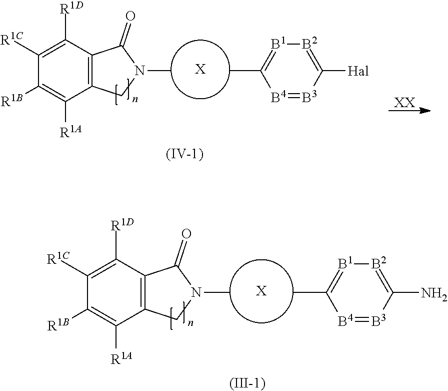

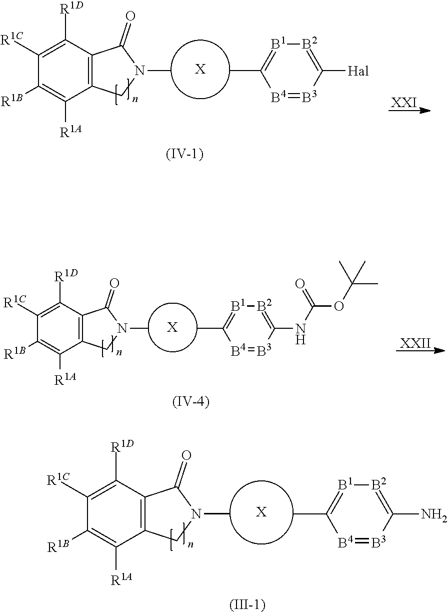

Compounds of formula (III-1-1) can be prepared with different synthetic routes. Step (XVII) can be performed via Suzuki cross coupling reaction starting from an appropriate aryl boronic acid/ester, such as precursor (1), as described in either Tetrahedron, 2009, 65(37), 7817-7824 or WO2018/075937.

Compounds of formula (III-1-2) can be prepared by Chan-Lam coupling with an appropriate boronic acid, such as precursor (1), as depicted in step (XVIII) and as described in Bioorg. Med. Chem., 17(13), 2009, 4708-4717. Compounds of the formula (III-1-2) can also be prepared by aromatic nucleophilic substitution of the appropriate 4-fluoroaniline, analogously to the method described for preparation of compounds of the formula (IV-2) in Scheme 9.

Compounds of formula (III-1) can also be prepared by reduction of nitro compounds of formula (IV-3) using reducing agents such as SnCl₂ in acid medium as shown in step (XIX).

Alternatively, compounds of formula (III-1) can also be prepared by reacting compounds of the formula (IV-1) with ammonia in the presence of a metal catalyst or its salts, preferably copper or its salts as depicted in step (XX) as described in Chem. Commun., 2009, 3035-3037.

Additionally, compounds of formula (III-1) can also be prepared in two steps from compounds of the formula (IV-1). Treatment of compounds of formula (IV-3) with tert-butyl carbamate in the presence of metal catalyst or its salts, preferably palladium or its salts to form compounds of formula (IV-4) in step (XXI), followed by Boc-deprotection using trifluoroacetic acid or diluted hydrochloric acid to form the desired compound in step (XXII). All these reactions are performed as described in March's Advanced Organic Chemistry 6th edition, Michael B. Smith and Jerry March.

Compounds of formula (V-1) and (V-2) can be prepared by methods described in Scheme 10 and Scheme 11. Compounds of formula (IV-3) can be prepared analogously to the methods described in Scheme 9. Compounds of formula (IV-1) can be prepared analogously to the methods described in Scheme 9.

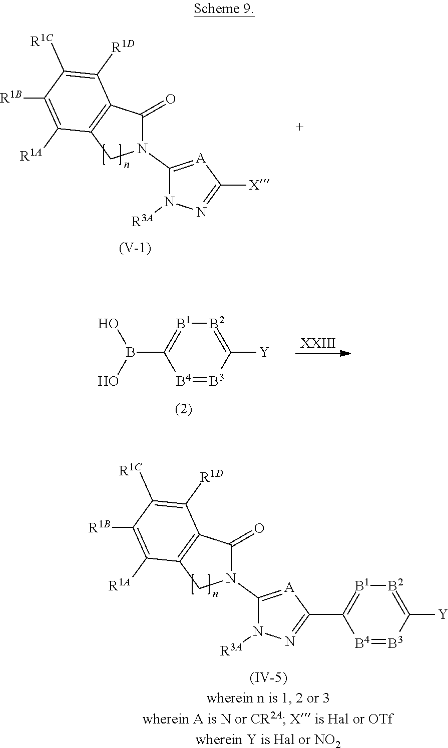

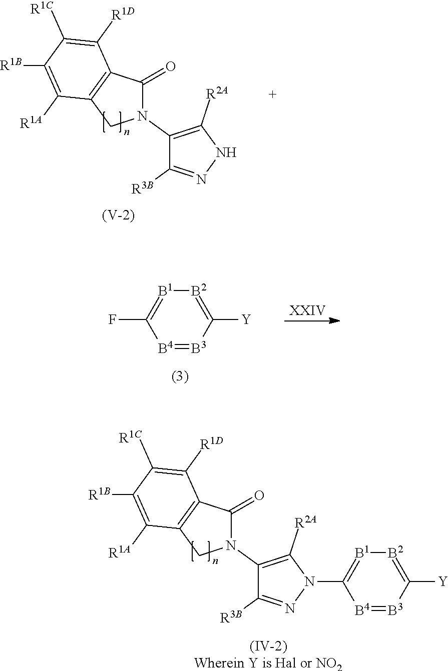

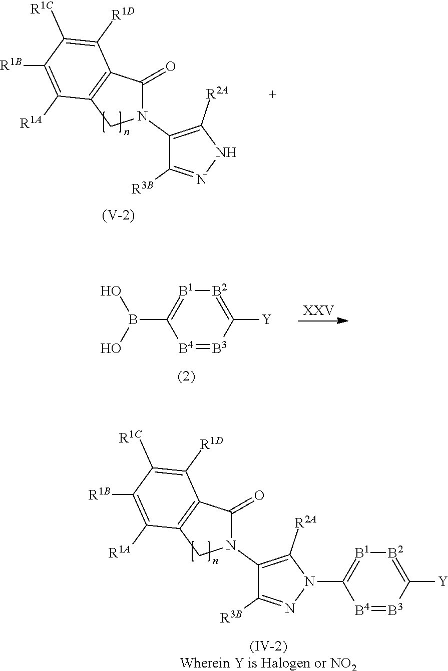

Step (XXIII) can be performed via Suzuki cross coupling reaction starting from an aryl boronic acid precursor (2) as described in Tetrahedron, 2009, 65(37), 7817-7824 or WO2018/075937. Similar reaction can also be carried with aryl boronate ester instead of aryl boronic acid precursor (2).

Step (XXIV) involves a nucleophilic aromatic substitution reaction between pyrazole of formula (IVa) and p-fluoro nitroarene or p-fluoro haloarene (3) as described in WO 2017/139274.

Step (XXV) can be performed via Chan-Lam coupling reaction starting from an aryl boronic acid such as precursor (2) as described in Chem. A Eur. J., 2017, 23(14), 3285-3290.

Compounds of formula (V-1) and (V-2) can be prepared by the methods described in Scheme 10 and Scheme 11.

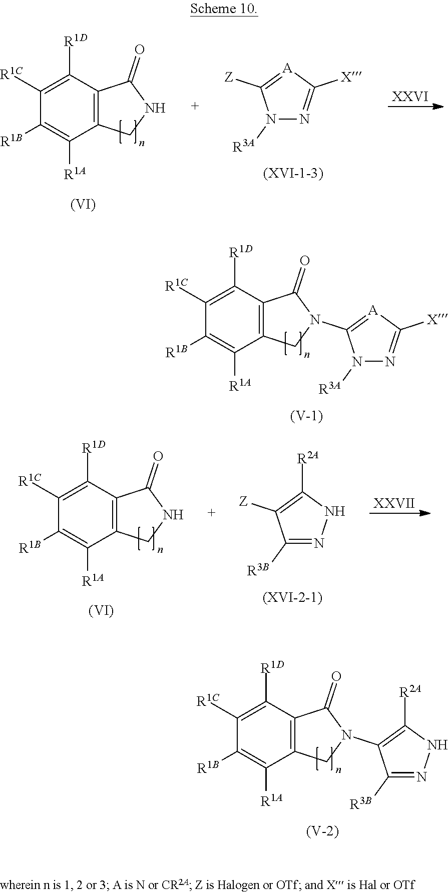

Compounds of the formula (V-1) can be prepared from the metal catalyzed Buchwald or Ullmann coupling of the lactam (VI) with the substituted heteroaryl ring (XVI-1-3) where Z is Hal or OTf, as in step (XXVI). Alternatively, compounds of the formula (V-1) can be prepared by nucleophilic aromatic substitution of the heteroaryl compounds (XVI-1-3), when Z is Hal, by (VI). Compounds of the formula (V-2) can be prepared from the metal catalyzed Buchwald or Ullmann coupling of the lactam (VI) with the substituted heteroaryl ring (XVI-2-1), as in step (XXVII). Alternatively, compounds of the formula (V-2) can be prepared by nucleophilic aromatic substitution of the heteroaryl compounds (XVI-2-1), when Z is Hal, by (VI).

Compounds of the formula (VI) can be purchased commercially or prepared according to the methods in Scheme 13.

Compounds of the formula (XVI-1-3) and (XVI-2-1) can be purchased commercially or prepared analogously according to the methods described in Scheme 14.

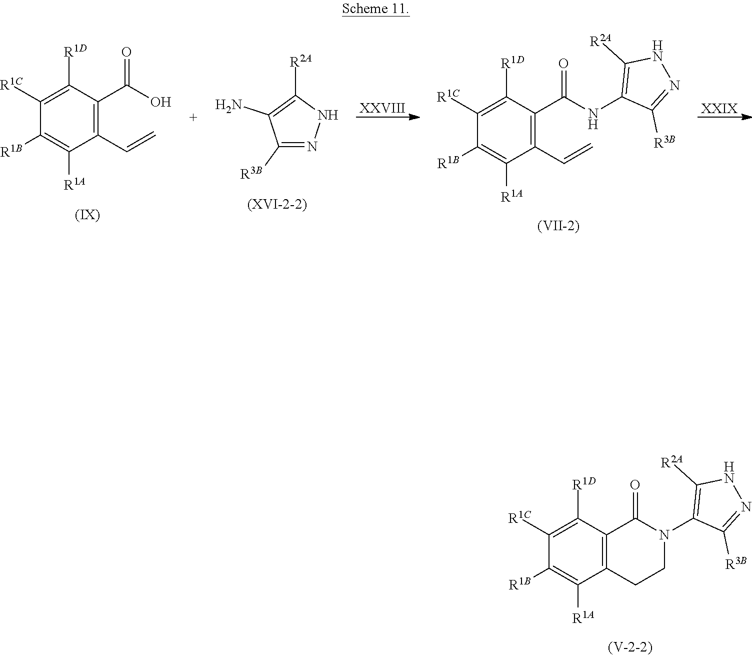

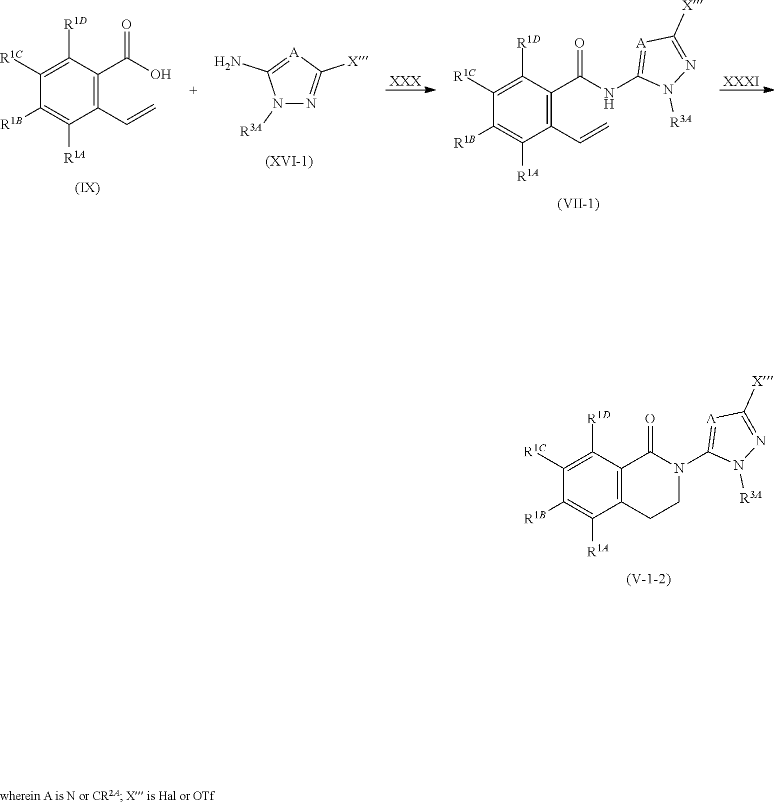

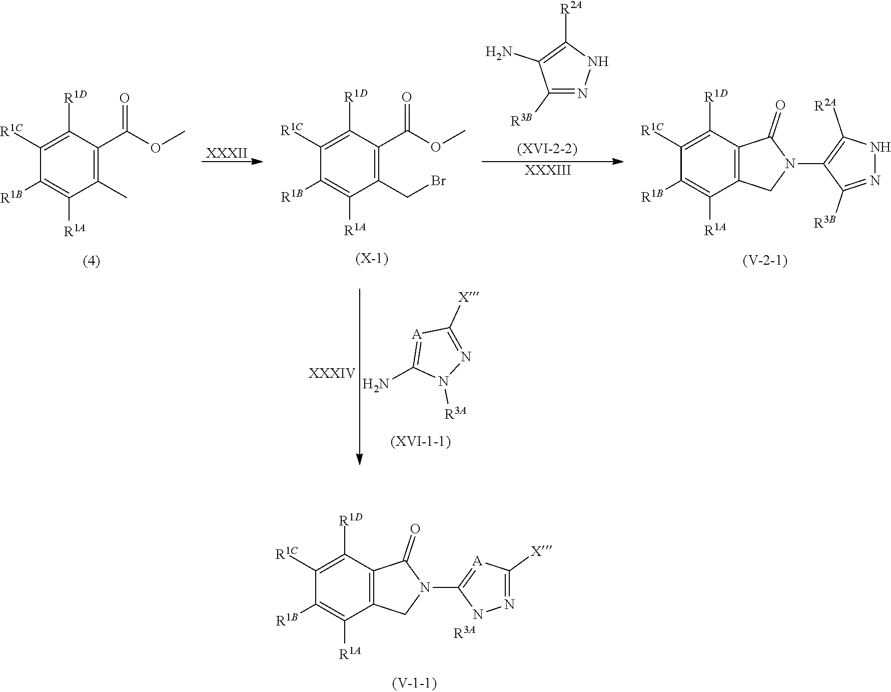

Compounds of the formula (V-2-2) can be prepared by the formation of the intermediates (VII-2) by amide coupling reagents, T3P, or by the in-situ generation of the acid chloride with thionyl chloride or oxalyl chloride and DMF, followed by the subsequent cyclization in the presence of an organic base such potassium tert-butoxide in DMF as the solvent.

Similarly, compounds of the formula (V-1-2) can be prepared by the formation of the intermediates (VII-1) by amide coupling reagents, T3P, or by the in-situ generation of the acid chloride with thionyl chloride or oxalyl chloride and DMF, followed by the subsequent cyclization in the presence of an organic base such potassium tert-butoxide in DMF as the solvent.

Compounds of the formula (V-2-1) and (V-1-1) can be prepared in 2 steps, first by bromination of the methyl group of starting materials of the formula (4) with N-bromosuccinimide in the presence of azobisisobutylnitrile as a catalyst to form the intermediates of the formula (X-1), as in step (XXXII), as described in the methods of Bioorganic & Medicinal Chemistry Letters, 2016, 26(10), 2526-2530. Compounds of the formula (V-1-1) and (V-2-1) can then be formed by heating with compounds of the formula (XVI-2-2) or (XVI-1-1), under basic conditions, as in steps (XXXIII) and (XXXIV), respectively, as described in WO2006/113140.

Compounds of the formula (IX) can be prepared by the methods described in Scheme 12.

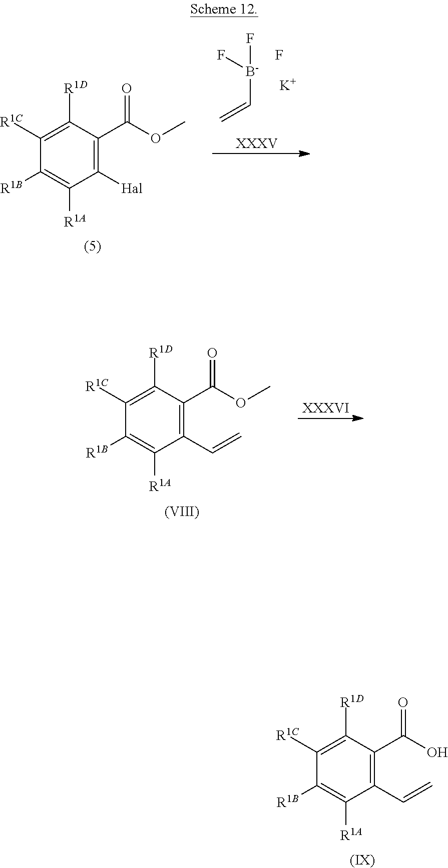

Compounds of the formula (VIII) can be prepared the corresponding building blocks (5) by palladium catalyzed Suzuki coupling, employing potassium vinyltrifluoroborate as in step (XXXV), or by the methods described in Org. Chem. Front., 2022, 9(4), 989-994. Compounds of the formula (IX) can then be prepared by hydrolysis of compounds (VIII) employing an inorganic base, for example NaOH or LiOH, in a mixture of water and a water-miscible organic solvent, for example THF or MeOH, or as described in March's Advanced Organic Chemistry 6^{th} edition, Michael B. Smith and Jerry March.

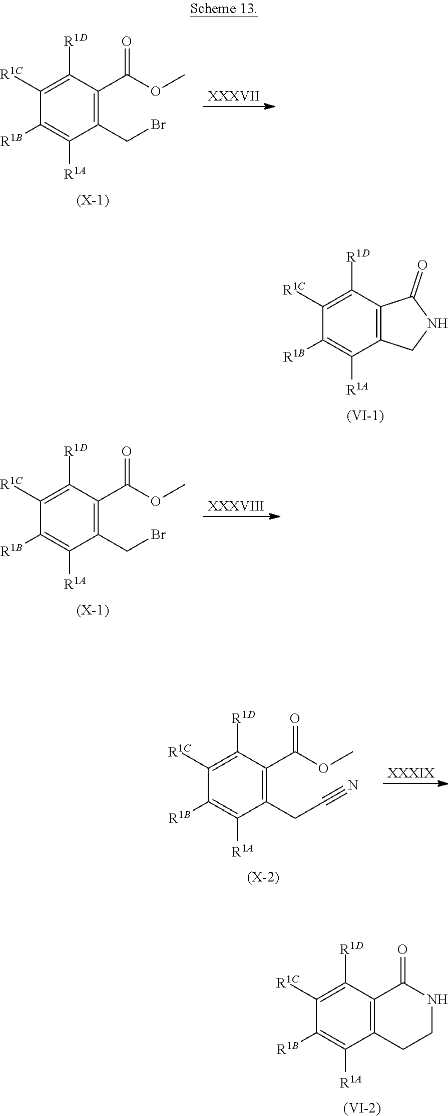

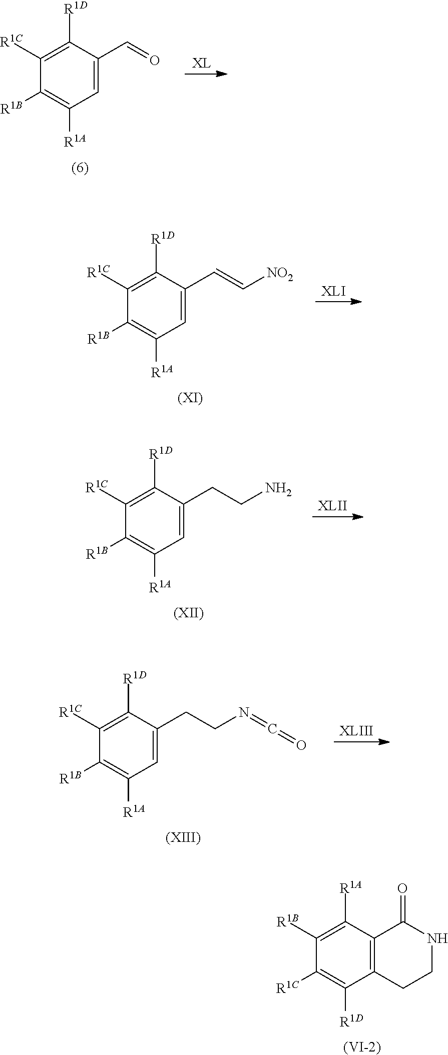

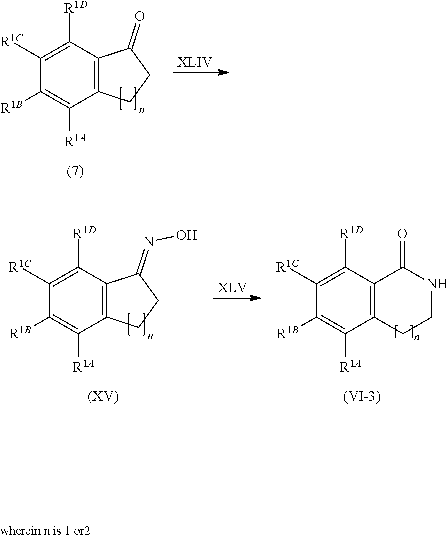

Compounds of the formula (VI-1) can be prepared from compounds (X-1) by heating with aqueous ammonia, as in step (XXXVII), or as described in Bioorg. Med. Chem. Lett., 2012, 22(2), 814-819.

Compounds of the formula (VI-2) can be prepared from compounds of the formula (X-1) in two steps, by first reacting with sodium cyanide as in step (XXXVIII) to give compounds of the formula (X-2), followed by cyclisation under reductive conditions, such as with hydrogen gas over Raney nickel, or with platinum oxide and a mild organic acid such as acetic acid, as in step (XXXIX).

Compounds of the formula (VI-2) can also be prepared from the corresponding aldehydes (6) in 5 steps. Firstly, Henry condensation with nitromethane as in step (XL) is performed, followed by reduction with LiAlH or DIBAL to the corresponding amines (XII). Isocyanate intermediates (XIII) can be prepared by treating compounds of the formula (XII) with phosgene, diphosgene, triphosgene or carbonyldiimidazole in a mixed solvent system and in the presence of a base as described in March's Advanced Organic Chemistry 6th edition, Michael B. Smith and Jerry March. Finally, compounds of the formula (VI-2) can then be prepared from the corresponding isocyanates (XIII) by treating with trifluoromethanesulfonic acid at 0° C., as described in the methods of J. Org. Chem. 2012, 77(20), 9313-9328.

Compounds of the formula (VI-2) can be prepared in 2 steps from the building blocks (7) by first condensing with hydroxylamine to give compounds of the formula (XV), as in step (XLIV), as described in Tet. Lett. 2017, 58(23), 2240-2243, followed by Beckmann rearrangement, as in the methods described in Bioorg. Med. Chem. Lett. 2002, 12(3), 387-390.

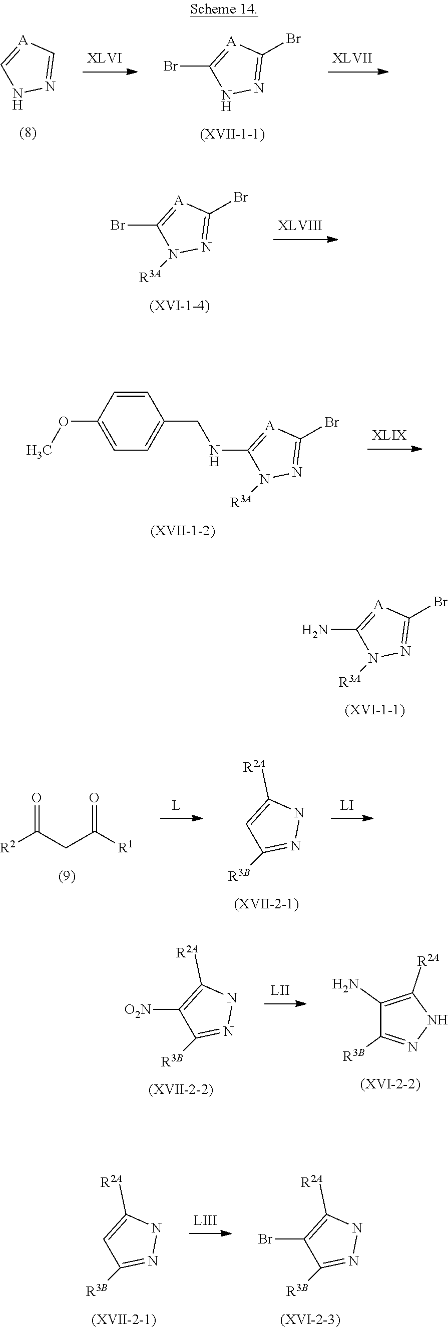

Compounds of formula (XVI-1-4) and (XVI-1-1) can be prepared from commercially available building blocks of the formula (8). Step (XLVI) includes bromination by reacting the compounds of formula (8) with bromine in presence of a weak base like sodium acetate, a protic solvent like ethanol and water. Step (XLVII) includes alkylation by reacting with the corresponding commercially available alkyl halides preferably iodides or bromides in presence of bases like cesium carbonate and polar aprotic solvent like DMF, analogous to as described in March's Advanced Organic Chemistry 6^{th} edition, Michael B. Smith and Jerry March. Step (XLVIII) includes introduction of protecting group such as 2,4-dimethoxybenzylamine. Step (XLIX) involves deprotection to give of compounds of formula (XVII-1-1) by reacting compound of formula (XVII-1-2) with 5N Hydrochloric acid. All these steps can be performed as described in March's Advanced Organic Chemistry 6th edition, Michael B. Smith and Jerry March.

Compounds of formula (XVI-2-1) can be prepared from a commercially available 1,3 diketone derivative (9) by reacting it with hydrazine, analogously to the method described in WO 2016/044666. Then nitration of compounds of formula (XVII-2-1) using a mixture of nitric acid and sulfuric acid in an aprotic solvent such as dichloroethane can produce compounds of formula (XVII-2-2). Compounds of formula (XVII-2-2) can undergo reduction to compounds of formula (XVI-2-2) using reducing agents such as SnCl₂ in acid medium or Fe with NH₄Cl in a mixture of Ethanol and water as shown in step (LII), or as described in March's Advanced Organic Chemistry 6th edition, Michael B. Smith and Jerry March.

Compounds of the formula (XVI-2-3) can be prepared by treating compounds (XVII-2-1) with N-bromosuccinimide, as described in the methods of ACS Med. Chem. Lett., 2019, 10 (4), 627-632

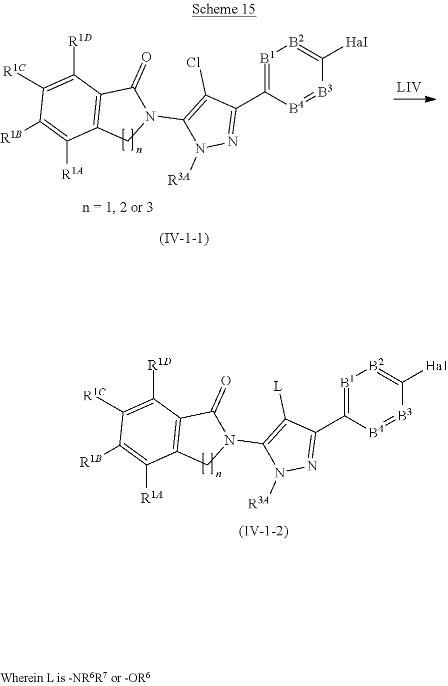

Compounds of formula (IV-1-2) can be prepared from compounds of formula (IV-1-1) in one step. Nucleophilic aromatic substitution of the chloro group of compounds of formula (IV-1-1) can be performed by reacting with the corresponding commercially available substituted amines, in presence of bases like TEA, and polar aprotic solvents like DMF to get compounds of formula (IV-1-2). Compounds of the formula (IV-1-2) wherein L is NR⁶R⁷ can be prepared by amination of compounds of the formula (IV-1-1) in presence of bases like TEA and polar aprotic solvents such as DMF. Compounds of the formula (IV-1-2) wherein L is —OR⁶ can be prepared analogously according to the methods described in WO2021/204626 or in US2016/0185785.

Compounds of the formula (IV-1-1) can be prepared according to the methods described in scheme 16.

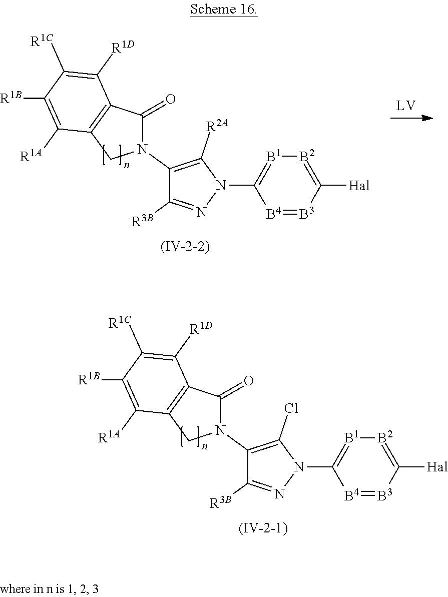

Compounds of formula (IV-2-1) can be prepared by treating compounds of formula (IV-2-2) with electrophilic halogenating agent such as Palau'Chlor in a non-polar aprotic solvent like chloroform analogous to as described in March's Advanced Organic Chemistry 6^{th} edition, Michael B. Smith and Jerry March.

Individual compounds of formula I can also be prepared by derivatisation of other compounds of formula I or the intermediates thereof.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

Unless otherwise indicated, the term “compound(s) according to the invention” or “compound(s) of the invention” or “compound(s) of formula (I)”, refers to the compounds of formula I.

The term “compound(s) according to the invention”, or “compounds of formula I” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The term “composition(s) according to the invention” or “composition(s) of the present invention” encompasses composition(s) comprising at least one compound of formula I according to the invention as defined above. The compositions of the invention are preferably agricultural or veterinary compositions.

Depending on the substitution pattern, the compounds according to the invention may have one or more centers of chirality, in which case they are present as mixtures of enantiomers or diastereomers. The invention provides both the single pure enantiomers or pure diastereomers of the compounds according to the invention, and their mixtures and the use according to the invention of the pure enantiomers or pure diastereomers of the compounds according to the invention or their mixtures. Suitable compounds according to the invention also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula I, i.e. to single enantiomers or diastereomers, as well as to mixtures thereof.

The compounds according to the invention may be amorphous or may exist in one or more different crystalline states (polymorphs) which may have different macroscopic properties such as stability or show different biological properties such as activities. The present invention relates to amorphous and crystalline compounds according to the invention, mixtures of different crystalline states of the respective compounds according to the invention, as well as amorphous or crystalline salts thereof.

The term “tautomers” encompasses isomers, which are derived from the compounds of formula I by the shift of an H-atom involving at least one H-atom located at a nitrogen, oxygen or sulphur atom. Examples of tautomeric forms are keto-enol forms, imine-enamine forms, urea-isourea forms, thiourea-isothiourea forms, (thio)amide-(thio) imidate forms etc.

The term “stereoisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term N-oxides relates to a form of compounds I in which at least one nitrogen atom is present in oxidized form (as NO). To be more precise, it relates to any compound of the present invention which has at least one tertiary nitrogen atom that is oxidized to an N-oxide moiety. N-oxides of compounds I can in particular be prepared by oxidizing e.g. the ring nitrogen atom of an N-heterocycle, e.g. a pyridine or pyrimidine ring present in Ar or R¹¹, or an imino-nitrogen present in central tricyclic core, with a suitable oxidizing agent, such as peroxo carboxylic acids or other peroxides. The person skilled in the art knows if and in which positions compounds of the present invention may form N-oxides.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally or veterinarily acceptable salts are especially the salts of those cations or the acid addition salts of those acids whose cations and anions, which are known and accepted in the art for the formation of salts for agricultural or veterinary use respectively, and do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or —CH₂-phenyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium. Suitable acid addition veterinarily acceptable salts, e.g. formed by compounds of formula I containing a basic nitrogen atom, e.g. an amino group, include salts with inorganic acids, for example hydrochlorides, sulphates, phosphates, and nitrates and salts of organic acids for example acetic acid, maleic acid, dimaleic acid, fumaric acid, difumaric acid, methane sulfenic acid, methane sulfonic acid, and succinic acid.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. The plant propagation materials may be treated prophylactically with a plant protection compound either at or before planting or transplanting. Said young plants may also be protected before transplantation by a total or partial treatment by immersion or pouring.

The term “plants” comprises any types of plants including “modified plants” and in particular “cultivated plants”.

The term “modified plants” refers to any wild type species or related species or related genera of a cultivated plant.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp, or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-COA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coelop-tera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the CryIAb toxin), YieldGard® Plus (corn cultivars producing CryIAb and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the CryIAc toxin), Bollgard® | (cotton cultivars producing the CryIAc toxin), Bollgard® II (cotton cultivars producing CryIAc and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the CryIAb toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the CryIAc toxin) and 1507 from Pioneer Overseas Corporation, Belgium (corn cultivars producing the CryIF toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The organic moieties mentioned in the above definitions of the variables are—like the term halogen—collective terms for individual listings of the individual members. The prefix Cₙ-Cₘ indicates in each case the possible number of carbon atoms in the group.

The term “halogen” denotes in each case F, Br, Cl or I, in particular F, Cl or Br.

The term “alkyl” as used herein and in the alkyl moieties of alkoxy, alkylthio, and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”) or 1 to 6 (“C₁-C₆-alkyl”) carbon atoms. C₁-C₂-alkyl is CH₃ or C₂H₅. C₁-C₃-alkyl is additionally propyl and isopropyl, C₁-C₄-alkyl is additionally butyl, 1-methylpropyl (sec-butyl), 2-methylpropyl (isobutyl) or 1,1-dimethylethyl (tert-butyl). C₁-C₆-alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”) or 1 to 6 (“C₁-C₆-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like.

The term “alkylene” (or alkanediyl) as used herein in each case denotes an alkyl radical as defined above, wherein one hydrogen atom at any position of the carbon backbone is replaced by one further binding site, thus forming a bivalent moiety. Alkylene has preferably 1 to 6 carbon atoms (C₁-C₆-alkylene), 2 to 6 carbon atoms (C₂-C₆-alkylene), in particular 1 to 4 carbon atoms (C₁-C₄-alkylene) or 2 to 4 carbon atoms (C₂-C₄-alkylene). Examples of alkylene are methylene (CH₂), 1,1-ethanediyl, 1,2-ethanediyl, 1,3-propandiyl, 1,2-propandiyl, 2,2-propandiyl, 1,4-butanediyl, 1,2-butanediyl, 1,3-butanediyl, 2,3-butanediyl, 2,2-butanediyl, 1,5-pentanediyl, 2,2-dimethylpropan-1,3-diyl, 1,3-dimethyl-1,3-propandiyl, 1,6-hexanediyl etc.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”) or 2 to 6 (“C₂-C₆-alkenyl) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like.

The term “alkynyl” as used herein refers to straight-chain or branched hydrocarbon groups having 2 to 3 (“C₂-C₃-alkynyl”), 2 to 4 (“C₂-C₄-alkynyl”) or 2 to 6 (“C₂-C₆-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “cycloalkyl” as used herein refers to mono- or bi- or polycyclic saturated hydrocarbon radicals having in particular 3 to 6 (“C₃-C₆-cycloalkyl”) or 3 to 5 (“C₃-C₅-cycloalkyl”) or 3 to 4 (“C₃-C₄-cycloalkyl”) carbon atoms. Examples of monocyclic radicals having 3 to 4 carbon atoms comprise cyclopropyl and cyclobutyl. Examples of monocyclic radicals having 3 to 5 carbon atoms comprise cyclopropyl, cyclobutyl and cyclopentyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “cycloalkoxy” as used herein refers to a cycloalkyl radical, in particular a monocyclic cycloalkyl radical, as defined above having in particular 3 to 6 (“C₃-C₆-cycloalkoxy”) or 3 to 5 (“C₃-C₅-cycloalkoxy”) or 3 to 4 (“C₃-C₄-cycloalkoxy”) carbon atoms, which is bound via an oxygen atom to the remainder of the molecule.

The term “cycloalkyl-C₁-C₄-alkyl” refers to a C₃-C₈-cycloalkyl (“C₃-C₈-cycloalkyl-C₁-C₄-alkyl”), preferably a C₃-C₆-cycloalkyl (“C₃-C₆-cycloalkyl-C₁-C₄-alkyl”), more preferably a C₃-C₄-cycloalkyl (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C₁-C₄-alkyl, are cyclopentylmethyl, cyclopentylethyl, cyclopentylpropyl, cyclohexylmethyl, cyclohexylethyl and cyclohexylpropyl.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is OCH₃ or OCH₅. C₁-C₃-Alkoxy is additionally, for example, n-propoxy and 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₆-alkoxy-C₁-C₄-alkyl” as used herein, refers to a straight-chain or branched alkyl having 1 to 4 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “alkoxyalkoxy” as used herein refers to an alkoxyalkyl radical, in particular a C₁-C₆-alkoxy-C₁-C₄-alkyl radical, as defined above, which is bound via an oxygen atom to the remainder of the molecule. Examples thereof are OCH₂—OCH₃, OCH₂—OC₂H₅, n-propoxymethoxy, OCH₂—OCH(CH₃)₂, n-butoxymethoxy, (1-methylpropoxy)methoxy, (2-methylpropoxy)methoxy, OCH₂—OC(CH₃)₃, 2-(methoxy)ethoxy, 2-(ethoxy)ethoxy, 2-(n-propoxy)ethoxy, 2-(1-methylethoxy)ethoxy, 2-(n-butoxy)ethoxy, 2-(1-methylpropoxy)ethoxy, 2-(2-methylpropoxy)ethoxy, 2-(1,1-dimethylethoxy)ethoxy, etc.

The substituent “oxo” replaces a CH₂ by a C(═O) group.

The term “aryl” relates to phenyl and bi- or polycyclic carbocycles having at least one fused phenylene ring, which is bound to the remainder of the molecule. Examples of bi- or polycyclic carbocycles having at least one phenylene ring include naphthyl, tetrahydronaphthyl, indanyl, indenyl, anthracenyl, fluorenyl etc.

The term “5- to 6-membered carbocyclic group” as used herein refers to cyclopentane and cyclohexane rings which may contain heteroatoms selected from N, O, and S.

The term “aryl-C₁-C₄-alkyl” relates to C₁-C₄-alkyl, as defined above, wherein one hydrogen atom has been replaced by an aryl radical, in particular a phenyl radical. Particular examples of aryl-C₁-C₄-alkyl include-CH₂-phenyl, 1-phenethyl, 2-phenetyl, 1-phenylpropyl, 2-phenylpropyl, 3-phenyl-1-propyl and 2-phenyl-2-propyl.

The term “aryloxy-C₁-C₄-alkyl” relates to C₁-C₄-alkyl, as defined above, wherein one hydrogen atom has been replaced by an aryloxy radical, in particular a phenoxy radical. Particular examples of aryloxy-C₁-C₄-alkyl include phenoxymethyl, 1-phenoxyethyl, 2-phenoxyetyl, 1-phenoxypropyl, 2-phenoxypropyl, 3-phenoxy-1-propyl and 2-phenoxy-2-propyl.

The term “aryl-C₁-C₄-carbonyl” relates to aryl as defined above, in particular a phenyl radical, which is bound by a carbonyl to the remainder of the molecule. Particular examples of arylcarbonyl include benzoyl, 1-naphthoyl and 2-naphthoyl.

The term “hetaryl” relates to aromatic heterocyclyl or heterocycles having either 5 or 6 ring atoms (5- or 6-membered hetaryl) and being monocyclic or 8, 9 or 10 ring atoms and being bicyclic. Hetaryl will generally have at least one ring atom selected from O, S and N, which in case of N may be an imino-nitrogen or an amino-nitrogen, which carries hydrogen or a radical different from hydrogen. Hetaryl may have 1, 2, 3 or 4 further nitrogen atoms as ring members, which are imino nitrogens. Examples of 5- or 6-membered hetaryl include 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 1,3,4-oxadiazolyl-2-yl, 1,3,4-thiadiazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl and 1,3,5-triazin-2-yl. Examples of 8-, 9- or 10-membered hetaryl include, for example, quinolinyl, isoquinolinyl, cinnolinyl, indolyl, indolizinyl, isoindolyl, indazolyl, benzofuryl, benzothienyl, benzo[b]thiazolyl, benzoxazolyl, benzthiazolyl, benzimidazolyl, imidazo[1,2-a]peridine-2-yl, thieno[3,2-b]pyridine-5-yl, imidazo-[2,1-b]-thiazol-6-yl and 1,2,4-triazolo[1,5-a]peridine-2-yl. Examples of N-bound 5-, 6-, 7 or 8-membered saturated heterocyclyl or heterocycles include: pyrrolidin-1-yl, pyrazolidin-1-yl, imidazolidin-1-yl, oxazolidin-3-yl, isoxazolidin-2-yl, thiazolidin-3-yl, isothiazolidin-2-yl, piperidin-1-yl, piperazin-1-yl, morpholin-4-yl, thiomorpholin-4-yl, 1-oxothiomorpholin-4-yl, 1,1-dioxothiomorpholin-4-yl, azepan-1-yl and the like.

The term “hetaryl-C₁-C₄-alkyl” relates to C₁-C₄-alkyl, as defined above, wherein one hydrogen atom has been replaced by a hetaryl radical, in particular a pyridyl radical. Particular examples of hetaryl-C₁-C₄-alkyl include 2-pyridylmethyl, 3-pyridylmethyl, 4-pyridylmethyl, 1-(2-pyridyl)ethyl, 2-(2-pyridyl)ethyl, 1-(3-pyridyl)ethyl, 2-(3-pyridyl)ethyl, 1-(4-pyridyl)ethyl, 2-(4-pyridyl)ethyl etc.

The term “hetaryloxy-C₁-C₄-alkyl” relates to C₁-C₄-alkyl, as defined above, wherein one hydrogen atom has been replaced by an hetaryloxy radical, in particular a pyridyloxy radical. Particular examples of hetaryloxy-C₁-C₄-alkyl include 2-pyridyloxymethyl, 3-pyridyloxymethyl, 4-pyridyloxymethyl, 1-(2-pyridyloxy)ethyl, 2-(2-pyridyloxy)ethyl, 1-(3-pyridyloxy)ethyl, 2-(3-pyridyloxy)ethyl, 1-(4-pyridyloxy)ethyl, 2-(4-pyridyloxy)ethyl etc.

The term “hetaryl-C₁-C₄-carbonyl” relates to hetaryl as defined above, in particular a C-bound hetaryl radical, e.g. 2-, 3- or 4-pyridyl, 2- or 3-thienyl, 2- or 3-furyl, 1-, 2- or 3-pyrrolyl, 2- or 4-pyrimidinyl, pyridazinyl, 1-, 3- or 4-pyrazolyl, 1-, 2- or 4-imidazolyl radical, which is bound by a carbonyl to the remainder of the molecule.

The term “substituted” if not specified otherwise refers to substituted with 1, 2, or up to maximum possible number of substituents. If substituents as defined in compounds of formula I are more than one then they are independently from each other are same or different if not mentioned otherwise.

With respect to the variables, the embodiments of the compounds of the formula I are:

In one preferred embodiment, maximum two of B¹, B,² and B³ are N.

In another preferred embodiment, B¹ is CR^{B1}, B² is CR^{B2}, and B³ is CR^{B3}.

In another preferred embodiment, B¹ is N, B² is CR^{B2}, and B³ is CR^{B3}.

In another preferred embodiment, B¹ is CR^{B1}, B² is N, and B³ is CR^{B3}.

In another preferred embodiment, B¹ is CR^{B1}, B² is N, and B³ is N.

In another preferred embodiment, B¹ is N, B² is N, and B³ is CR^{B3}.

In another preferred embodiment, B¹ is CR^{B1}, and B² is N or CR^{B2}, B³ is N or CR^{B3}.

In another preferred embodiment, B³ is CR^{B3}, and B¹ is N or CR^{B2}, B² is N or CR^{B3}.

In various particularly preferred embodiments B¹ is CR^{B1}, B² is CR^{B2}, and B³ is CR^{B3} or B¹ is N, B² is CR^{B2}, and B³ is CR^{B3}.

In one preferred embodiment, R^{B1}, R^{B2}, R^{B3}, and RB⁴ independently of each other are selected from H, halogen, CN, C₁-C₆-alkyl, and C₁-C₆-alkoxy, wherein the alkyl, alkoxy moieties are unsubstituted or substituted with halogen, or CN.

In another preferred embodiment, R^{B1}, R^{B2}, R^{B3}, and RB⁴ independently of each other are selected from H, halogen, CN, and C₁-C₆-alkyl, wherein the alkyl moieties are unsubstituted or substituted with halogen, or CN.

In another preferred embodiment, R^{B1}, R^{B2}, R^{B3}, and RB⁴ independently of each other are selected from H, halogen, CN, and C₁-C₆-alkyl, wherein the alkyl moieties are unsubstituted or substituted with halogen.
In another preferred embodiment, R^{B1}, R^{B2}, R^{B3}, and RB⁴ independently of each other are selected from H, halogen, CN, and C₁-C₆-alkyl, wherein the alkyl moieties are unsubstituted;
In another preferred embodiment, R^{B1}, R^{B2}, R^{B3}, and RB⁴ independently of each other are selected from H, halogen, and C₁-C₆-alkyl, wherein the alkyl moieties are unsubstituted;
In another preferred embodiment, R^{B1}, R^{B2}, R^{B3}, and RB⁴ independently of each other are selected from H and halogen;
In another preferred embodiment, R^{B1}, R^{B2}, R^{B3}, and RB⁴ independently of each other are selected from H, halogen, and CN;
In another preferred embodiment, R^{B1}, R^{B2}, R^{B3}, and RB⁴ independently of each other are selected from H, Cl, F, Br, and CH₃;
In another preferred embodiment, R^{B2}, RB³ and RB⁴ are H, and RB¹ is F or Cl;
In another preferred embodiment, R^{B1}, RB³ and RB⁴ are H, and RB² is F, Cl, Br or CH₃;
In one preferred embodiment, R^{1A}, R^{1B}, R^{1C} and R^{1D} independently of each other are selected from H, halogen, CN, C₁-C₆-alkyl, and C₁-C₆-alkoxy, wherein the alkyl, alkoxy moieties are unsubstituted or substituted with halogen or CN;
In another preferred embodiment, R^{1A}, R^{1B}, R^{1C} and R^{1D} independently of each other are selected from H, halogen, CN, C₁-C₆-alkyl, and C₁-C₆-alkoxy, wherein the alkyl, alkoxy moieties are unsubstituted or substituted with halogen;
In another preferred embodiment, R^{1A}, R^{1B}, R^{1C} and R^{1D} independently of each other are selected from H, halogen, CN, and C₁-C₆-alkoxy, wherein the alkoxy moieties are unsubstituted or substituted with halogen or CN;
In another preferred embodiment, R^{1A}, R^{1B}, R^{1C} and R^{1D} independently of each other are selected from H, halogen, CN, C₁-C₆-haloalkyl, and C₁-C₆-haloalkoxy;
In another preferred embodiment, R^{1A}, R^{1B}, R^{1C} and R^{1D} independently of each other are selected from H and C₁-C₆-haloalkoxy;
In another preferred embodiment, R^{1A}, R^{1B}, R^{1C} and R^{1D} independently of each other are selected from H, Cl, F, Br, CN, CF₃, and OCF₃;
In another preferred embodiment, R^{1A}, R^{1B}, R^{1C} and R^{1D} independently of each other are selected from H, Cl, F, CN, CF₃, and OCF₃;
In another preferred embodiment, R^{1A}, R^{1B}, R^{1C} and R^{1D} independently of each other are selected from H and OCF₃;
In another preferred embodiment, R^{1A} and R^{1C} are H, and R^{1B} and R^{1D} are independently of each other are selected from H, F, Cl, CN, OCF₃, and CF₃;
In another preferred embodiment, R^{1A}, R^{1C}, and R^{1D} are H, and R^{1B} is selected from F, Cl, CN, OCF₃, and CF₃;
In another preferred embodiment, R^{1A} R^{1B}, and R¹⁰ are H, and R^{1D} is F; In another preferred embodiment, R^{1B} is other than H;
In another preferred embodiment, R^{1C} is other than H;
In another preferred embodiment, R^{1D} is other than H;
In one preferred embodiment, n is 1 or 2;
In another preferred embodiment, n is 2;
In another preferred embodiment, n is 1;
In one preferred embodiment, X is X1;
In another preferred embodiment, X is X2;
In one preferred embodiment, A is N;

In another preferred embodiment, A is CR^{2A}.
In one preferred embodiment, R^{2A} is H, C₁-C₆-alkyl, C₃-C₆-cycloalkyl, or halogen, wherein the alkyl and cycloalkyl moieties are unsubstituted or substituted with halogen or CN;
In another preferred embodiment, R^{2A} is H, halogen, or C₁-C₆-alkyl;
In another preferred embodiment, R^{2A} is H or C₁-C₆-alkyl;
In another preferred embodiment, R^{2A} is H, or C₁-C₆-alkyl, wherein the alkyl moieties are unsubstituted or substituted with halogen or CN;
In another preferred embodiment, R^{2A} is C₁-C₆-alkyl, which is unsubstituted;
In another preferred embodiment, R^{2A} is C₁-C₆-alkyl, which is substituted with halogen or CN;
In another preferred embodiment, R^{2A} is C₃-C₆-cycloalkyl, which is unsubstituted or substituted with halogen or CN;
In another preferred embodiment, R^{2A} is C₃-C₆-cycloalkyl, which is unsubstituted;
In another preferred embodiment, R^{2A} is C₃-C₆-cycloalkyl, which is substituted with halogen or CN;
In another preferred embodiment, R^{2A} is H or halogen;
In another preferred embodiment, R^{2A} is H;
In another preferred embodiment, R^{2A} is halogen;
In another preferred embodiment, R^{2A} is H, halogen, or CH₃;
In another preferred embodiment, R^{2A} is H, Cl, or CH₃;
In another preferred embodiment, R^{2A} is H or CH₃;
In another preferred embodiment, A is N, C(C₁-C₆-alkyl), or CH;
In another preferred embodiment, A is N, C(CH₃), or CH;
In one preferred embodiment, R^{3A} is H, C₁-C₆-alkyl, or C₃-C₆-cycloalkyl, wherein the alkyl and cycloalkyl moieties are unsubstituted or substituted with halogen, CN or C₁-C₆-alkoxy;
In another preferred embodiment, R^{3A} is H or C₁-C₆-alkyl;
In another preferred embodiment, R^{3A} is H, or C₁-C₆-alkyl, wherein the alkyl moieties are unsubstituted or substituted with halogen or C₁-C₆-alkoxy;
In another preferred embodiment, R^{3A} is C₁-C₆-alkyl, which is unsubstituted;
In another preferred embodiment, R^{3A} is C₁-C₆-alkyl, which is substituted with halogen, CN or C₁-C₆-alkoxy;
In another preferred embodiment, R^{3A} is C₃-C₆-cycloalkyl, which is unsubstituted or substituted with halogen or C₁-C₆-alkoxy;
In another preferred embodiment, R^{3A} is C₃-C₆-cycloalkyl, which is unsubstituted; for example cyclopropyl;
In another preferred embodiment, R^{3A} is C₃-C₆-cycloalkyl, which is substituted with halogen or C₁-C₆-alkoxy;
In another preferred embodiment, R^{3A} is CH₃ or C₂H₅;
In another preferred embodiment, R^{3A} is H;
In another preferred embodiment, R^{3A} is H, CH₃ or C₂H₅;
In one preferred embodiment, R^{3B} is H or C₁-C₆-alkyl, wherein the alkyl and cycloalkyl moieties are unsubstituted or substituted with halogen or CN;
In another preferred embodiment, R^{3B} is H or C₁-C₆-alkyl;
In another preferred embodiment, R^{3B} is C₁-C₆-alkyl, which is unsubstituted;
In another preferred embodiment, R^{3B} is C₁-C₆-alkyl, which is substituted with halogen or CN;
In another preferred embodiment, R^{3B} is CH₃;
In another preferred embodiment, R^{3B} is H;
In another preferred embodiment, R^{3B} is H or CH₃;
In one preferred embodiment, D is DA, DB, DC, DD, DE, or DF;
In another preferred embodiment, D is DA, DB, DC, DD, or DE;
In another preferred embodiment, D is DA, DB, DC, or DD;
In another preferred embodiment, D is DA or DB;
In another preferred embodiment, D is DC or DD;
In another preferred embodiment, D is DE or DF;
In another preferred embodiment, D is DA;
In another preferred embodiment, D is DB;
In another preferred embodiment, D is DC;
In another preferred embodiment, D is DD;
In another preferred embodiment, D is DE;
In another preferred embodiment, D is DA, DC, or DE;
In another preferred embodiment, D is DB, DD, or DF;
In one preferred embodiment, W is S;
In another preferred embodiment, W is O;
In one preferred embodiment, R⁴ is H or C₁-C₆-alkyl, wherein the alkyl is unsubstituted or substituted with halogen or CN;
In another preferred embodiment, R⁴ is H;
In another preferred embodiment, R⁴ is C₁-C₆-alkyl, which is unsubstituted or substituted with halogen or CN;
In one preferred embodiment, R⁵ is H or C₁-C₆-alkyl, wherein the alkyl moieties are unsubstituted or substituted with —O—(C═O)—C₁-C₆-alkoxy (i.e. —O—(C═O)—O—C₁-C₆-alkyl), —O—(C═O)—C₁-C₆-alkyl or CN;
In another preferred embodiment, R⁵ is C₁-C₆-alkyl, for example methyl, which is unsubstituted or substituted with —O—(C═O)—C₁-C₆-alkoxy or —O—(C═O)—C₁-C₆-alkyl;
In another preferred embodiment, R⁵ is H;
In one preferred embodiment, E is a 5- or 6-membered carbocyclic group, wherein 1 or 2 CH₂ moieties of the carbocyclic group may be replaced by a carbonyl group, O, or S, wherein the carbocyclic group is unsubstituted or substituted with R¹⁰;
In another preferred embodiment, E is a 5- or 6-membered carbocyclic group, wherein 1 or 2 CH₂ moieties of the carbocyclic group may be replaced by a carbonyl group, or S, wherein the carbocyclic group is unsubstituted or substituted with R¹⁰;
In another preferred embodiment, E is a 5- or 6-membered carbocyclic group, wherein 1 or 2 CH₂ moieties of the carbocyclic group may be replaced by a carbonyl group, wherein the carbocyclic group is unsubstituted or substituted with R¹⁰;
In another preferred embodiment, E is a 5- or 6-membered carbocyclic group, wherein 1 or 2 CH₂ moieties of the carbocyclic group may be replaced by a carbonyl group, wherein the carbocyclic group is unsubstituted;
In another preferred embodiment, E is a 5-membered carbocyclic group, wherein 1 or 2 CH₂ moieties of the carbocyclic group may be replaced by a carbonyl group, wherein the carbocyclic group is unsubstituted or substituted with R¹⁰;
In another preferred embodiment, E is a 6-membered carbocyclic group, wherein 1 or 2 CH₂ moieties of the carbocyclic group may be replaced by a carbonyl group, wherein the carbocyclic group is unsubstituted or substituted with R¹⁰;
In another preferred embodiment, E is E1 or E2, which is unsubstituted or substituted with R¹⁰;

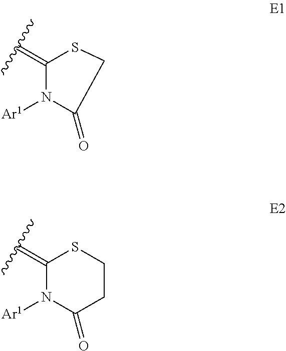

In one preferred embodiment, Ar¹ is phenyl which is unsubstituted or substituted with R^{Ar1};
In another preferred embodiment, Ar¹ is 5- or 6-membered hetaryl, which is unsubstituted or substituted with R^{Ar1};
In another preferred embodiment, Ar¹ is phenyl, pyrimidinyl, pyridazinyl, thiophenyl, thiazolyl, or pyridyl, which are unsubstituted or substituted with R^{Ar1};
In another preferred embodiment, Ar¹ is phenyl, which is substituted with R^{Ar1}.
In various preferred embodiments, Ar¹ is phenyl, which is substituted with R^{Ar1} in the 2-position, the 3-position or in the 2- and 5-position.
In one preferred embodiment, R^{Ar1} is halogen, SF₅, OH, CN, NR⁸R⁹, C₁-C₆-alkyl, C₁-C₆-alkoxy, C₃-C₆-cycloalkyl, C₃-C₆-cycloalkoxy, or C₁-C₆-alkyl-C₁-C₆-alkoxy (i.e. C₁-C₆-alkyl-O—C₁-C₆-alkyl), wherein the alkyl, alkoxy, cycloalkyl and cycloalkoxy moieties are unsubstituted or substituted with halogen or CN;
In another preferred embodiment, R^{Ar1} is halogen, CN, C₁-C₆-alkyl, C₁-C₆-alkoxy, C₁-C₆-alkoxy-C₁-C₄-alkyl, C₃-C₆-cycloalkyl, wherein the alkyl, alkoxy, and cycloalkyl moieties are unsubstituted or substituted with halogen,
In another preferred embodiment, R^{Ar1} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen,
In another preferred embodiment, R^{Ar1} is CN, C₁-C₆-alkyl, C₃-C₆-cycloalkyl, NR⁸R⁹ wherein alkyl and cycloalkyl moieties are unsubstituted or substituted with halogen;
In another preferred embodiment, R^{Ar1} is CN, C₁-C₆-alkyl, C₃-C₆-cycloalkyl, or NR⁸R⁹;
In another preferred embodiment, R^{Ar1} is C₁-C₆-alkyl, C₃-C₆-cycloalkyl, C₁-C₆-alkoxy-C₁-C₄-alkyl, halogen, or NR⁸R⁹, wherein alkyl and cycloalkyl moieties are unsubstituted or substituted with halogen;
In another preferred embodiment, R^{Ar1} is methyl, isopropyl, cyclopropyl, CH₂—O—CH₂—CF₃, F, or N(CH₃)₂;
In another preferred embodiment, R^{Ar1} is halogen, CN, C₁-C₆-alkyl, wherein the alkyl moieties are unsubstituted or substituted with halogen;
In another preferred embodiment, R^{Ar1} is halogen or C₁-C₆-alkyl, wherein the alkyl moieties are unsubstituted or substituted with halogen;
In another preferred embodiment, R^{Ar1} is halogen or C₁-C₆-alkyl;
In another preferred embodiment, R^{Ar1} is halogen;
In another preferred embodiment, R^{Ar1} is C₁-C₆-alkyl;
In another preferred embodiment, R^{Ar1} is halogen or CN,
In one preferred embodiment, R⁸ and R⁹ are, identical or different, H or C₁-C₆-alkyl;
In another preferred embodiment, Ar¹ is phenyl which is unsubstituted or substituted with R^{Ar1} and wherein
R^{Ar1} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen.

In another preferred embodiment, Ar¹ is selected from Ar¹-1 to Ar¹-19 as shown in Table Ar¹,

TABLE Ar1

| Ar¹ | Structure |
|---|---|
| Ar¹-1 | 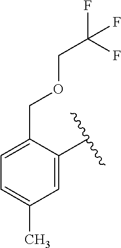 [SMILES: *c1cc(C)ccc1COCC(F)(F)F] |
| Ar¹-2 | 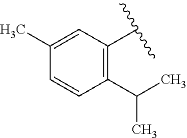 [SMILES: *c1cc(C)ccc1C(C)C] |
| Ar¹-3 | 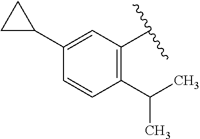 [SMILES: *c1cc(C2CC2)ccc1C(C)C] |
| Ar¹-4 | 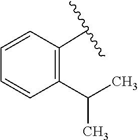 [SMILES: *c1ccccc1C(C)C] |
| Ar¹-5 | 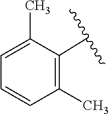 [SMILES: *c1c(C)cccc1C] |
| Ar¹-6 | 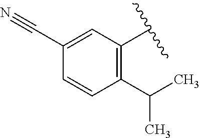 [SMILES: *c1cc(C#N)ccc1C(C)C] |
| Ar¹-7 | 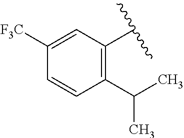 [SMILES: *c1cc(C(F)(F)F)ccc1C(C)C] |
| Ar¹-8 | 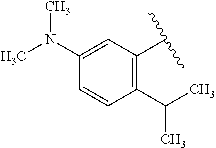 [SMILES: *c1cc(N(C)C)ccc1C(C)C] |
| Ar¹-9 | 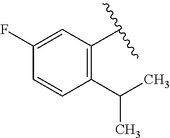 [SMILES: *c1cc(F)ccc1C(C)C] |
| Ar¹-10 | 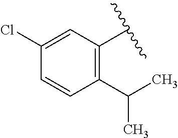 [SMILES: *c1cc(Cl)ccc1C(C)C] |
| Ar¹-11 | 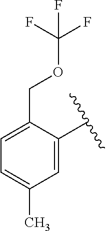 [SMILES: *c1cc(C)ccc1COC(F)(F)F] |
| Ar¹-12 | 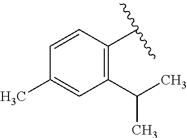 [SMILES: *c1ccc(C)cc1C(C)C] |
| Ar¹-13 | 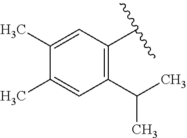 [SMILES: *c1cc(C)c(C)cc1C(C)C] |
| Ar¹-14 | 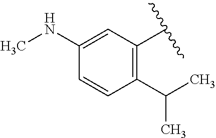 [SMILES: *c1cc(NC)ccc1C(C)C] |
| Ar¹-15 | 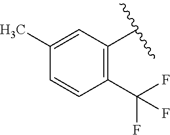 [SMILES: *c1cc(C)ccc1C(F)(F)F] |
| Ar¹-16 | 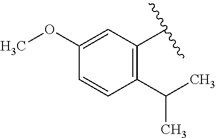 [SMILES: *c1cc(OC)ccc1C(C)C] |
| Ar¹-17 | 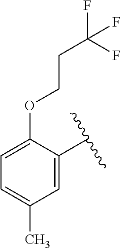 [SMILES: *c1cc(C)ccc1OCCC(F)(F)F] |
| Ar¹-18 | 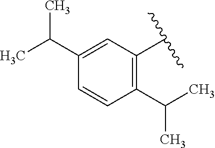 [SMILES: *c1cc(C(C)C)ccc1C(C)C] |
| Ar¹-19 | 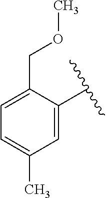 [SMILES: *c1cc(C)ccc1COC] |

In another preferred embodiment, Ar¹ is selected from Ar¹-2 to Ar¹-8;
In another preferred embodiment, Ar¹ is selected from Ar¹-1 to Ar¹-7;
In another preferred embodiment, Ar¹ is Ar¹-2 to Ar¹-5.
In another preferred embodiment, Ar¹ is selected from Ar¹-1 to Ar¹-5, Ar¹-8, Ar¹-9 and Ar¹-15.
In another preferred embodiment, Ar¹ is Ar¹-2 or Ar¹-4.
In another preferred embodiment Ar¹ is selected from Ar¹-8, Ar¹-9 and Ar¹-15.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X1 or X2;
A is N or CR^{2A};
R^{2A} is H, halogen, or C₁-C₆-alkyl, preferably H, Cl or CH₃;
R^{3A} is H, C₃-C₆-cycloalkyl, or C₁-C₆-alkyl, wherein the alkyl is unsubstituted or substituted with halogen, CN or C₁-C₆-alkoxy, preferably R^{3A} is H, CH₃ or cyclopropyl;
R^{3B} is H or C₁-C₆-alkyl, preferably CH₃;
B¹ is CR^{B1}, B² is CR^{B2}, and B³ is CR^{B3};
R^{B1}, R^{B2}, R^{B3}, and RB⁴ independently of each other are H, halogen, C₁-C₆-alkyl, or CN, preferably H, halogen or C₁-C₆-alkyl, more preferably H, F, Cl, Br, or CH₃;
D is DA, DB, DC, DD, or DE;
W is O or S;

R⁴ is H;
R⁵ is C₁-C₆-alkyl, which is unsubstituted or substituted with —O—(C═O)—C₁-C₆-alkoxy or —O—(C═O)—C₁-C₆-alkyl, preferably CH₂—O—C(═O)—C₁-C₆-alkyl or CH₂—O—C(═O)—O—C₁-C₆-alkyl;
Ar¹ is phenyl which is unsubstituted or substituted with R^{Ar1};
R^{Ar1} is halogen, CN, C₁-C₆-alkyl, C₃-C₆-cycloalkyl, C₁-C₆-alkyl-O—C₁-C₆-alkyl, or NR⁸R⁹ wherein alkyl and cycloalkyl moieties are unsubstituted or substituted with halogen, preferably R^{Ar1} is methyl, isopropyl, cyclopropyl, —N(CH₃)₂, —CF₃, —CH₂—O—CH₂—CF₃, or —F;
R⁸ and R⁹ are, identical or different, H or C₁-C₆-alkyl.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X1 or X2;
A is N or CR^{2A};
R^{2A} is H, halogen, or C₁-C₆-alkyl, preferably H, Cl or CH₃;
R^{3A} is H, C₃-C₆-cycloalkyl, or C₁-C₆-alkyl, wherein the alkyl is unsubstituted or substituted with halogen, CN or C₁-C₆-alkoxy, preferably R^{3A} is H, CH₃ or cyclopropyl;
R^{3B} is H or C₁-C₆-alkyl, preferably CH₃;
B¹ is CR^{B1}, B² is CR^{B2}, and B³ is CR^{B3};
R^{B1}, R^{B2}, R^{B3}, and RB⁴ independently of each other are H, halogen, C₁-C₆-alkyl, or CN, preferably H, halogen or C₁-C₆-alkyl, more preferably H, F, Cl, Br, or CH₃;
D is DA, DB, DC or DD; preferably DC or DD;
W is O or S, preferably S;
R⁴ is H;
R⁵ is C₁-C₆-alkyl, which is unsubstituted or substituted with —O—(C═O)—C₁-C₆-alkoxy or —O—(C═O)—C₁-C₆-alkyl, preferably CH₂—O—C(═O)—C₁-C₆-alkyl or CH₂—O—C(═O)—O—C₁-C₆-alkyl;
Ar¹ is phenyl which is unsubstituted or substituted with R^{Ar1};
R^{Ar1} is halogen, CN, C₁-C₆-alkyl, C₃-C₆-cycloalkyl, C₁-C₆-alkyl-O—C₁-C₆-alkyl, or NR⁸R⁹ wherein alkyl and cycloalkyl moieties are unsubstituted or substituted with halogen, preferably R^{Ar1} is methyl, isopropyl, cyclopropyl, —N(CH₃)₂, —CF₃, —CH₂—O—CH₂—CF₃, or —F;
R⁸ and R⁹ are, identical or different, H or C₁-C₆-alkyl.

In another preferred embodiment of compound of formula I, wherein
n is 1 or 2;
R^{1A}, R^{1B}, R^{1C} and R^{1D} independently of each other are selected from H, Cl, F, Br, CN, CF₃, and OCF₃;
X is X1 or X2;
A is N, C(CH₃), or CH;
R^{2A} is H, Cl, or CH₃;
R^{3A} is C₁-C₆-alkyl or cyclopropyl;
R^{3B} is CH₃;
B¹ is CR^{B1}, B² is CR^{B2}, and B³ is CR^{B3};
R^{B1}, R^{B2}, R^{B3}, and RB⁴ independently of each other are H, halogen, or CN;
D is DA, DB, DC, DD, or DE;
W is O or S, preferably S;
R⁴ is H;
R⁵ is C₁-C₆-alkyl, which is unsubstituted or substituted with —O—(C═O)—C₁-C₆-alkoxy or —O—(C═O)—C₁-C₆-alkyl, preferably CH₂—O—C(═O)—C₁-C₆-alkyl or CH₂—O—C(═O)—O—C₁-C₆-alkyl;
Ar¹ is selected from Ar¹-1 to Ar¹-9 and Ar¹-15, or from Ar¹-2 to Ar¹-8.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X1;
A is N, C(C₁-C₆-alkyl), or CH;
R^{3A} is H or C₁-C₆-alkyl, wherein the alkyl is unsubstituted or substituted with halogen, CN or C₁-C₆-alkoxy;
B¹ is N or CR^{B1}, B² is CR^{B2}, and B³ is CR^{B3};
R^{B1}, R^{B2}, R^{B3}, and RB⁴ independently of each other are H, halogen, or CN;
D is DA or DB;
W is S or O, preferably S;
R⁴ is H or C₁-C₆-alkyl;
Ar¹ is phenyl which is unsubstituted or substituted with R^{Ar1};
R^{Ar1} is CN, C₁-C₆-alkyl, C₃-C₆-cycloalkyl, NR⁸R⁹ wherein alkyl and cycloalkyl moieties are unsubstituted or substituted with halogen;
R⁸ and R⁹ are, identical or different, H or C₁-C₆-alkyl.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X1;
A is N, C(C₁-C₆-alkyl), or CH;
R^{3A} is H or C₁-C₆-alkyl, wherein the alkyl is unsubstituted or substituted with halogen, CN or C₁-C₆-alkoxy;
B¹ is N or CR^{B1}, B² is CR^{B2}, and B³ is CR^{B3};
R^{B1}, R^{B2}, R^{B3}, and RB⁴ independently of each other are H, halogen, or CN;
D is DC or DD;
Ar¹ is phenyl which is unsubstituted or substituted with R^{Ar1};
R^{Ar1} is CN, C₁-C₆-alkyl, C₃-C₆-cycloalkyl, NR⁸R⁹ wherein alkyl and cycloalkyl moieties are unsubstituted or substituted with halogen;
R⁸ and R⁹ are, identical or different, H or C₁-C₆-alkyl.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X1;
A is N, C(C₁-C₆-alkyl), or CH;
R^{3A} is H or C₁-C₆-alkyl, wherein the alkyl is unsubstituted or substituted with halogen, CN or C₁-C₆-alkoxy;
B¹ is N or CR^{B1}, B² is CR^{B2}, and B³ is CR^{B3};
R^{B1}, R^{B2}, R^{B3}, and RB⁴ independently of each other are H, halogen, or CN;
D is DE or DF;
R⁴ is H or C₁-C₆-alkyl;
R⁵ is C₁-C₆-alkyl, which is unsubstituted or substituted with —O—(C═O)—C₁-C₆-alkoxy or —O—(C═O)—C₁-C₆-alkyl;
Ar¹ is phenyl which is unsubstituted or substituted with R^{Ar1};
R^{Ar1} is CN, C₁-C₆-alkyl, C₃-C₆-cycloalkyl, NR⁸R⁹ wherein alkyl and cycloalkyl moieties are unsubstituted or substituted with halogen;
R⁸ and R⁹ are, identical or different, H or C₁-C₆-alkyl.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X2;
R^{2A} is H, halogen, or C₁-C₆-alkyl;
R^{3B} is H or C₁-C₆-alkyl, wherein the alkyl is unsubstituted or substituted with halogen or CN;
B¹ is N or CR^{B1}, B² is CR^{B2}, and B³ is CR^{B3};
R^{B1}, R^{B2}, R^{B3}, and RB⁴ independently of each other are H, halogen, or CN;
D is DA or DB;
W is S or O, preferably S;
R⁴ is H or C₁-C₆-alkyl;
Ar¹ is phenyl which is unsubstituted or substituted with R^{Ar1};
R^{Ar1} is CN, C₁-C₆-alkyl, C₃-C₆-cycloalkyl, or NR⁸R⁹ wherein alkyl and cycloalkyl moieties are unsubstituted or substituted with halogen;
R⁸ and R⁹ are, identical or different, H or C₁-C₆-alkyl.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl and C₁-C₆-haloalkoxy;
X is X2;
R^{2A} is H, halogen, or C₁-C₆-alkyl;
R^{3B} is H or C₁-C₆-alkyl, wherein the alkyl is unsubstituted or substituted with halogen or CN;
B¹ is N or CR^{B1}, B² is CR^{B2}, and B³ is CR^{B3};
R^{B1}, R^{B2}, R^{B3}, and RB⁴ independently of each other are H, halogen, or CN;
D is DC or DD;
Ar¹ is phenyl which is unsubstituted or substituted with R^{Ar1};
R^{Ar1} is CN, C₁-C₆-alkyl, C₃-C₆-cycloalkyl, NR⁸R⁹ wherein alkyl and cycloalkyl moieties are unsubstituted or substituted with halogen;
R⁸ and R⁹ are, identical or different, H or C₁-C₆-alkyl.

In another preferred embodiment of compound of formula I, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X2;
R^{2A} is H, halogen, or C₁-C₆-alkyl;
R^{3B} is H or C₁-C₆-alkyl, wherein the alkyl is unsubstituted or substituted with halogen or CN;
B¹ is N or CR^{B1}, B² is CR^{B2}, and B³ is CR^{B3};
R^{B1}, R^{B2}, R^{B3}, and RB⁴ independently of each other are H, halogen, or CN;
D is DE or DF;
R⁴ is H or C₁-C₆-alkyl;
R⁵ is C₁-C₆-alkyl, which is unsubstituted or substituted with —O—(C═O)—C₁-C₆-alkoxy or —O—(C═O)—C₁-C₆-alkyl;
Ar¹ is phenyl which is unsubstituted or substituted with R^{Ar1};
R^{Ar1} is CN, C₁-C₆-alkyl, C₃-C₆-cycloalkyl, NR⁸R⁹ wherein alkyl and cycloalkyl moieties are unsubstituted or substituted with halogen;
R⁸ and R⁹ are, identical or different, H or C₁-C₆-alkyl.

In any of the afore-mentioned preferred embodiments of the compound of formula I, R^{1A} and R^{1C} may be H and R^{1B} and R^{1D} are independently of each other selected from H, halogen, CN, C₁-C₆-haloalkyl, and C₁-C₆-haloalkoxy.

In any of the afore-mentioned preferred embodiments of the compound of formula I, R^{1A}, R^{1B} and R^{1C} may be H and R^{1D} may be halogen.

In any of the afore-mentioned preferred embodiments of the compound of formula I, R^{1A}, R^{1C} and R^{1D} may be H and R^{1B} is selected from halogen, for example F or Cl, CN, C₁-C₆-haloalkyl, for example CF₃, and C₁-C₆-haloalkoxy, for example OCF₃.

In another preferred embodiment, compounds of formula I are selected from compounds of formulae A.1 to A.120, wherein the variables are as defined herein above,

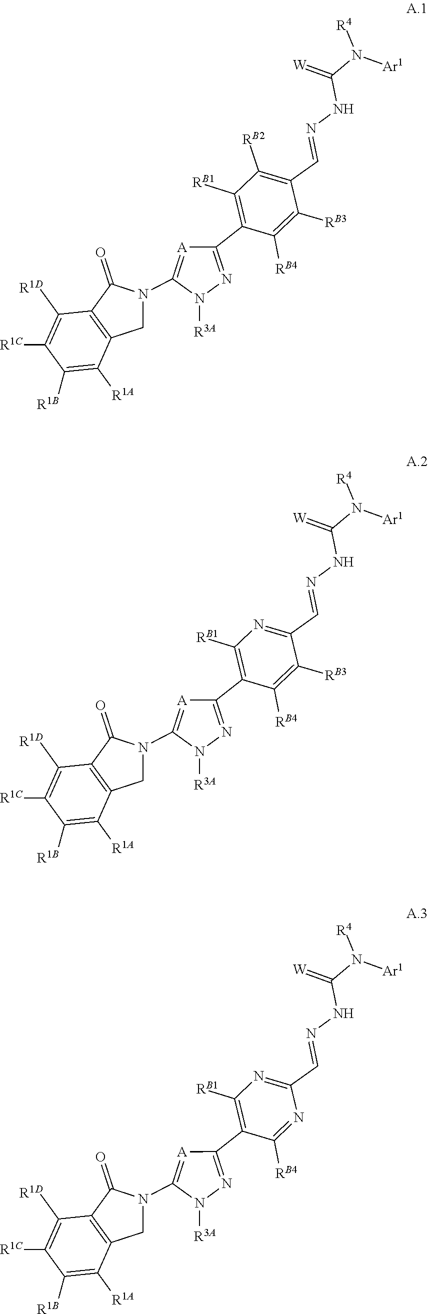

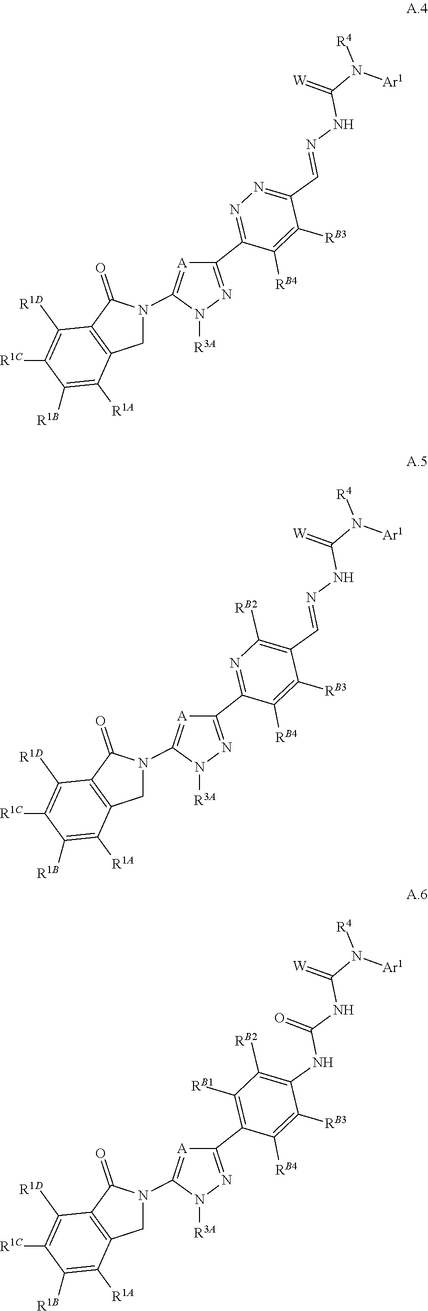

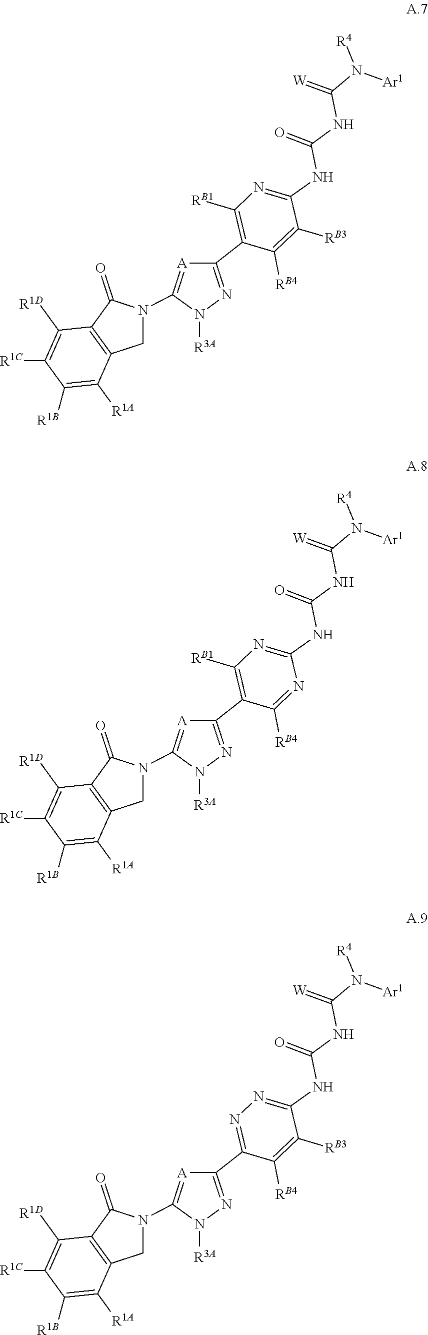

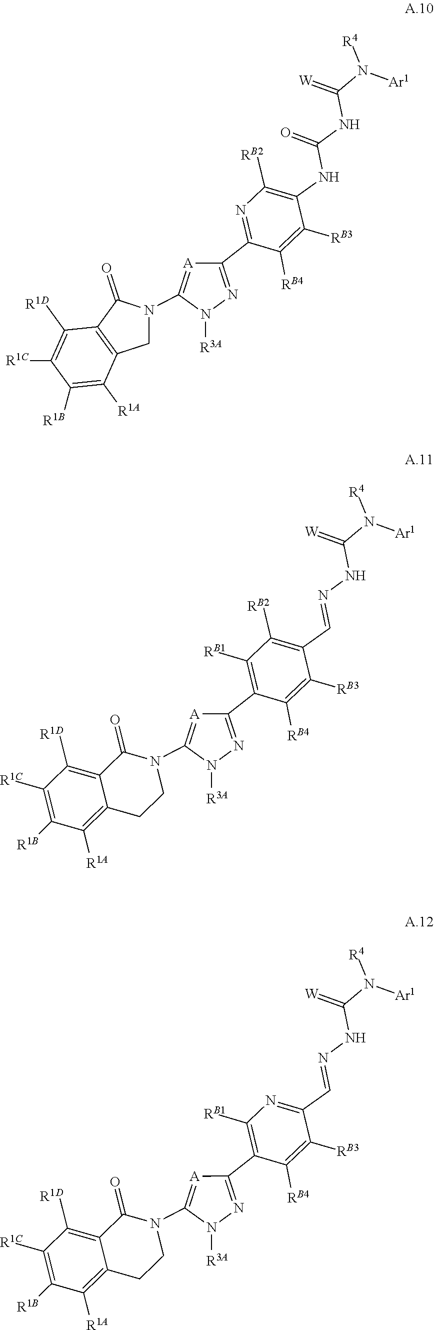

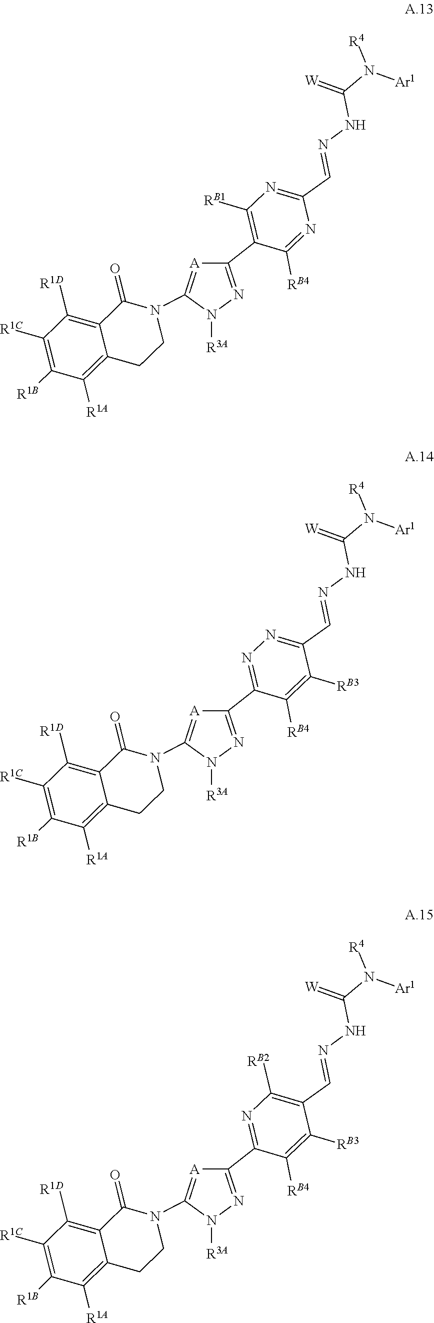

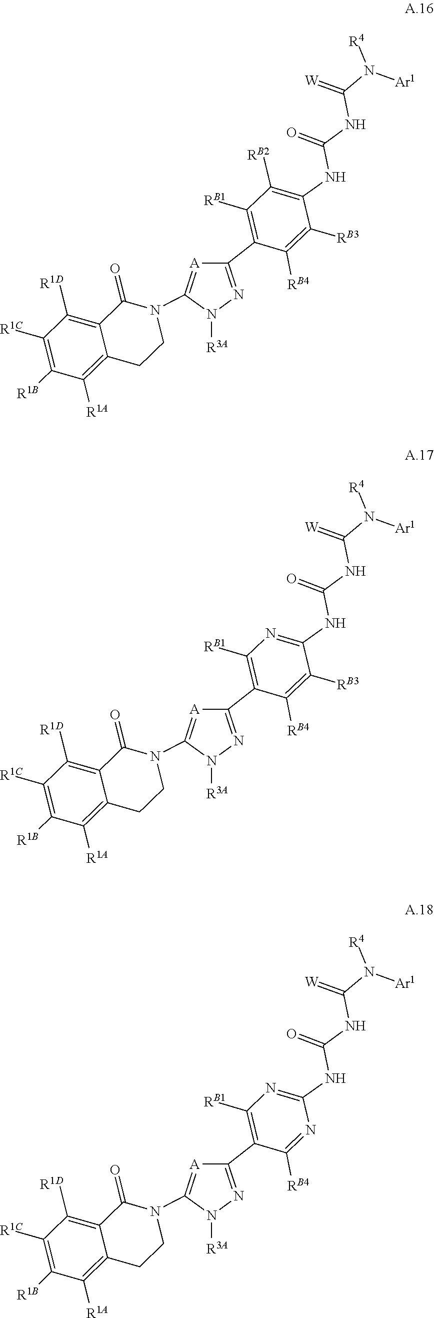

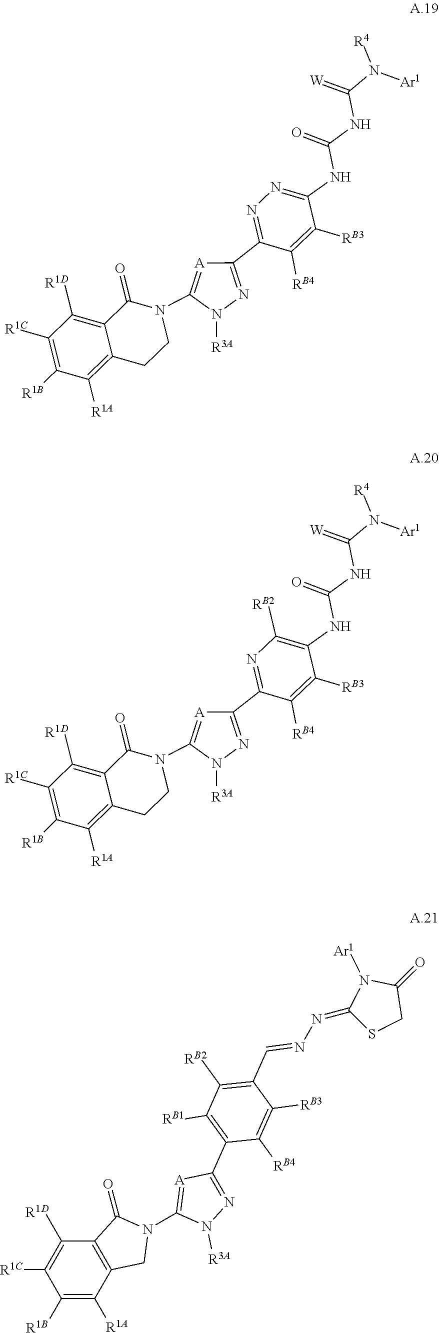

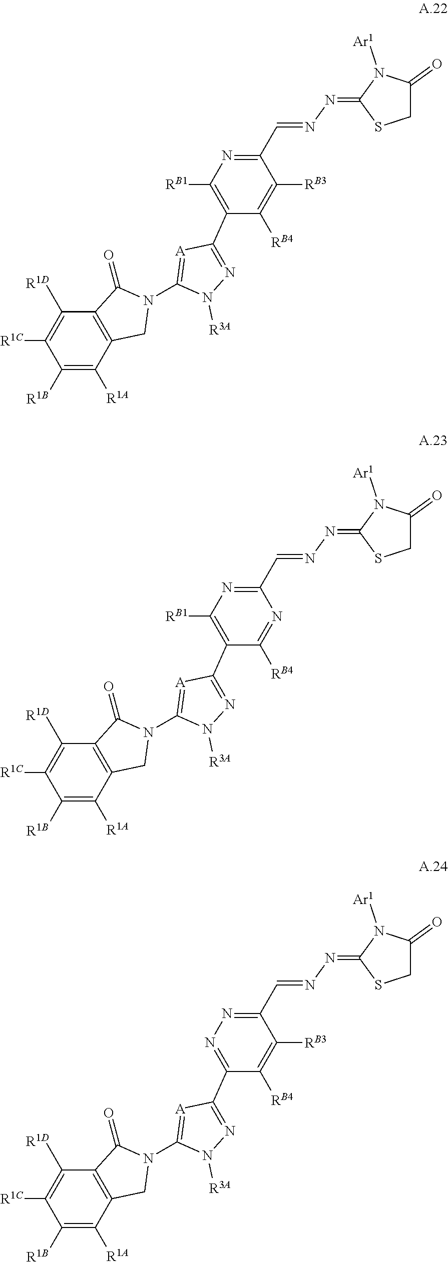

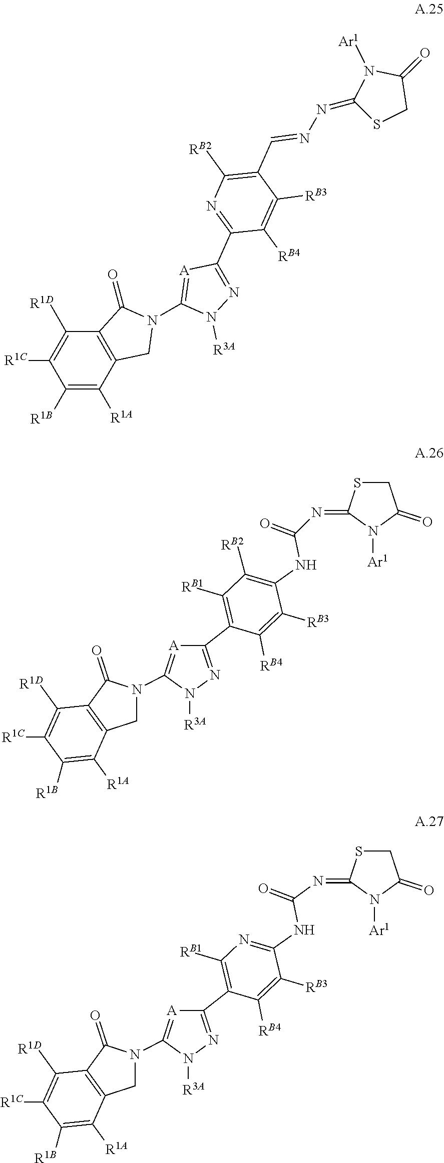

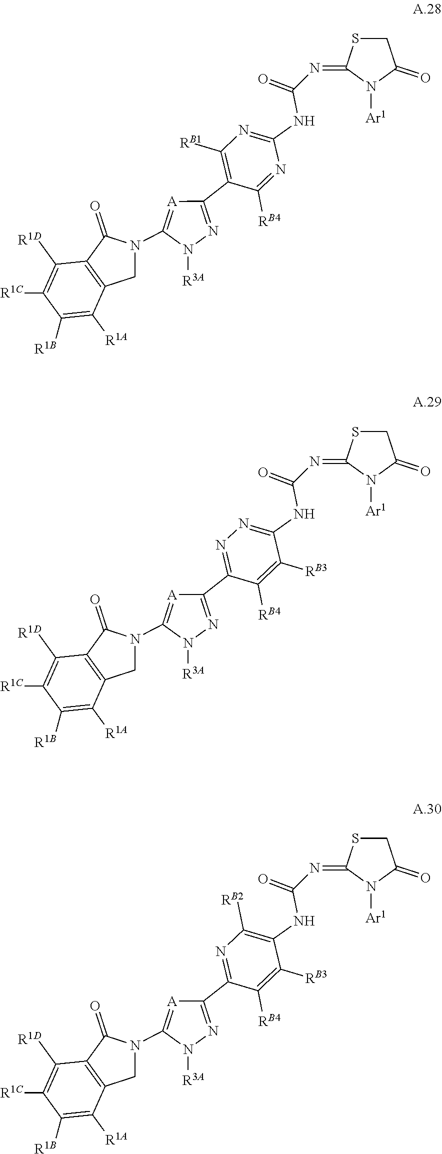

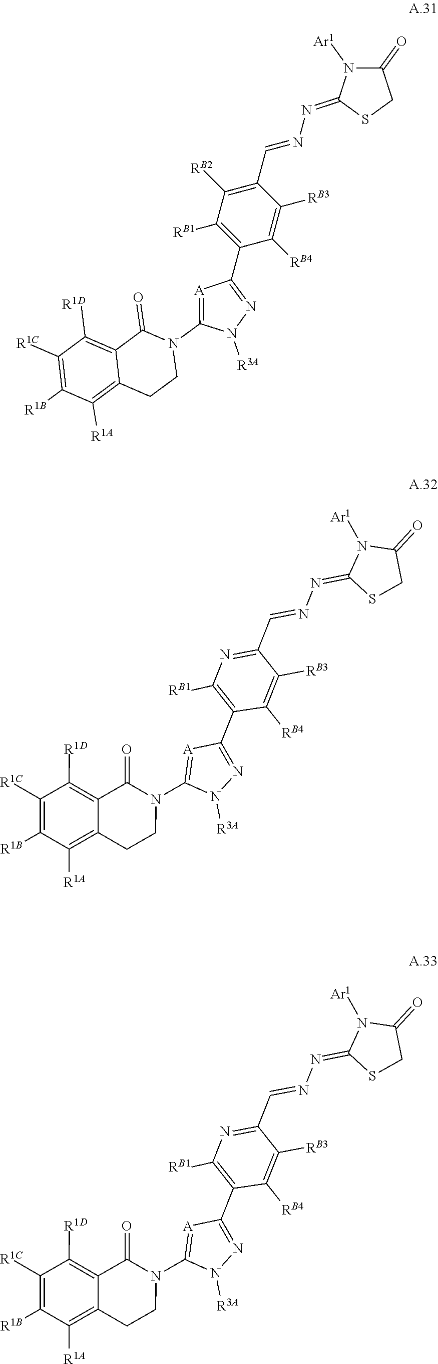

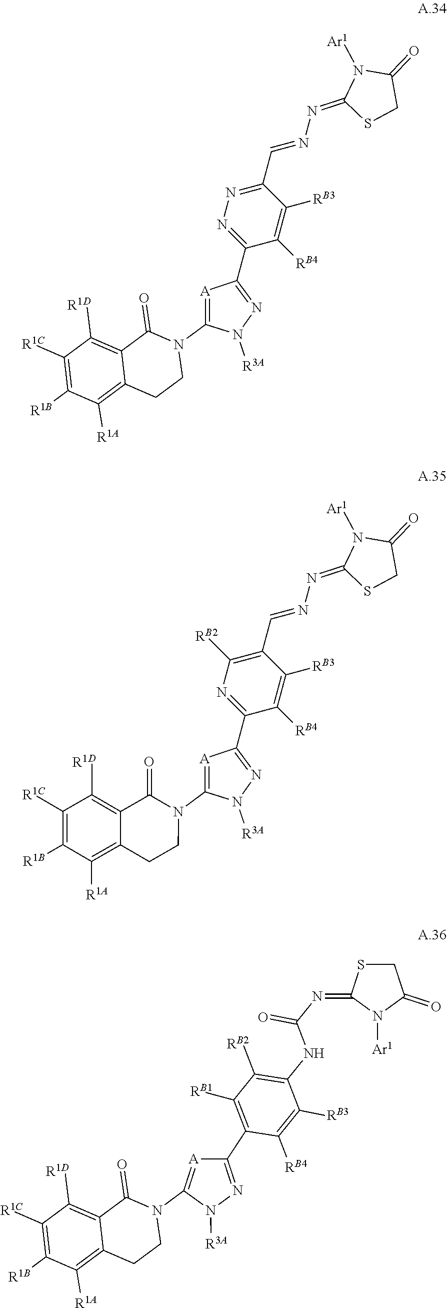

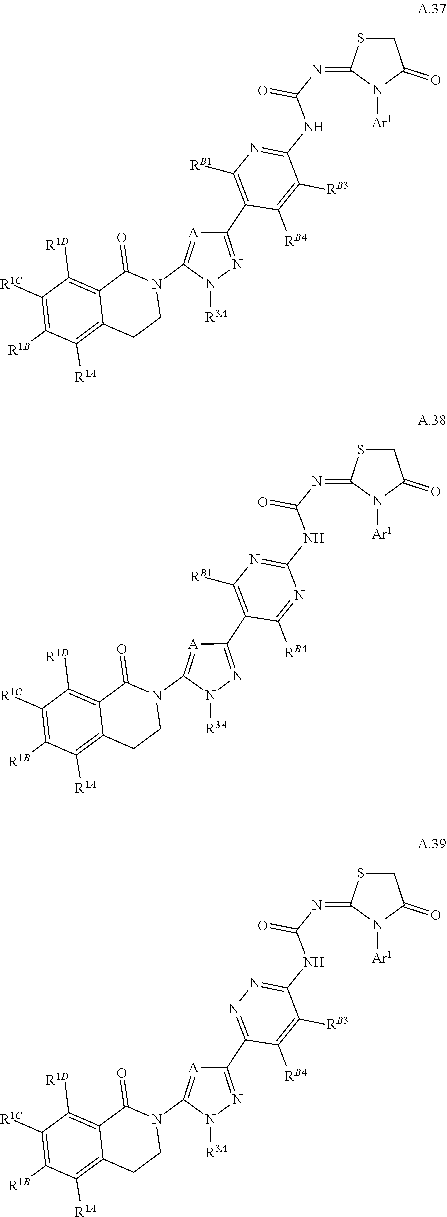

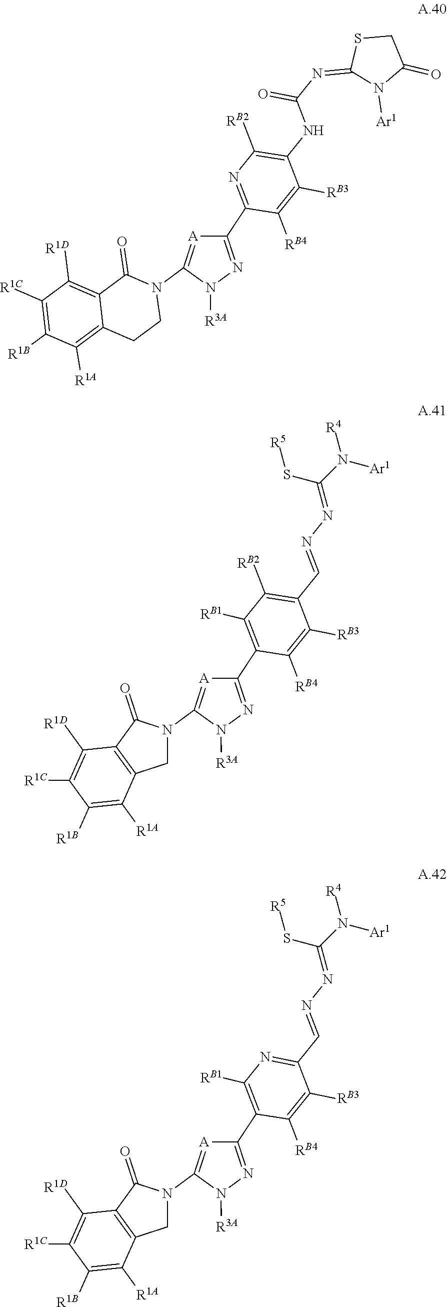

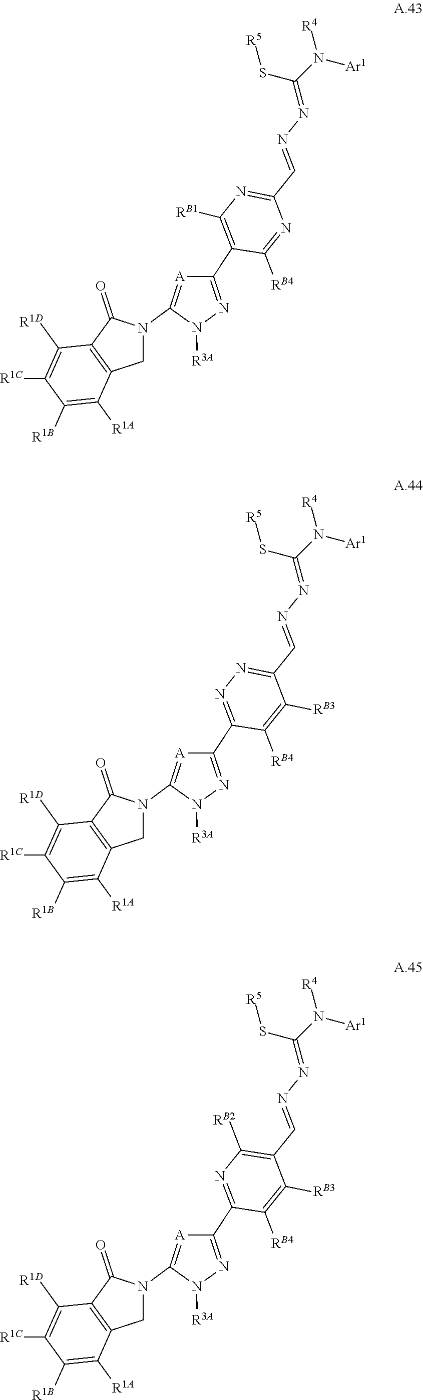

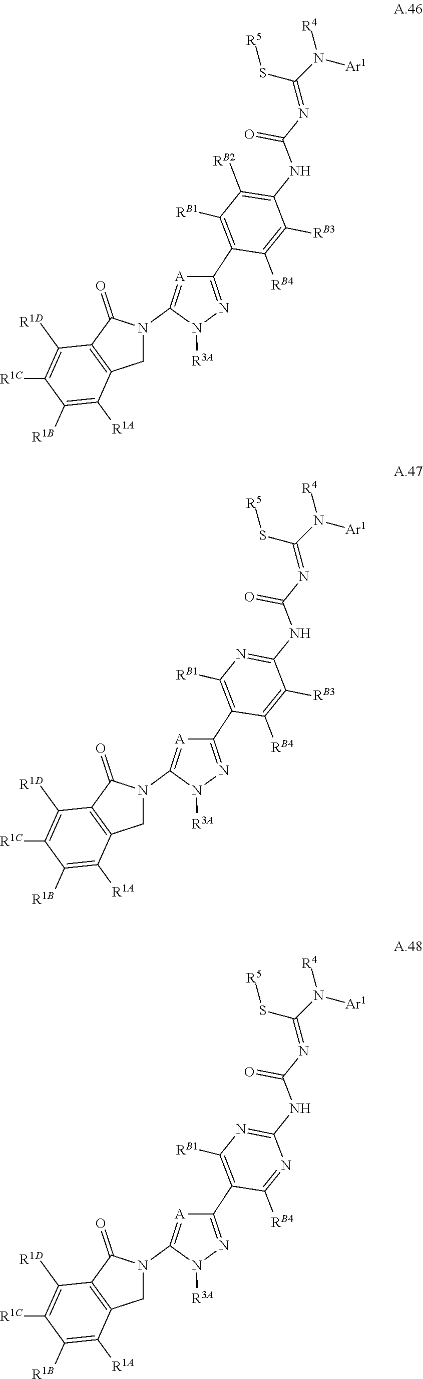

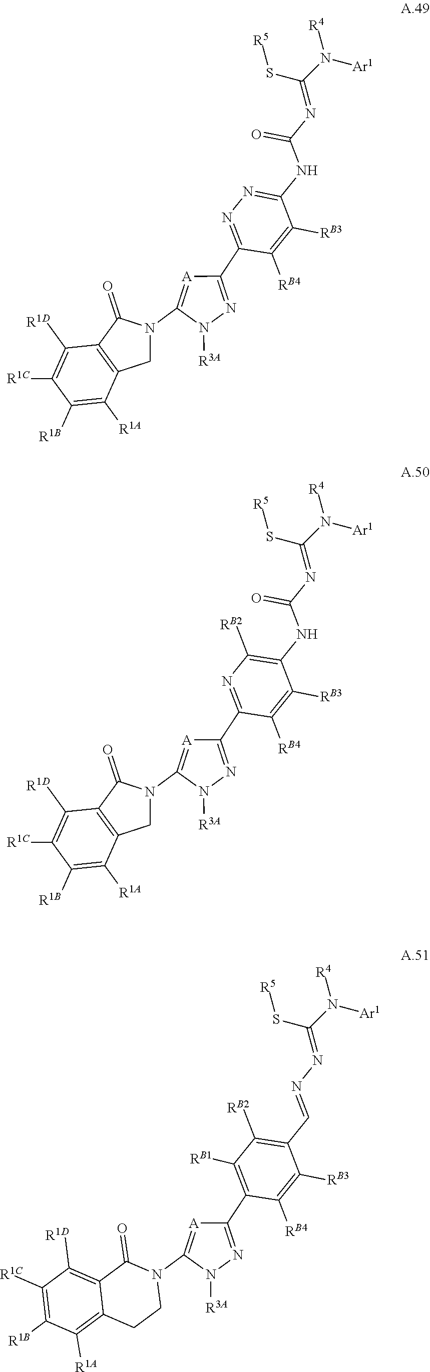

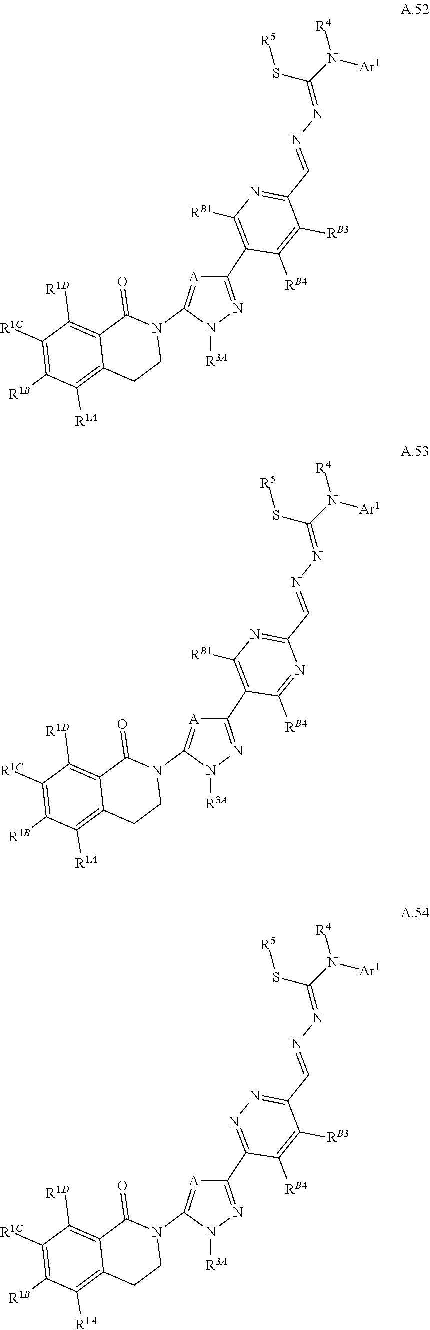

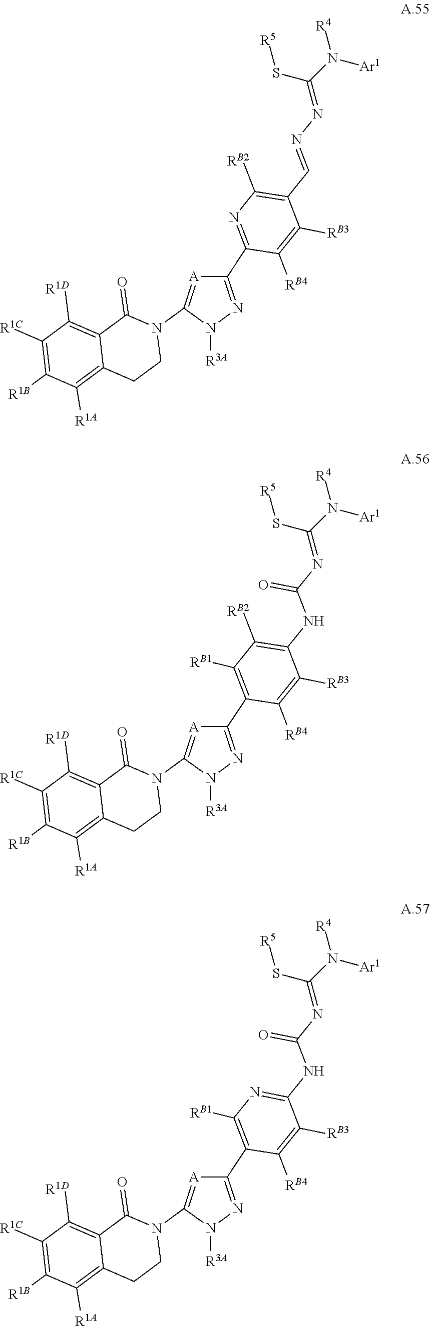

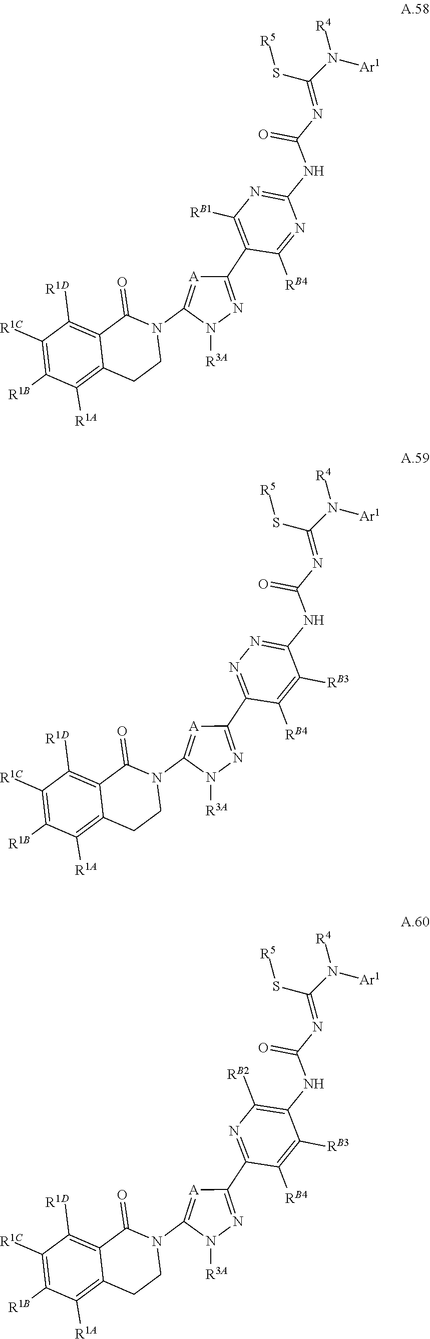

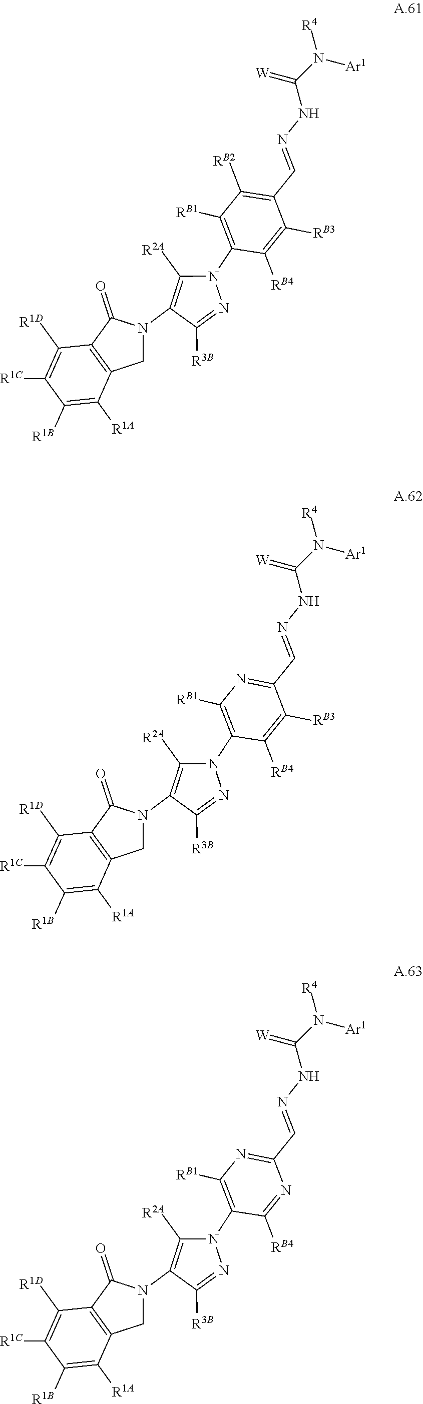

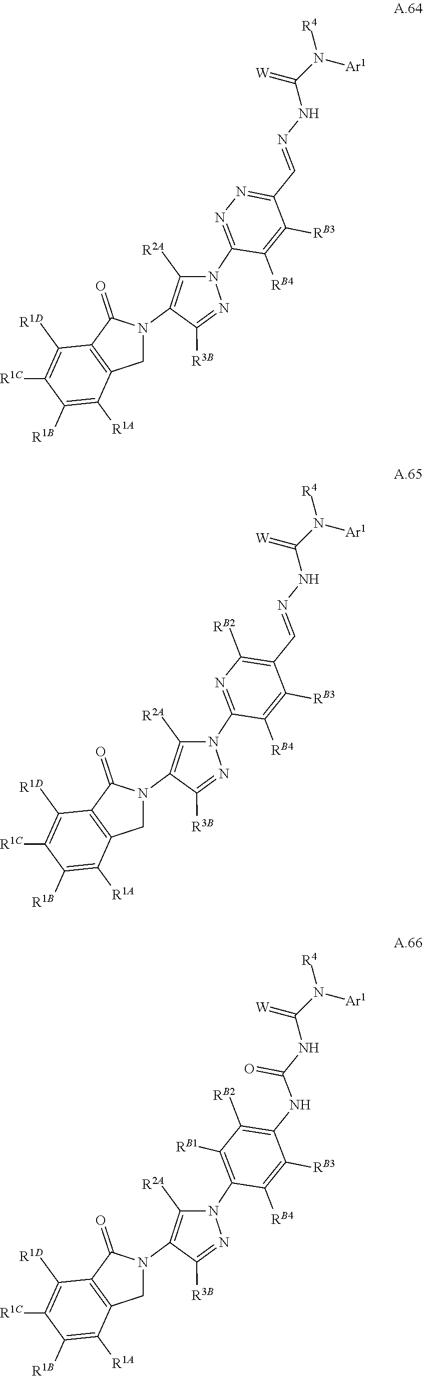

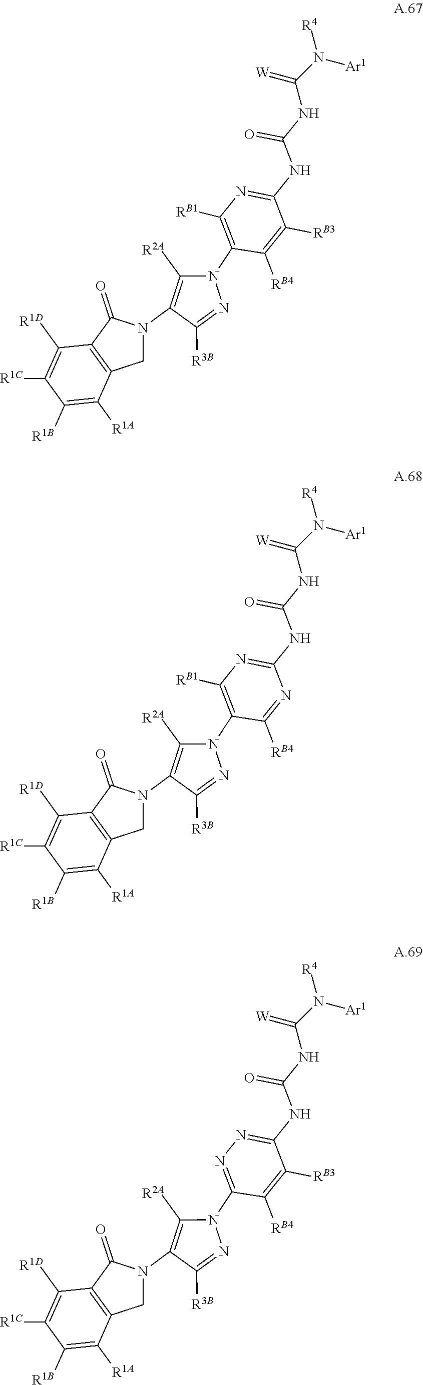

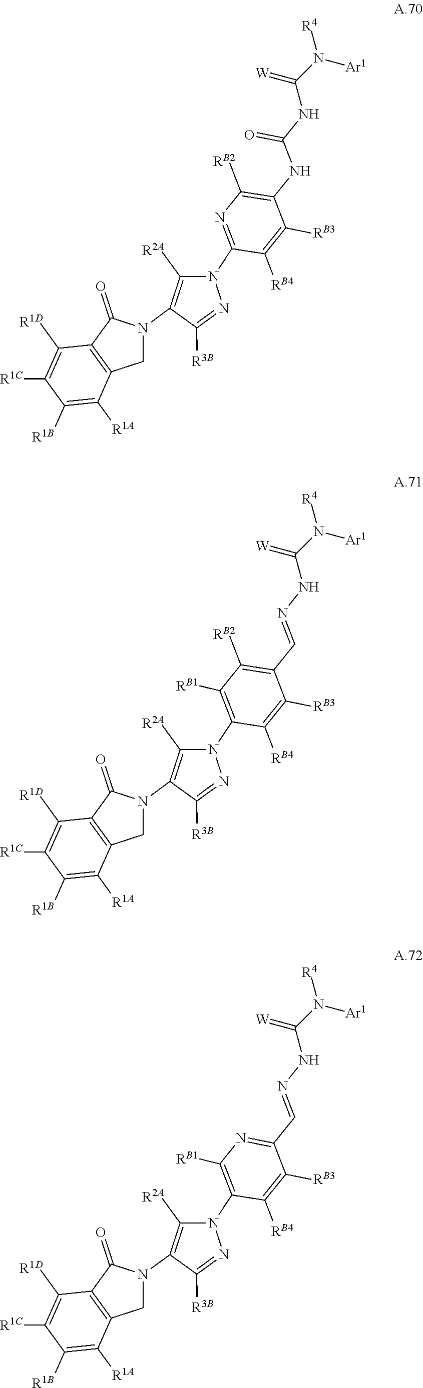

[chemical structure]

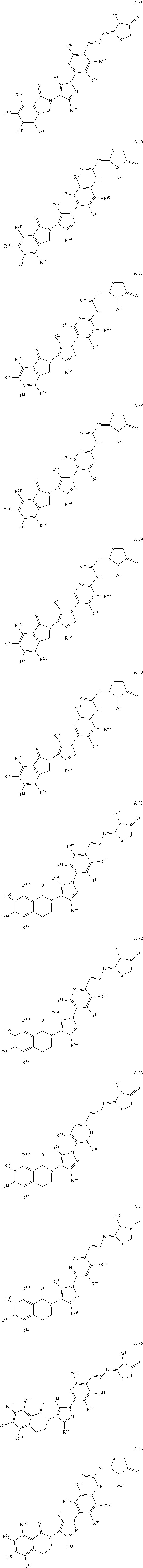

[chemical structure]

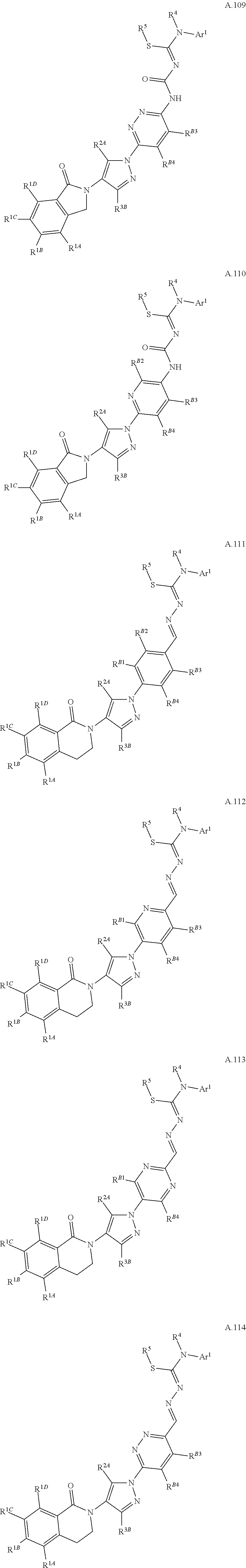

In another preferred embodiment, compounds of formula I are selected from compounds of formula A.1 to A.60;
In another preferred embodiment, compounds of formula I are selected from compounds of formula A.1 to A.20;
In another preferred embodiment, compounds of formula I are selected from compounds of formula A.1, A.6, A.11, and A.16;
In another preferred embodiment, compounds of formula I are selected from compounds of formula A.21 to A.40;
In another preferred embodiment, compounds of formula I are selected from compounds of formula A.21, A.26, A.31, and A.36;
In another preferred embodiment compounds of formula I are selected from compounds of formula A.41 to A.60
In another preferred embodiment, compounds of formula I are selected from compounds of formula A.41, A.46, A.51, and A.56;
In another preferred embodiment, compounds of formula I are selected from compounds of formula A.1, A.6, A.11, A.16, A.21, A.26, A.31, A.36, A.41, A.46, A.51, and A.56;
In another preferred embodiment, compounds of formula I are selected from compounds of formula A.21, A.26, A.31, and A.36;
In another preferred embodiment, compounds of formula I are selected from compounds of formula A.26, and A.36;
In another preferred embodiment, compounds of formula I are selected from compounds of formula A.61 to A.120;
In another preferred embodiment, compounds of formula I are selected from compounds of formula A.61 to A.80;
In another preferred embodiment, compounds of formula I are selected from compounds of formula A.61, A.66, A.71, and A.76;
In another preferred embodiment, compounds of formula I are selected from compounds of formula A.81 to A.100;
In another preferred embodiment, compounds of formula I are selected from compounds of formula A.81, A.86, A.91, and A.96;
In another preferred embodiment compounds of formula I are selected from compounds of formula A.101 to A.120
In another preferred embodiment, compounds of formula I are selected from compounds of formula A.101, A.106, A.111, and A.116;
In another preferred embodiment, compounds of formula I are selected from compounds of formula A.61, A.66, A.71, A.66, A.81, A.86, A.91, A.96, A.101, A.106, A.111, and A.116;
In another preferred embodiment, compounds of formula I are selected from compounds of formula A.81, A.86, A.91, and A.96;
In another preferred embodiment, compounds of formula I are selected from compounds of formula A.86, and A.96;
In another preferred embodiment, compounds of formula I are selected from compounds of formula A.1, A.6, A.11, A.16, A.21, A.26, A.31, A.36, A.41, A.46, A.51, A.56, A.61, A.66, A.71, A.76, A.81, A.86, A.91, A.96, A.101, A.106, and A.111;
In another preferred embodiment, compounds of formula I are selected from compounds of formula A.1, A.6, A.11, A.16, A.21, A.26, A.31, A.36, A.41, A.46, A.51, A.56, A.61, A.66, A.71, A.75, A.76, A.81, A.86, A.91, A.95, A.96, A.101, A.106, and A.111;
In another preferred embodiment, compounds of formula I are selected from compounds of formula A.21, A.26, A.31, A.36, A.81, A.86, A.91, and A.96;
In another preferred embodiment, compounds of formula I are selected from compounds of formula A.26, A.36, A.86, and A.96;
In another preferred embodiment compounds of formula I are selected from compounds of formula A.21, A.26, A.31, and A.36, wherein
R^{1A}, R^{1B}, R^{1C} and R^{1D} independently of each other are selected from H, halogen, CN, C₁-C₆-haloalkyl, and C₁-C₆-haloalkoxy;
A is N or CR^{2A};
R^{2A} is H, halogen, or C₁-C₆-alkyl;
R^{3A} is H or C₁-C₆-alkyl, wherein the alkyl is unsubstituted or substituted with halogen or C₁-C₆-alkoxy;
R^{B1}, R^{B2}, R^{B3}, and RB⁴ independently of each other are H, halogen, or CN;
Ar¹ is phenyl which is unsubstituted or substituted with R^{Ar1};
R^{Ar1} is CN, C₁-C₆-alkyl, C₃-C₆-cycloalkyl, NR⁸R⁹ wherein alkyl and cycloalkyl moieties are unsubstituted or substituted with halogen;
R⁸ and R⁹ are, identical or different, H or C₁-C₆-alkyl.

In another preferred embodiment compounds of formula I are selected from compounds of formula A.21, A.26, A.31, and A.36, wherein
R^{1A}, R^{1B}, R^{1C} and R^{1D} independently of each other are selected from H, Cl, F, Br, CN, CF₃, and OCF₃;
A is N, C(CH₃), or CH;
R^{3A} is C₁-C₆-alkyl;
R^{B1}, R^{B2}, R^{B3}, and RB⁴ independently of each other are H, halogen, or CN;
Ar¹ is Ar¹-2 to Ar¹-8;
In another preferred embodiment, compounds of formula I are selected from compounds of formula A.81, A.86, A.91, and A.96;
R^{1A}, R^{1B}, R^{1C} and R^{1D} independently of each other are selected from H, halogen, CN, C₁-C₆-haloalkyl, and C₁-C₆-haloalkoxy;
R^{2A} is H, halogen, or C₁-C₆-alkyl;
R^{3B} is H or C₁-C₆-alkyl, wherein the alkyl is unsubstituted or substituted with halogen or CN;
R^{B1}, R^{B2}, R^{B3}, and RB⁴ independently of each other are H, halogen, or CN;
Ar¹ is phenyl which is unsubstituted or substituted with R^{Ar1};
R^{Ar1} is CN, C₁-C₆-alkyl, C₃-C₆-cycloalkyl, NR⁸R⁹ wherein alkyl and cycloalkyl moieties are unsubstituted or substituted with halogen;
R⁸ and R⁹ are, identical or different, H or C₁-C₆-alkyl;
In another preferred embodiment, compounds of formula I are selected from compounds of formula A.81, A.86, A.91, and A.96;
R^{1A}, R^{1B}, R^{1C} and R^{1D} independently of each other are selected from H, Cl, F, CN, CF₃, and OCF₃;
R^{2A} is H, halogen, or C₁-C₆-alkyl;
R^{3B} is C₁-C₆-alkyl;
R^{B1}, R^{B2}, R^{B3}, and RB⁴ independently of each other are H, halogen, or CN;
R^{Ar1} is Ar¹-2 to Ar¹-8;
In another preferred embodiment, the compound of formula I is selected from the compounds of formulae I-1 to I-20, wherein the variables are as defined herein

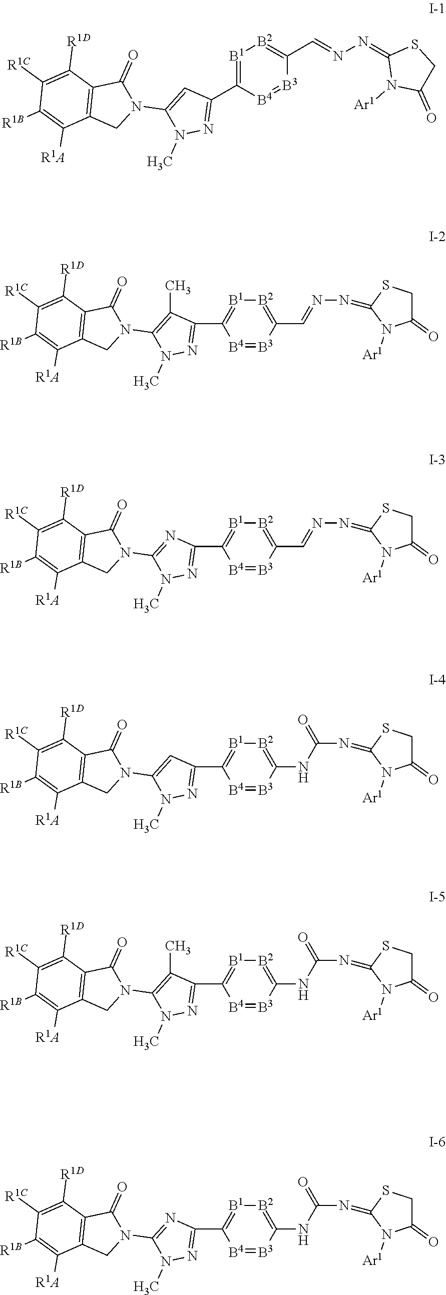

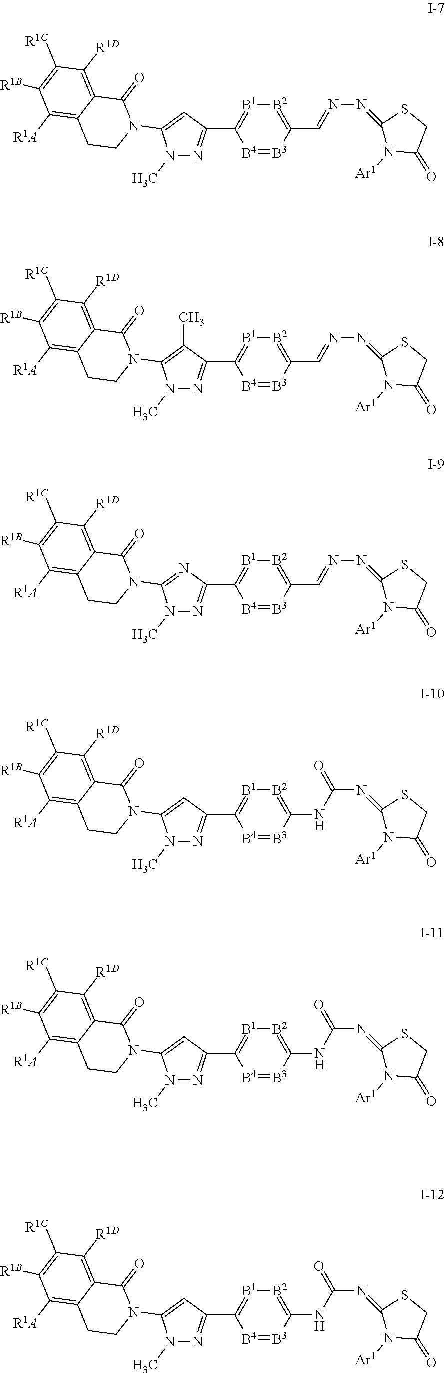

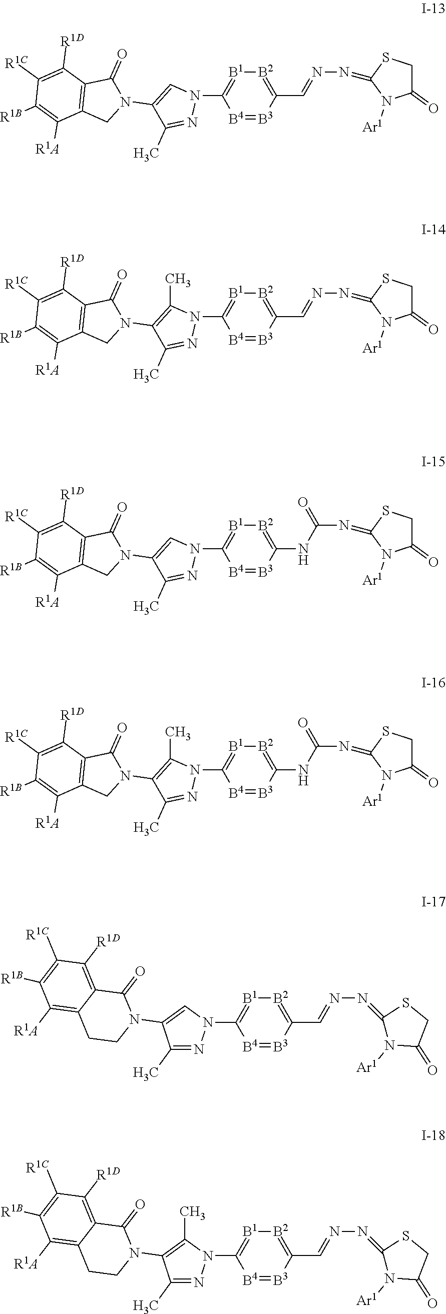

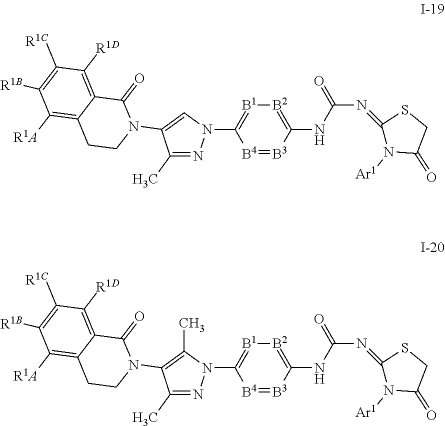

In another preferred embodiment, the compound of formula I is selected from the compounds of formulae I-1 to 1-12;
In another preferred embodiment, the compound of formula I is selected from the compounds of formulae I-13 to 1-20;
In another preferred embodiment, the compound of formula I is selected from the compounds of formulae I-1 to 1-6;
In another preferred embodiment, the compound of formula I is selected from the compounds of formulae I-4 and 1-5;
In another preferred embodiment, the compound of formula I is selected from the compounds of formulae I-4, 1-5, I-6, I-10, I-11, and I-12;
In another preferred embodiment, the compound of formula I is selected from the compounds of formulae 1-7 to 1-12;
In another preferred embodiment, the compound of formula I is selected from the compounds of formulae I-13 to I-16;
In another preferred embodiment, the compound of formula I is selected from the compounds of formulae I-17 to 1-20;
In another preferred embodiment, the compound of formula I is selected from the compounds of formulae I-15, I-16, I-19, and I-20;
In another preferred embodiment, the compound of formula I is compound of formula I-1 to 1-20, wherein
R^{1A}, R^{1B}, R^{1C} and R^{1D} independently of each other are selected from H, halogen, CN, C₁-C₆-haloalkyl, and C₁-C₆-haloalkoxy;
B¹ is N or CR^{B1};
B² is N or CR^{B2};
B³ is N or CR^{B3};
B⁴ is CR^{B4};
R^{B1}, R^{B2}, R^{B3}, and RB⁴ independently of each other are selected from H, halogen, CN, and C₁-C₆-alkyl, preferably CH₃;
Ar¹ is selected from Ar¹-2 to Ar¹-8;

Particular compounds of formula I are the compounds that are compiled in the following tables 1 to 42 and the tables 43 to 840, wherein the combination of variables B¹, B², B³, and B⁴ for each compound corresponds to each line of Table B. Each of the groups mentioned for a substituent in the tables is furthermore per se, independently of the combination in which it is mentioned, a particularly preferred aspect of the substituent in question.

Table 1. Compounds of formula 1-1 where R^{1A} is H, R^{1B} is H, R^{1C} is H, R^{1D} is H, Ar¹ is Ar¹-2,
Table 2. Compounds of formula I-1 where R^{1A} is H, R^{1B} is H, R¹⁰ is H, R^{1D} is H, Ar¹ is Ar¹-3.
Table 3. Compounds of formula 1-1 where R^{1A} is H, R^{1B} is H, R¹⁰ is H, R^{1D} is H, Ar¹ is Ar¹-4.
Table 4. Compounds of formula 1-1 where R^{1A} is H, R^{1B} is H, R^{1C} is H, R^{1D} is H, Ar¹ is Ar¹-5.
Table 5. Compounds of formula 1-1 where R^{1A} is H, R^{1B} is H, R¹⁰ is H, R^{1D} is H, Ar¹ is Ar¹-6.
Table 6. Compounds of formula 1-1 where R^{1A} is H, R^{1B} is H, R^{1C} is H, R^{1D} is H, Ar¹ is Ar¹-7.
Table 7. Compounds of formula 1-1 where R^{1A} is H, R^{1B} is H, R^{1C} is H, R^{1D} is H, Ar¹ is Ar¹-8.
Table 8. Compounds of formula 1-1 where R^{1A} is H, R^{1B} is OCF₃, R^{1C} is H, R^{1D} is H, Ar¹ is Ar¹-2.
Table 9. Compounds of formula 1-1 where R^{1A} is H, R^{1B} is OCF₃, R^{1C} is H, R^{1D} is H, Ar¹ is Ar¹-3.
Table 10. Compounds of formula 1-1 where R^{1A} is H, R^{1B} is OCF₃, R^{1C} is H, R^{1D} is H, Ar¹ is Ar¹-4.
Table 11. Compounds of formula 1-1 where R^{1A} is H, R^{1B} is OCF₃, R^{1C} is H, R^{1D} is H, Ar¹ is Ar¹-5.
Table 12. Compounds of formula 1-1 where R^{1A} is H, R^{1B} is OCF₃, R^{1C} is H, R^{1D} is H, Ar¹ is Ar¹-6.
Table 13. Compounds of formula 1-1 where R^{1A} is H, R^{1B} is OCF₃, R^{1C} is H, R^{1D} is H, Ar¹ is Ar¹-7.
Table 14. Compounds of formula 1-1 where R^{1A} is H, R^{1B} is OCF₃, R^{1C} is H, R^{1D} is H, Ar¹ is Ar¹-8.
Table 15. Compounds of formula 1-1 where R^{1A} is H, R^{1B} is CF₃, R^{1C} is H, R^{1D} is H, Ar¹ is Ar¹-2.
Table 16. Compounds of formula 1-1 where R^{1A} is H, R^{1B} is CF₃, R^{1C} is H, R^{1D} is H, Ar¹ is Ar¹-3.
Table 17. Compounds of formula 1-1 where R^{1A} is H, R^{1B} is CF₃, R^{1C} is H, R^{1D} is H, Ar¹ is Ar¹-4.
Table 18. Compounds of formula I-1 where R^{1A} is H, R^{1B} is CF₃, R^{1C} is H, R^{1D} is H, Ar¹ is Ar¹-5.
Table 19. Compounds of formula 1-1 where R^{1A} is H, R^{1B} is CF₃, R^{1C} is H, R^{1D} is H, Ar¹ is Ar¹-6.
Table 20. Compounds of formula 1-1 where R^{1A} is H, R^{1B} is CF₃, R^{1C} is H, R^{1D} is H, Ar¹ is Ar¹-7.
Table 21. Compounds of formula 1-1 where R^{1A} is H, R^{1B} is CF₃, R^{1C} is H, R^{1D} is H, Ar¹ is Ar¹-8.
Table 22. Compounds of formula 1-1 where R^{1A} is H, R^{1B} is Cl, R^{1C} is H, R^{1D} is H, Ar¹ is Ar¹-2.
Table 23. Compounds of formula 1-1 where R^{1A} is H, R^{1B} is Cl, R^{1C} is H, R^{1D} is H, Ar¹ is Ar¹-3.
Table 24. Compounds of formula 1-1 where R^{1A} is H, R^{1B} is Cl, R^{1C} is H, R^{1D} is H, Ar¹ is Ar¹-4.
Table 25. Compounds of formula 1-1 where R^{1A} is H, R^{1B} is Cl, R^{1C} is H, R^{1D} is H, Ar¹ is Ar¹-5.
Table 26. Compounds of formula 1-1 where R^{1A} is H, R^{1B} is Cl, R^{1C} is H, R^{1D} is H, Ar¹ is Ar¹-6.
Table 27. Compounds of formula 1-1 where R^{1A} is H, R^{1B} is Cl, R^{1C} is H, R^{1D} is H, Ar¹ is Ar¹-7.
Table 28. Compounds of formula 1-1 where R^{1A} is H, R^{1B} is Cl, R^{1C} is H, R^{1D} is H, Ar¹ is Ar¹-8.
Table 29. Compounds of formula I-1 where R^{1A} is H, R^{1B} is F, R^{1C} is H, R^{1D} is H, Ar¹ is Ar¹-2.
Table 30. Compounds of formula 1-1 where R^{1A} is H, R^{1B} is F, R^{1C} is H, R^{1D} is H, Ar¹ is Ar¹-3.
Table 31. Compounds of formula 1-1 where R^{1A} is H, R^{1B} is F, R^{1C} is H, R^{1D} is H, Ar¹ is Ar¹-4.
Table 32. Compounds of formula 1-1 where R^{1A} is H, R^{1B} is F, R^{1C} is H, R^{1D} is H, Ar¹ is Ar¹-5.
Table 33. Compounds of formula I-1 where R^{1A} is H, R^{1B} is F, R^{1C} is H, R^{1D} is H, Ar¹ is Ar¹-6.
Table 34. Compounds of formula 1-1 where R^{1A} is H, R^{1B} is F, R^{1C} is H, R^{1D} is H, Ar¹ is Ar¹-7.
Table 35. Compounds of formula 1-1 where R^{1A} is H, R^{1B} is F, R^{1C} is H, R^{1D} is H, Ar¹ is Ar¹-8.
Table 36. Compounds of formula 1-1 where R^{1A} is H, R^{1B} is CN, R^{1C} is H, R^{1D} is H, Ar¹ is Ar¹-2.
Table 37. Compounds of formula I-1 where R^{1A} is H, R^{1B} is CN, R^{1C} is H, R^{1D} is H, Ar¹ is Ar¹-3.
Table 38. Compounds of formula 1-1 where R^{1A} is H, R^{1B} is CN, R^{1C} is H, R^{1D} is H, Ar¹ is Ar¹-4.
Table 39. Compounds of formula 1-1 where R^{1A} is H, R^{1B} is CN, R^{1C} is H, R^{1D} is H, Ar¹ is Ar¹-5.
Table 40. Compounds of formula 1-1 where R^{1A} is H, R^{1B} is CN, R^{1C} is H, R^{1D} is H, Ar¹ is Ar¹-6.
Table 41. Compounds of formula I-1 where R^{1A} is H, R^{1B} is CN, R^{1C} is H, R^{1D} is H, Ar¹ is Ar¹-7.
Table 42. Compounds of formula 1-1 where R^{1A} is H, R^{1B} is CN, R^{1C} is H, R^{1D} is H, Ar¹ is Ar¹-8.
Table 43. Compounds of formula 1-1 where R^{1A} is H, R^{1B} is OCF₃, R¹⁰ is H, R^{1D} is H, Ar¹ is Ar¹-1.
Table 44. Compounds of formula 1-1 where R^{1A} is H, R^{1B} is OCF₃, R^{1C} is H, R^{1D} is H, Ar¹ is Ar¹-9.
Table 45. Compounds of formula 1-1 where R^{1A} is H, R^{1B} is OCF₃, R¹⁰ is H, R^{1D} is H, Ar¹ is Ar¹-15.
Table 46. Compounds of formula 1-1 where R^{1A} is H, R^{1B} is CF₃, R^{1C} is H, R^{1D} is H, Ar¹ is Ar¹-1.
Table 47. Compounds of formula 1-1 where R^{1A} is H, R^{1B} is CF₃, R¹⁰ is H, R^{1D} is H, Ar¹ is Ar¹-9.
Table 48. Compounds of formula 1-1 where R^{1A} is H, R^{1B} is CF₃, R^{1C} is H, R^{1D} is H, Ar¹ is Ar¹-15.
Table 49. Compounds of formula I-1 where R^{1A} is H, R^{1B} is Cl, R¹⁰ is H, R^{1D} is H, Ar¹ is Ar¹-1.
Table 50. Compounds of formula 1-1 where R^{1A} is H, R^{1B} is Cl, R^{1C} is H, R^{1D} is H, Ar¹ is Ar¹-9.
Table 51. Compounds of formula I-1 where R^{1A} is H, R^{1B} is Cl, R^{1C} is H, R^{1D} is H, Ar¹ is Ar¹-15.
Table 52. Compounds of formula 1-1 where R^{1A} is H, R^{1B} is F, R^{1C} is H, R^{1D} is H, Ar¹ is Ar¹-1.
Table 53. Compounds of formula 1-1 where R^{1A} is H, R^{1B} is F, R^{1C} is H, R^{1D} is H, Ar¹ is Ar¹-9.
Table 54. Compounds of formula 1-1 where R^{1A} is H, R^{1B} is F, R^{1C} is H, R^{1D} is H, Ar¹ is Ar¹-15.
Table 55. Compounds of formula 1-1 where R^{1A} is H, R^{1B} is CN, R^{1C} is H, R^{1D} is H, Ar¹ is Ar¹-1.
Table 56. Compounds of formula I-1 where R^{1A} is H, R^{1B} is CN, R^{1C} is H, R^{1D} is H, Ar¹ is Ar¹-9.
Table 57. Compounds of formula 1-1 where R^{1A} is H, R^{1B} is CN, R^{1C} is H, R^{1D} is H, Ar¹ is Ar¹-15.
Table 58. Compounds of formula 1-1 where R^{1A} is H, R^{1B} is Cl, R^{1C} is H, R^{1D} is F, Ar¹ is Ar¹-1.
Table 59. Compounds of formula I-1 where R^{1A} is H, R^{1B} is Cl, R^{1C} is H, R^{1D} is F, Ar¹ is Ar¹-2.
Table 60. Compounds of formula I-1 where R^{1A} is H, R^{1B} is Cl, R^{1C} is H, R^{1D} is F, Ar¹ is Ar¹-3.
Table 61. Compounds of formula 1-1 where R^{1A} is H, R^{1B} is Cl, R^{1C} is H, R^{1D} is F, Ar¹ is Ar¹-4.
Table 62. Compounds of formula 1-1 where R^{1A} is H, R^{1B} is Cl, R^{1C} is H, R^{1D} is F, Ar¹ is Ar¹-5.
Table 63. Compounds of formula 1-1 where R^{1A} is H, R^{1B} is Cl, R^{1C} is H, R^{1D} is F, Ar¹ is Ar¹-6.
Table 64. Compounds of formula 1-1 where R^{1A} is H, R^{1B} is Cl, R^{1C} is H, R^{1D} is F, Ar¹ is Ar¹-7.
Table 65. Compounds of formula 1-1 where R^{1A} is H, R^{1B} is Cl, R¹⁰ is H, R^{1D} is F, Ar¹ is Ar¹-8.
Table 66. Compounds of formula 1-1 where R^{1A} is H, R^{1B} is Cl, R^{1C} is H, R^{1D} is F, Ar¹ is Ar¹-9.
Table 67. Compounds of formula I-1 where R^{1A} is H, R^{1B} is Cl, R¹⁰ is H, R^{1D} is F, Ar¹ is Ar¹-15.

TABLE B

| Line | B¹ | B² | B³ | B⁴ |
|---|---|---|---|---|
| 1. | N | N | CH | CH |
| 2. | N | N | CH | C—Cl |
| 3. | N | N | CH | C—F |
| 4. | N | N | CH | C—Br |
| 5. | N | N | CH | C—CH₃ |
| 6. | N | N | C—Cl | CH |
| 7. | N | N | C—Cl | C—Cl |
| 8. | N | N | C—Cl | C—F |
| 9. | N | N | C—Cl | C—Br |
| 10. | N | N | C—Cl | C—CH₃ |
| 11. | N | N | C—F | CH |
| 12. | N | N | C—F | C—Cl |
| 13. | N | N | C—F | C—F |
| 14. | N | N | C—F | C—Br |
| 15. | N | N | C—F | C—CH₃ |
| 16. | N | N | C—Br | CH |
| 17. | N | N | C—Br | C—Cl |
| 18. | N | N | C—Br | C—F |
| 19. | N | N | C—Br | C—Br |
| 20. | N | N | C—Br | C—CH₃ |
| 21. | N | N | C—CH₃ | CH |
| 22. | N | N | C—CH₃ | C—Cl |
| 23. | N | N | C—CH₃ | C—F |
| 24. | N | N | C—CH₃ | C—Br |
| 25. | N | N | C—CH₃ | C—CH₃ |
| 26. | N | CH | N | CH |
| 27. | N | CH | N | C—Cl |
| 28. | N | CH | N | C—F |
| 29. | N | CH | N | C—Br |
| 30. | N | CH | N | C—CH₃ |
| 31. | N | CH | CH | CH |
| 32. | N | CH | CH | C—Cl |
| 33. | N | CH | CH | C—F |
| 34. | N | CH | CH | C—Br |
| 35. | N | CH | CH | C—CH₃ |
| 36. | N | CH | C—Cl | CH |
| 37. | N | CH | C—Cl | C—Cl |
| 38. | N | CH | C—Cl | C—F |
| 39. | N | CH | C—Cl | C—Br |
| 40. | N | CH | C—Cl | C—CH₃ |
| 41. | N | CH | C—F | CH |
| 42. | N | CH | C—F | C—Cl |
| 43. | N | CH | C—F | C—F |
| 44. | N | CH | C—F | C—Br |
| 45. | N | CH | C—F | C—CH₃ |
| 46. | N | CH | C—Br | CH |
| 47. | N | CH | C—Br | C—Cl |
| 48. | N | CH | C—Br | C—F |
| 49. | N | CH | C—Br | C—Br |
| 50. | N | CH | C—Br | C—CH₃ |
| 51. | N | CH | C—CH₃ | CH |
| 52. | N | CH | C—CH₃ | C—Cl |
| 53. | N | CH | C—CH₃ | C—F |
| 54. | N | CH | C—CH₃ | C—Br |
| 55. | N | CH | C—CH₃ | C—CH₃ |
| 56. | N | C—Cl | N | CH |
| 57. | N | C—Cl | N | C—Cl |
| 58. | N | C—Cl | N | C—F |
| 59. | N | C—Cl | N | C—Br |
| 60. | N | C—Cl | N | C—CH₃ |
| 61. | N | C—Cl | CH | CH |
| 62. | N | C—Cl | CH | C—Cl |
| 63. | N | C—Cl | CH | C—F |
| 64. | N | C—Cl | CH | C—Br |
| 65. | N | C—Cl | CH | C—CH₃ |
| 66. | N | C—Cl | C—Cl | CH |
| 67. | N | C—Cl | C—Cl | C—Cl |
| 68. | N | C—Cl | C—Cl | C—F |
| 69. | N | C—Cl | C—Cl | C—Br |
| 70. | N | C—Cl | C—Cl | C—CH₃ |
| 71. | N | C—Cl | C—F | CH |
| 72. | N | C—Cl | C—F | C—Cl |
| 73. | N | C—Cl | C—F | C—F |
| 74. | N | C—Cl | C—F | C—Br |
| 75. | N | C—Cl | C—F | C—CH₃ |
| 76. | N | C—Cl | C—Br | CH |
| 77. | N | C—Cl | C—Br | C—Cl |
| 78. | N | C—Cl | C—Br | C—F |
| 79. | N | C—Cl | C—Br | C—Br |
| 80. | N | C—Cl | C—Br | C—CH₃ |
| 81. | N | C—Cl | C—CH₃ | CH |
| 82. | N | C—Cl | C—CH₃ | C—Cl |
| 83. | N | C—Cl | C—CH₃ | C—F |
| 84. | N | C—Cl | C—CH₃ | C—Br |
| 85. | N | C—Cl | C—CH₃ | C—CH₃ |
| 86. | N | C—F | N | CH |
| 87. | N | C—F | N | C—Cl |
| 88. | N | C—F | N | C—F |
| 89. | N | C—F | N | C—Br |
| 90. | N | C—F | N | C—CH₃ |
| 91. | N | C—F | CH | CH |
| 92. | N | C—F | CH | C—Cl |
| 93. | N | C—F | CH | C—F |
| 94. | N | C—F | CH | C—Br |
| 95. | N | C—F | CH | C—CH₃ |
| 96. | N | C—F | C—Cl | CH |
| 97. | N | C—F | C—Cl | C—Cl |
| 98. | N | C—F | C—Cl | C—F |
| 99. | N | C—F | C—Cl | C—Br |
| 100. | N | C—F | C—Cl | C—CH₃ |
| 101. | N | C—F | C—F | CH |
| 102. | N | C—F | C—F | C—Cl |
| 103. | N | C—F | C—F | C—F |
| 104. | N | C—F | C—F | C—Br |
| 105. | N | C—F | C—F | C—CH₃ |
| 106. | N | C—F | C—Br | CH |
| 107. | N | C—F | C—Br | C—Cl |
| 108. | N | C—F | C—Br | C—F |
| 109. | N | C—F | C—Br | C—Br |
| 110. | N | C—F | C—Br | C—CH₃ |
| 111. | N | C—F | C—CH₃ | CH |
| 112. | N | C—F | C—CH₃ | C—Cl |
| 113. | N | C—F | C—CH₃ | C—F |
| 114. | N | C—F | C—CH₃ | C—Br |
| 115. | N | C—F | C—CH₃ | C—CH₃ |
| 116. | N | C—Br | N | CH |
| 117. | N | C—Br | N | C—Cl |
| 118. | N | C—Br | N | C—F |
| 119. | N | C—Br | N | C—Br |
| 120. | N | C—Br | N | C—CH₃ |
| 121. | N | C—Br | CH | CH |
| 122. | N | C—Br | CH | C—Cl |
| 123. | N | C—Br | CH | C—F |
| 124. | N | C—Br | CH | C—Br |
| 125. | N | C—Br | CH | C—CH₃ |
| 126. | N | C—Br | C—Cl | CH |
| 127. | N | C—Br | C—Cl | C—Cl |
| 128. | N | C—Br | C—Cl | C—F |
| 129. | N | C—Br | C—Cl | C—Br |
| 130. | N | C—Br | C—Cl | C—CH₃ |
| 131. | N | C—Br | C—F | CH |
| 132. | N | C—Br | C—F | C—Cl |
| 133. | N | C—Br | C—F | C—F |
| 134. | N | C—Br | C—F | C—Br |
| 135. | N | C—Br | C—F | C—CH₃ |
| 136. | N | C—Br | C—Br | CH |
| 137. | N | C—Br | C—Br | C—Cl |
| 138. | N | C—Br | C—Br | C—F |
| 139. | N | C—Br | C—Br | C—Br |
| 140. | N | C—Br | C—Br | C—CH₃ |
| 141. | N | C—Br | C—CH₃ | CH |
| 142. | N | C—Br | C—CH₃ | C—Cl |
| 143. | N | C—Br | C—CH₃ | C—F |
| 144. | N | C—Br | C—CH₃ | C—Br |
| 145. | N | C—Br | C—CH₃ | C—CH₃ |
| 146. | N | C—CH₃ | N | CH |
| 147. | N | C—CH₃ | N | C—Cl |
| 148. | N | C—CH₃ | N | C—F |
| 149. | N | C—CH₃ | N | C—Br |
| 150. | N | C—CH₃ | N | C—CH₃ |
| 151. | N | C—CH₃ | CH | CH |
| 152. | N | C—CH₃ | CH | C—Cl |
| 153. | N | C—CH₃ | CH | C—F |
| 154. | N | C—CH₃ | CH | C—Br |
| 155. | N | C—CH₃ | CH | C—CH₃ |
| 156. | N | C—CH₃ | C—Cl | CH |
| 157. | N | C—CH₃ | C—Cl | C—Cl |
| 158 | N | C—CH₃ | C—Cl | C—F |
| 159. | N | C—CH₃ | C—Cl | C—Br |
| 160. | N | C—CH₃ | C—Cl | C—CH₃ |
| 161. | N | C—CH₃ | C—F | CH |
| 162. | N | C—CH₃ | C—F | C—Cl |
| 163. | N | C—CH₃ | C—F | C—F |
| 164. | N | C—CH₃ | C—F | C—Br |
| 165. | N | C—CH₃ | C—F | C—CH₃ |
| 166. | N | C—CH₃ | C—Br | CH |
| 167. | N | C—CH₃ | C—Br | C—Cl |
| 168. | N | C—CH₃ | C—Br | C—F |
| 169. | N | C—CH₃ | C—Br | C—Br |
| 170. | N | C—CH₃ | C—Br | C—CH₃ |
| 171. | N | C—CH₃ | C—CH₃ | CH |
| 172 | N | C—CH₃ | C—CH₃ | C—Cl |
| 173. | N | C—CH₃ | C—CH₃ | C—F |
| 174. | N | C—CH₃ | C—CH₃ | C—Br |
| 175. | N | C—CH₃ | C—CH₃ | C—CH₃ |
| 176 | CH | N | N | CH |
| 177. | CH | N | N | C—Cl |
| 178. | CH | N | N | C—F |
| 179. | CH | N | N | C—Br |
| 180. | CH | N | N | C—CH₃ |
| 181. | CH | N | CH | CH |
| 182. | CH | N | CH | C—Cl |
| 183 | CH | N | CH | C—F |
| 184. | CH | N | CH | C—Br |
| 185. | CH | N | CH | C—CH₃ |
| 186. | CH | N | C—Cl | CH |
| 187. | CH | N | C—Cl | C—Cl |
| 188. | CH | N | C—Cl | C—F |
| 189. | CH | N | C—Cl | C—Br |
| 190. | CH | N | C—Cl | C—CH₃ |
| 191. | CH | N | C—F | CH |
| 192. | CH | N | C—F | C—Cl |
| 193. | CH | N | C—F | C—F |
| 194. | CH | N | C—F | C—Br |
| 195. | CH | N | C—F | C—CH₃ |
| 196. | CH | N | C—Br | CH |
| 197. | CH | N | C—Br | C—Cl |
| 198. | CH | N | C—Br | C—F |
| 199. | CH | N | C—Br | C—Br |
| 200. | CH | N | C—Br | C—CH₃ |
| 201. | CH | N | C—CH₃ | CH |
| 202. | CH | N | C—CH₃ | C—Cl |
| 203. | CH | N | C—CH₃ | C—F |
| 204. | CH | N | C—CH₃ | C—Br |
| 205. | CH | N | C—CH₃ | C—CH₃ |
| 206. | CH | CH | N | CH |
| 207. | CH | CH | N | C—Cl |
| 208. | CH | CH | N | C—F |
| 209. | CH | CH | N | C—Br |
| 210. | CH | CH | N | C—CH₃ |
| 211. | CH | CH | CH | CH |
| 212. | CH | CH | CH | C—Cl |
| 213. | CH | CH | CH | C—F |
| 214. | CH | CH | CH | C—Br |
| 215. | CH | CH | CH | C—CH₃ |
| 216. | CH | CH | C—Cl | CH |
| 217. | CH | CH | C—Cl | C—Cl |
| 218. | CH | CH | C—Cl | C—F |
| 219. | CH | CH | C—Cl | C—Br |
| 220. | CH | CH | C—Cl | C—CH₃ |
| 221. | CH | CH | C—F | CH |
| 222. | CH | CH | C—F | C—Cl |
| 223. | CH | CH | C—F | C—F |
| 224. | CH | CH | C—F | C—Br |
| 225. | CH | CH | C—F | C—CH₃ |
| 226. | CH | CH | C—Br | CH |
| 227. | CH | CH | C—Br | C—Cl |
| 228. | CH | CH | C—Br | C—F |
| 229. | CH | CH | C—Br | C—Br |
| 230. | CH | CH | C—Br | C—CH₃ |
| 231. | CH | CH | C—CH₃ | CH |
| 232. | CH | CH | C—CH₃ | C—Cl |
| 233. | CH | CH | C—CH₃ | C—F |
| 234. | CH | CH | C—CH₃ | C—Br |
| 235. | CH | CH | C—CH₃ | C—CH₃ |
| 236. | CH | C—Cl | N | CH |
| 237. | CH | C—Cl | N | C—Cl |
| 238. | CH | C—Cl | N | C—F |
| 239. | CH | C—Cl | N | C—Br |
| 240. | CH | C—Cl | N | C—CH₃ |
| 241. | CH | C—Cl | CH | CH |
| 242. | CH | C—Cl | CH | C—Cl |
| 243. | CH | C—Cl | CH | C—F |
| 244. | CH | C—Cl | CH | C—Br |
| 245. | CH | C—Cl | CH | C—CH₃ |
| 246. | CH | C—Cl | C—Cl | CH |
| 247. | CH | C—Cl | C—Cl | C—Cl |
| 248. | CH | C—Cl | C—Cl | C—F |
| 249. | CH | C—Cl | C—Cl | C—Br |
| 250. | CH | C—Cl | C—Cl | C—CH₃ |
| 251. | CH | C—Cl | C—F | CH |
| 252. | CH | C—Cl | C—F | C—Cl |
| 253. | CH | C—Cl | C—F | C—F |
| 254. | CH | C—Cl | C—F | C—Br |
| 255. | CH | C—Cl | C—F | C—CH₃ |
| 256. | CH | C—Cl | C—Br | CH |
| 257. | CH | C—Cl | C—Br | C—Cl |
| 258. | CH | C—Cl | C—Br | C—F |
| 259. | CH | C—Cl | C—Br | C—Br |
| 260. | CH | C—Cl | C—Br | C—CH₃ |
| 261. | CH | C—Cl | C—CH₃ | CH |
| 262. | CH | C—Cl | C—CH₃ | C—Cl |
| 263. | CH | C—Cl | C—CH₃ | C—F |
| 264. | CH | C—Cl | C—CH₃ | C—Br |
| 265. | CH | C—Cl | C—CH₃ | C—CH₃ |
| 266. | CH | C—F | N | CH |
| 267. | CH | C—F | N | C—Cl |
| 268. | CH | C—F | N | C—F |
| 269. | CH | C—F | N | C—Br |
| 270. | CH | C—F | N | C—CH₃ |
| 271. | CH | C—F | CH | CH |
| 272. | CH | C—F | CH | C—Cl |
| 273. | CH | C—F | CH | C—F |
| 274. | CH | C—F | CH | C—Br |
| 275. | CH | C—F | CH | C—CH₃ |
| 276. | CH | C—F | C—Cl | CH |
| 277. | CH | C—F | C—Cl | C—Cl |
| 278. | CH | C—F | C—Cl | C—F |
| 279. | CH | C—F | C—Cl | C—Br |
| 280. | CH | C—F | C—Cl | C—CH₃ |
| 281. | CH | C—F | C—F | CH |
| 282. | CH | C—F | C—F | C—Cl |
| 283. | CH | C—F | C—F | C—F |
| 284. | CH | C—F | C—F | C—Br |
| 285. | CH | C—F | C—F | C—CH₃ |
| 286. | CH | C—F | C—Br | CH |
| 287. | CH | C—F | C—Br | C—Cl |
| 288. | CH | C—F | C—Br | C—F |
| 289. | CH | C—F | C—Br | C—Br |
| 290. | CH | C—F | C—Br | C—CH₃ |
| 291. | CH | C—F | C—CH₃ | CH |
| 292. | CH | C—F | C—CH₃ | C—Cl |
| 293. | CH | C—F | C—CH₃ | C—F |
| 294. | CH | C—F | C—CH₃ | C—Br |
| 295. | CH | C—F | C—CH₃ | C—CH₃ |
| 296. | CH | C—Br | N | CH |
| 297. | CH | C—Br | N | C—Cl |
| 298. | CH | C—Br | N | C—F |
| 299. | CH | C—Br | N | C—Br |
| 300. | CH | C—Br | N | C—CH₃ |
| 301. | CH | C—Br | CH | CH |
| 302. | CH | C—Br | CH | C—Cl |
| 303. | CH | C—Br | CH | C—F |
| 304. | CH | C—Br | CH | C—Br |
| 305. | CH | C—Br | CH | C—CH₃ |
| 306. | CH | C—Br | C—Cl | CH |
| 307. | CH | C—Br | C—Cl | C—Cl |
| 308. | CH | C—Br | C—Cl | C—F |
| 309. | CH | C—Br | C—Cl | C—Br |
| 310. | CH | C—Br | C—Cl | C—CH₃ |
| 311. | CH | C—Br | C—F | CH |
| 312. | CH | C—Br | C—F | C—Cl |
| 313. | CH | C—Br | C—F | C—F |
| 314. | CH | C—Br | C—F | C—Br |
| 315. | CH | C—Br | C—F | C—CH₃ |
| 316. | CH | C—Br | C—Br | CH |
| 317. | CH | C—Br | C—Br | C—Cl |
| 318. | CH | C—Br | C—Br | C—F |
| 319. | CH | C—Br | C—Br | C—Br |
| 320. | CH | C—Br | C—Br | C—CH₃ |
| 321. | CH | C—Br | C—CH₃ | CH |
| 322. | CH | C—Br | C—CH₃ | C—Cl |
| 323. | CH | C—Br | C—CH₃ | C—F |
| 324. | CH | C—Br | C—CH₃ | C—Br |
| 325. | CH | C—Br | C—CH₃ | C—CH₃ |
| 326. | CH | C—CH₃ | N | CH |
| 327. | CH | C—CH₃ | N | C—Cl |
| 328. | CH | C—CH₃ | N | C—F |
| 329. | CH | C—CH₃ | N | C—Br |
| 330. | CH | C—CH₃ | N | C—CH₃ |
| 331. | CH | C—CH₃ | CH | CH |
| 332. | CH | C—CH₃ | CH | C—Cl |
| 333. | CH | C—CH₃ | CH | C—F |
| 334. | CH | C—CH₃ | CH | C—Br |
| 335. | CH | C—CH₃ | CH | C—CH₃ |
| 336. | CH | C—CH₃ | C—Cl | CH |
| 337. | CH | C—CH₃ | C—Cl | C—Cl |
| 338. | CH | C—CH₃ | C—Cl | C—F |
| 339. | CH | C—CH₃ | C—Cl | C—Br |
| 340. | CH | C—CH₃ | C—Cl | C—CH₃ |
| 341. | CH | C—CH₃ | C—F | CH |
| 342. | CH | C—CH₃ | C—F | C—Cl |
| 343. | CH | C—CH₃ | C—F | C—F |
| 344. | CH | C—CH₃ | C—F | C—Br |
| 345. | CH | C—CH₃ | C—F | C—CH₃ |
| 346. | CH | C—CH₃ | C—Br | CH |
| 347. | CH | C—CH₃ | C—Br | C—Cl |
| 348. | CH | C—CH₃ | C—Br | C—F |
| 349. | CH | C—CH₃ | C—Br | C—Br |
| 350. | CH | C—CH₃ | C—Br | C—CH₃ |
| 351. | CH | C—CH₃ | C—CH₃ | CH |
| 352. | CH | C—CH₃ | C—CH₃ | C—Cl |
| 353. | CH | C—CH₃ | C—CH₃ | C—F |
| 354. | CH | C—CH₃ | C—CH₃ | C—Br |
| 355. | CH | C—CH₃ | C—CH₃ | C—CH₃ |
| 356. | C—Cl | N | N | CH |
| 357. | C—Cl | N | N | C—Cl |
| 358. | C—Cl | N | N | C—F |
| 359. | C—Cl | N | N | C—Br |
| 360. | C—Cl | N | N | C—CH₃ |
| 361. | C—Cl | N | CH | CH |
| 362. | C—Cl | N | CH | C—Cl |
| 363. | C—Cl | N | CH | C—F |
| 364. | C—Cl | N | CH | C—Br |
| 365. | C—Cl | N | CH | C—CH₃ |
| 366. | C—Cl | N | C—Cl | CH |
| 367. | C—Cl | N | C—Cl | C—Cl |
| 368. | C—Cl | N | C—Cl | C—F |
| 369. | C—Cl | N | C—Cl | C—Br |
| 370. | C—Cl | N | C—Cl | C—CH₃ |
| 371. | C—Cl | N | C—F | CH |
| 372. | C—Cl | N | C—F | C—Cl |
| 373. | C—Cl | N | C—F | C—F |
| 374. | C—Cl | N | C—F | C—Br |
| 375. | C—Cl | N | C—F | C—CH₃ |
| 376. | C—Cl | N | C—Br | CH |
| 377. | C—Cl | N | C—Br | C—Cl |
| 378. | C—Cl | N | C—Br | C—F |
| 379. | C—Cl | N | C—Br | C—Br |
| 380. | C—Cl | N | C—Br | C—CH₃ |
| 381. | C—Cl | N | C—CH₃ | CH |
| 382. | C—Cl | N | C—CH₃ | C—Cl |
| 383. | C—Cl | N | C—CH₃ | C—F |
| 384. | C—Cl | N | C—CH₃ | C—Br |
| 385. | C—Cl | N | C—CH₃ | C—CH₃ |
| 386. | C—Cl | CH | N | CH |
| 387. | C—Cl | CH | N | C—Cl |
| 388. | C—Cl | CH | N | C—F |
| 389. | C—Cl | CH | N | C—Br |
| 390. | C—Cl | CH | N | C—CH₃ |
| 391. | C—Cl | CH | CH | CH |
| 392. | C—Cl | CH | CH | C—Cl |
| 393. | C—Cl | CH | CH | C—F |
| 394. | C—Cl | CH | CH | C—Br |
| 395. | C—Cl | CH | CH | C—CH₃ |
| 396. | C—Cl | CH | C—Cl | CH |
| 397. | C—Cl | CH | C—Cl | C—Cl |
| 398. | C—Cl | CH | C—Cl | C—F |
| 399. | C—Cl | CH | C—Cl | C—Br |
| 400. | C—Cl | CH | C—Cl | C—CH₃ |
| 401. | C—Cl | CH | C—F | CH |
| 402. | C—Cl | CH | C—F | C—Cl |
| 403. | C—Cl | CH | C—F | C—F |
| 404. | C—Cl | CH | C—F | C—Br |
| 405. | C—Cl | CH | C—F | C—CH₃ |
| 406. | C—Cl | CH | C—Br | CH |
| 407. | C—Cl | CH | C—Br | C—Cl |
| 408. | C—Cl | CH | C—Br | C—F |
| 409. | C—Cl | CH | C—Br | C—Br |
| 410. | C—Cl | CH | C—Br | C—CH₃ |
| 411. | C—Cl | CH | C—CH₃ | CH |
| 412. | C—Cl | CH | C—CH₃ | C—Cl |
| 413. | C—Cl | CH | C—CH₃ | C—F |
| 414. | C—Cl | CH | C—CH₃ | C—Br |
| 415. | C—Cl | CH | C—CH₃ | C—CH₃ |
| 416. | C—Cl | C—Cl | N | CH |
| 417. | C—Cl | C—Cl | N | C—Cl |
| 418. | C—Cl | C—Cl | N | C—F |
| 419. | C—Cl | C—Cl | N | C—Br |
| 420. | C—Cl | C—Cl | N | C—CH₃ |
| 421. | C—Cl | C—Cl | CH | CH |
| 422. | C—Cl | C—Cl | CH | C—Cl |
| 423. | C—Cl | C—Cl | CH | C—F |
| 424. | C—Cl | C—Cl | CH | C—Br |
| 425. | C—Cl | C—Cl | CH | C—CH₃ |
| 426. | C—Cl | C—Cl | C—Cl | CH |
| 427. | C—Cl | C—Cl | C—Cl | C—Cl |
| 428. | C—Cl | C—Cl | C—Cl | C—F |
| 429. | C—Cl | C—Cl | C—Cl | C—Br |
| 430. | C—Cl | C—Cl | C—Cl | C—CH₃ |
| 431. | C—Cl | C—Cl | C—F | CH |
| 432. | C—Cl | C—Cl | C—F | C—Cl |
| 433. | C—Cl | C—Cl | C—F | C—F |
| 434. | C—Cl | C—Cl | C—F | C—Br |
| 435. | C—Cl | C—Cl | C—F | C—CH₃ |
| 436. | C—Cl | C—Cl | C—Br | CH |
| 437. | C—Cl | C—Cl | C—Br | C—Cl |
| 438. | C—Cl | C—Cl | C—Br | C—F |
| 439. | C—Cl | C—Cl | C—Br | C—Br |
| 440. | C—Cl | C—Cl | C—Br | C—CH₃ |
| 441. | C—Cl | C—Cl | C—CH₃ | CH |
| 442. | C—Cl | C—Cl | C—CH₃ | C—Cl |
| 443. | C—Cl | C—Cl | C—CH₃ | C—F |
| 444. | C—Cl | C—Cl | C—CH₃ | C—Br |
| 445. | C—Cl | C—Cl | C—CH₃ | C—CH₃ |
| 446. | C—Cl | C—F | N | CH |
| 447. | C—Cl | C—F | N | C—Cl |
| 448. | C—Cl | C—F | N | C—F |
| 449. | C—Cl | C—F | N | C—Br |
| 450. | C—Cl | C—F | N | C—CH₃ |
| 451. | C—Cl | C—F | CH | CH |
| 452. | C—Cl | C—F | CH | C—Cl |
| 453. | C—Cl | C—F | CH | C—F |
| 454. | C—Cl | C—F | CH | C—Br |
| 455. | C—Cl | C—F | CH | C—CH₃ |
| 456. | C—Cl | C—F | C—Cl | CH |
| 457. | C—Cl | C—F | C—Cl | C—Cl |
| 458. | C—Cl | C—F | C—Cl | C—F |
| 459. | C—Cl | C—F | C—Cl | C—Br |
| 460. | C—Cl | C—F | C—Cl | C—CH₃ |
| 461. | C—Cl | C—F | C—F | CH |
| 462. | C—Cl | C—F | C—F | C—Cl |
| 463. | C—Cl | C—F | C—F | C—F |
| 464. | C—Cl | C—F | C—F | C—Br |
| 465. | C—Cl | C—F | C—F | C—CH₃ |
| 466. | C—Cl | C—F | C—Br | CH |
| 467. | C—Cl | C—F | C—Br | C—Cl |
| 468. | C—Cl | C—F | C—Br | C—F |
| 469. | C—Cl | C—F | C—Br | C—Br |
| 470. | C—Cl | C—F | C—Br | C—CH₃ |
| 471. | C—Cl | C—F | C—CH₃ | CH |
| 472. | C—Cl | C—F | C—CH₃ | C—Cl |
| 473. | C—Cl | C—F | C—CH₃ | C—F |
| 474. | C—Cl | C—F | C—CH₃ | C—Br |
| 475. | C—Cl | C—F | C—CH₃ | C—CH₃ |
| 476. | C—Cl | C—Br | N | CH |
| 477. | C—Cl | C—Br | N | C—Cl |
| 478. | C—Cl | C—Br | N | C—F |
| 479. | C—Cl | C—Br | N | C—Br |
| 480. | C—Cl | C—Br | N | C—CH₃ |
| 481. | C—Cl | C—Br | CH | CH |
| 482. | C—Cl | C—Br | CH | C—Cl |
| 483. | C—Cl | C—Br | CH | C—F |
| 484. | C—Cl | C—Br | CH | C—Br |
| 485. | C—Cl | C—Br | CH | C—CH₃ |
| 486. | C—Cl | C—Br | C—Cl | CH |
| 487. | C—Cl | C—Br | C—Cl | C—Cl |
| 488. | C—Cl | C—Br | C—Cl | C—F |
| 489. | C—Cl | C—Br | C—Cl | C—Br |
| 490. | C—Cl | C—Br | C—Cl | C—CH₃ |
| 491. | C—Cl | C—Br | C—F | CH |
| 492. | C—Cl | C—Br | C—F | C—Cl |
| 493. | C—Cl | C—Br | C—F | C—F |
| 494. | C—Cl | C—Br | C—F | C—Br |
| 495. | C—Cl | C—Br | C—F | C—CH₃ |
| 496. | C—Cl | C—Br | C—Br | CH |
| 497. | C—Cl | C—Br | C—Br | C—Cl |
| 498. | C—Cl | C—Br | C—Br | C—F |
| 499. | C—Cl | C—Br | C—Br | C—Br |
| 500. | C—Cl | C—Br | C—Br | C—CH₃ |
| 501. | C—Cl | C—Br | C—CH₃ | CH |
| 502. | C—Cl | C—Br | C—CH₃ | C—Cl |
| 503. | C—Cl | C—Br | C—CH₃ | C—F |
| 504. | C—Cl | C—Br | C—CH₃ | C—Br |
| 505. | C—Cl | C—Br | C—CH₃ | C—CH₃ |
| 506. | C—Cl | C—CH₃ | N | CH |
| 507. | C—Cl | C—CH₃ | N | C—Cl |
| 508. | C—Cl | C—CH₃ | N | C—F |
| 509. | C—Cl | C—CH₃ | N | C—Br |
| 510. | C—Cl | C—CH₃ | N | C—CH₃ |
| 511. | C—Cl | C—CH₃ | CH | CH |
| 512. | C—Cl | C—CH₃ | CH | C—Cl |
| 513. | C—Cl | C—CH₃ | CH | C—F |
| 514. | C—Cl | C—CH₃ | CH | C—Br |
| 515. | C—Cl | C—CH₃ | CH | C—CH₃ |
| 516. | C—Cl | C—CH₃ | C—Cl | CH |
| 517. | C—Cl | C—CH₃ | C—Cl | C—Cl |
| 518. | C—Cl | C—CH₃ | C—Cl | C—F |
| 519. | C—Cl | C—CH₃ | C—Cl | C—Br |
| 520. | C—Cl | C—CH₃ | C—Cl | C—CH₃ |
| 521. | C—Cl | C—CH₃ | C—F | CH |
| 522. | C—Cl | C—CH₃ | C—F | C—Cl |
| 523. | C—Cl | C—CH₃ | C—F | C—F |
| 524. | C—Cl | C—CH₃ | C—F | C—Br |
| 525. | C—Cl | C—CH₃ | C—F | C—CH₃ |
| 526. | C—Cl | C—CH₃ | C—Br | CH |
| 527. | C—Cl | C—CH₃ | C—Br | C—Cl |
| 528. | C—Cl | C—CH₃ | C—Br | C—F |
| 529. | C—Cl | C—CH₃ | C—Br | C—Br |
| 530. | C—Cl | C—CH₃ | C—Br | C—CH₃ |
| 531. | C—Cl | C—CH₃ | C—CH₃ | CH |
| 532. | C—Cl | C—CH₃ | C—CH₃ | C—Cl |
| 533. | C—Cl | C—CH₃ | C—CH₃ | C—F |
| 534. | C—Cl | C—CH₃ | C—CH₃ | C—Br |
| 535. | C—Cl | C—CH₃ | C—CH₃ | C—CH₃ |
| 536. | C—F | N | N | CH |
| 537. | C—F | N | N | C—Cl |
| 538. | C—F | N | N | C—F |
| 539. | C—F | N | N | C—Br |
| 540. | C—F | N | N | C—CH₃ |
| 541. | C—F | N | CH | CH |
| 542. | C—F | N | CH | C—Cl |
| 543. | C—F | N | CH | C—F |
| 544. | C—F | N | CH | C—Br |
| 545. | C—F | N | CH | C—CH₃ |
| 546. | C—F | N | C—Cl | CH |
| 547. | C—F | N | C—Cl | C—Cl |
| 548. | C—F | N | C—Cl | C—F |
| 549. | C—F | N | C—Cl | C—Br |
| 550. | C—F | N | C—Cl | C—CH₃ |
| 551. | C—F | N | C—F | CH |
| 552. | C—F | N | C—F | C—Cl |
| 553. | C—F | N | C—F | C—F |
| 554. | C—F | N | C—F | C—Br |
| 555. | C—F | N | C—F | C—CH₃ |
| 556. | C—F | N | C—Br | CH |
| 557. | C—F | N | C—Br | C—Cl |
| 558. | C—F | N | C—Br | C—F |
| 559 | C—F | N | C—Br | C—Br |
| 560. | C—F | N | C—Br | C—CH₃ |
| 561. | C—F | N | C—CH₃ | CH |
| 562. | C—F | N | C—CH₃ | C—Cl |
| 563. | C—F | N | C—CH₃ | C—F |
| 564. | C—F | N | C—CH₃ | C—Br |
| 565. | C—F | N | C—CH₃ | C—CH₃ |
| 566. | C—F | CH | N | CH |
| 567. | C—F | CH | N | C—Cl |
| 568. | C—F | CH | N | C—F |
| 569. | C—F | CH | N | C—Br |
| 570. | C—F | CH | N | C—CH₃ |
| 571. | C—F | CH | CH | CH |
| 572. | C—F | CH | CH | C—Cl |
| 573. | C—F | CH | CH | C—F |
| 574. | C—F | CH | CH | C—Br |
| 575. | C—F | CH | CH | C—CH₃ |
| 576. | C—F | CH | C—Cl | CH |
| 577. | C—F | CH | C—Cl | C—Cl |
| 578. | C—F | CH | C—Cl | C—F |
| 579. | C—F | CH | C—Cl | C—Br |
| 580. | C—F | CH | C—Cl | C—CH₃ |
| 581. | C—F | CH | C—F | CH |
| 582. | C—F | CH | C—F | C—Cl |
| 583. | C—F | CH | C—F | C—F |
| 584. | C—F | CH | C—F | C—Br |
| 585. | C—F | CH | C—F | C—CH₃ |
| 586. | C—F | CH | C—Br | CH |
| 587. | C—F | CH | C—Br | C—Cl |
| 588. | C—F | CH | C—Br | C—F |
| 589. | C—F | CH | C—Br | C—Br |
| 590. | C—F | CH | C—Br | C—CH₃ |
| 591. | C—F | CH | C—CH₃ | CH |
| 592. | C—F | CH | C—CH₃ | C—Cl |
| 593. | C—F | CH | C—CH₃ | C—F |
| 594. | C—F | CH | C—CH₃ | C—Br |
| 595. | C—F | CH | C—CH₃ | C—CH₃ |
| 596. | C—F | C—Cl | N | CH |
| 597. | C—F | C—Cl | N | C—Cl |
| 598. | C—F | C—Cl | N | C—F |
| 599. | C—F | C—Cl | N | C—Br |
| 600. | C—F | C—Cl | N | C—CH₃ |
| 601. | C—F | C—Cl | CH | CH |
| 602. | C—F | C—Cl | CH | C—Cl |
| 603. | C—F | C—Cl | CH | C—F |
| 604. | C—F | C—Cl | CH | C—Br |
| 605. | C—F | C—Cl | CH | C—CH₃ |
| 606. | C—F | C—Cl | C—Cl | CH |
| 607. | C—F | C—Cl | C—Cl | C—Cl |
| 608. | C—F | C—Cl | C—Cl | C—F |
| 609. | C—F | C—Cl | C—Cl | C—Br |
| 610. | C—F | C—Cl | C—Cl | C—CH₃ |
| 611. | C—F | C—Cl | C—F | CH |
| 612. | C—F | C—Cl | C—F | C—Cl |
| 613. | C—F | C—Cl | C—F | C—F |
| 614. | C—F | C—Cl | C—F | C—Br |
| 615. | C—F | C—Cl | C—F | C—CH₃ |
| 616. | C—F | C—Cl | C—Br | CH |
| 617. | C—F | C—Cl | C—Br | C—Cl |
| 618. | C—F | C—Cl | C—Br | C—F |
| 619. | C—F | C—Cl | C—Br | C—Br |
| 620. | C—F | C—Cl | C—Br | C—CH₃ |
| 621. | C—F | C—Cl | C—CH₃ | CH |
| 622. | C—F | C—Cl | C—CH₃ | C—Cl |
| 623. | C—F | C—Cl | C—CH₃ | C—F |
| 624. | C—F | C—Cl | C—CH₃ | C—Br |
| 625. | C—F | C—Cl | C—CH₃ | C—CH₃ |
| 626. | C—F | C—F | N | CH |
| 627. | C—F | C—F | N | C—Cl |
| 628. | C—F | C—F | N | C—F |
| 629. | C—F | C—F | N | C—Br |
| 630. | C—F | C—F | N | C—CH₃ |
| 631. | C—F | C—F | CH | CH |
| 632. | C—F | C—F | CH | C—Cl |
| 633. | C—F | C—F | CH | C—F |
| 634. | C—F | C—F | CH | C—Br |
| 635. | C—F | C—F | CH | C—CH₃ |
| 636. | C—F | C—F | C—Cl | CH |
| 637. | C—F | C—F | C—Cl | C—Cl |
| 638. | C—F | C—F | C—Cl | C—F |
| 639. | C—F | C—F | C—Cl | C—Br |
| 640. | C—F | C—F | C—Cl | C—CH₃ |
| 641. | C—F | C—F | C—F | CH |
| 642. | C—F | C—F | C—F | C—Cl |
| 643. | C—F | C—F | C—F | C—F |
| 644. | C—F | C—F | C—F | C—Br |
| 645. | C—F | C—F | C—F | C—CH₃ |
| 646. | C—F | C—F | C—Br | CH |
| 647. | C—F | C—F | C—Br | C—Cl |
| 648. | C—F | C—F | C—Br | C—F |
| 649. | C—F | C—F | C—Br | C—Br |
| 650. | C—F | C—F | C—Br | C—CH₃ |
| 651. | C—F | C—F | C—CH₃ | CH |
| 652. | C—F | C—F | C—CH₃ | C—Cl |
| 653. | C—F | C—F | C—CH₃ | C—F |
| 654. | C—F | C—F | C—CH₃ | C—Br |
| 655. | C—F | C—F | C—CH₃ | C—CH₃ |
| 656. | C—F | C—Br | N | CH |
| 657. | C—F | C—Br | N | C—Cl |
| 658. | C—F | C—Br | N | C—F |
| 659. | C—F | C—Br | N | C—Br |
| 660. | C—F | C—Br | N | C—CH₃ |
| 661. | C—F | C—Br | CH | CH |
| 662. | C—F | C—Br | CH | C—Cl |
| 663. | C—F | C—Br | CH | C—F |
| 664. | C—F | C—Br | CH | C—Br |
| 665. | C—F | C—Br | CH | C—CH₃ |
| 666. | C—F | C—Br | C—Cl | CH |
| 667. | C—F | C—Br | C—Cl | C—Cl |
| 668. | C—F | C—Br | C—Cl | C—F |
| 669. | C—F | C—Br | C—Cl | C—Br |
| 670. | C—F | C—Br | C—Cl | C—CH₃ |
| 671. | C—F | C—Br | C—F | CH |
| 672. | C—F | C—Br | C—F | C—Cl |
| 673. | C—F | C—Br | C—F | C—F |
| 674. | C—F | C—Br | C—F | C—Br |
| 675. | C—F | C—Br | C—F | C—CH₃ |
| 676. | C—F | C—Br | C—Br | CH |
| 677. | C—F | C—Br | C—Br | C—Cl |
| 678. | C—F | C—Br | C—Br | C—F |
| 679. | C—F | C—Br | C—Br | C—Br |
| 680. | C—F | C—Br | C—Br | C—CH₃ |
| 681. | C—F | C—Br | C—CH₃ | CH |
| 682. | C—F | C—Br | C—CH₃ | C—Cl |
| 683. | C—F | C—Br | C—CH₃ | C—F |
| 684. | C—F | C—Br | C—CH₃ | C—Br |
| 685. | C—F | C—Br | C—CH₃ | C—CH₃ |
| 686. | C—F | C—CH₃ | N | CH |
| 687. | C—F | C—CH₃ | N | C—Cl |
| 688. | C—F | C—CH₃ | N | C—F |
| 689. | C—F | C—CH₃ | N | C—Br |
| 690. | C—F | C—CH₃ | N | C—CH₃ |
| 691. | C—F | C—CH₃ | CH | CH |
| 692. | C—F | C—CH₃ | CH | C—Cl |
| 693. | C—F | C—CH₃ | CH | C—F |
| 694. | C—F | C—CH₃ | CH | C—Br |
| 695. | C—F | C—CH₃ | CH | C—CH₃ |
| 696. | C—F | C—CH₃ | C—Cl | CH |
| 697. | C—F | C—CH₃ | C—Cl | C—Cl |
| 698. | C—F | C—CH₃ | C—Cl | C—F |
| 699. | C—F | C—CH₃ | C—Cl | C—Br |
| 700. | C—F | C—CH₃ | C—Cl | C—CH₃ |
| 701. | C—F | C—CH₃ | C—F | CH |
| 702. | C—F | C—CH₃ | C—F | C—Cl |
| 703. | C—F | C—CH₃ | C—F | C—F |
| 704. | C—F | C—CH₃ | C—F | C—Br |
| 705. | C—F | C—CH₃ | C—F | C—CH₃ |
| 706. | C—F | C—CH₃ | C—Br | CH |
| 707. | C—F | C—CH₃ | C—Br | C—Cl |
| 708. | C—F | C—CH₃ | C—Br | C—F |
| 709. | C—F | C—CH₃ | C—Br | C—Br |
| 710. | C—F | C—CH₃ | C—Br | C—CH₃ |
| 711. | C—F | C—CH₃ | C—CH₃ | CH |
| 712. | C—F | C—CH₃ | C—CH₃ | C—Cl |
| 713. | C—F | C—CH₃ | C—CH₃ | C—F |
| 714. | C—F | C—CH₃ | C—CH₃ | C—Br |
| 715. | C—F | C—CH₃ | C—CH₃ | C—CH₃ |
| 716. | C—Br | N | N | CH |
| 717. | C—Br | N | N | C—Cl |
| 718. | C—Br | N | N | C—F |
| 719. | C—Br | N | N | C—Br |
| 720. | C—Br | N | N | C—CH₃ |
| 721. | C—Br | N | CH | CH |
| 722. | C—Br | N | CH | C—Cl |
| 723. | C—Br | N | CH | C—F |
| 724. | C—Br | N | CH | C—Br |
| 725. | C—Br | N | CH | C—CH₃ |
| 726. | C—Br | N | C—Cl | CH |
| 727. | C—Br | N | C—Cl | C—Cl |
| 728. | C—Br | N | C—Cl | C—F |
| 729. | C—Br | N | C—Cl | C—Br |
| 730. | C—Br | N | C—Cl | C—CH₃ |
| 731. | C—Br | N | C—F | CH |
| 732. | C—Br | N | C—F | C—Cl |
| 733. | C—Br | N | C—F | C—F |
| 734. | C—Br | N | C—F | C—Br |
| 735. | C—Br | N | C—F | C—CH₃ |
| 736. | C—Br | N | C—Br | CH |
| 737. | C—Br | N | C—Br | C—Cl |
| 738. | C—Br | N | C—Br | C—F |
| 739. | C—Br | N | C—Br | C—Br |
| 740. | C—Br | N | C—Br | C—CH₃ |
| 741. | C—Br | N | C—CH₃ | CH |
| 742. | C—Br | N | C—CH₃ | C—Cl |
| 743. | C—Br | N | C—CH₃ | C—F |
| 744. | C—Br | N | C—CH₃ | C—Br |
| 745. | C—Br | N | C—CH₃ | C—CH₃ |
| 746. | C—Br | CH | N | CH |
| 747. | C—Br | CH | N | C—Cl |
| 748. | C—Br | CH | N | C—F |
| 749. | C—Br | CH | N | C—Br |
| 750. | C—Br | CH | N | C—CH₃ |
| 751. | C—Br | CH | CH | CH |
| 752. | C—Br | CH | CH | C—Cl |
| 753. | C—Br | CH | CH | C—F |
| 754. | C—Br | CH | CH | C—Br |
| 755. | C—Br | CH | CH | C—CH₃ |
| 756. | C—Br | CH | C—Cl | CH |
| 757. | C—Br | CH | C—Cl | C—Cl |
| 758. | C—Br | CH | C—Cl | C—F |
| 759. | C—Br | CH | C—Cl | C—Br |
| 760. | C—Br | CH | C—Cl | C—CH₃ |
| 761. | C—Br | CH | C—F | CH |
| 762. | C—Br | CH | C—F | C—Cl |
| 763. | C—Br | CH | C—F | C—F |
| 764. | C—Br | CH | C—F | C—Br |
| 765. | C—Br | CH | C—F | C—CH₃ |
| 766. | C—Br | CH | C—Br | CH |
| 767. | C—Br | CH | C—Br | C—Cl |
| 768. | C—Br | CH | C—Br | C—F |
| 769. | C—Br | CH | C—Br | C—Br |
| 770. | C—Br | CH | C—Br | C—CH₃ |
| 771. | C—Br | CH | C—CH₃ | CH |
| 772. | C—Br | CH | C—CH₃ | C—Cl |
| 773. | C—Br | CH | C—CH₃ | C—F |
| 774. | C—Br | CH | C—CH₃ | C—Br |
| 775. | C—Br | CH | C—CH₃ | C—CH₃ |
| 776. | C—Br | C—Cl | N | CH |
| 777. | C—Br | C—Cl | N | C—Cl |
| 778. | C—Br | C—Cl | N | C—F |
| 779. | C—Br | C—Cl | N | C—Br |
| 780. | C—Br | C—Cl | N | C—CH₃ |
| 781. | C—Br | C—Cl | CH | CH |
| 782. | C—Br | C—Cl | CH | C—Cl |
| 783. | C—Br | C—Cl | CH | C—F |
| 784 | C—Br | C—Cl | CH | C—Br |
| 785. | C—Br | C—Cl | CH | C—CH₃ |
| 786. | C—Br | C—Cl | C—Cl | CH |
| 787. | C—Br | C—Cl | C—Cl | C—Cl |
| 788. | C—Br | C—Cl | C—Cl | C—F |
| 789. | C—Br | C—Cl | C—Cl | C—Br |
| 790. | C—Br | C—Cl | C—Cl | C—CH₃ |
| 791. | C—Br | C—Cl | C—F | CH |
| 792. | C—Br | C—Cl | C—F | C—Cl |
| 793. | C—Br | C—Cl | C—F | C—F |
| 794. | C—Br | C—Cl | C—F | C—Br |
| 795. | C—Br | C—Cl | C—F | C—CH₃ |
| 796. | C—Br | C—Cl | C—Br | CH |
| 797. | C—Br | C—Cl | C—Br | C—Cl |
| 798. | C—Br | C—Cl | C—Br | C—F |
| 799. | C—Br | C—Cl | C—Br | C—Br |
| 800. | C—Br | C—Cl | C—Br | C—CH₃ |
| 801. | C—Br | C—Cl | C—CH₃ | CH |
| 802. | C—Br | C—Cl | C—CH₃ | C—Cl |
| 803. | C—Br | C—Cl | C—CH₃ | C—F |
| 804. | C—Br | C—Cl | C—CH₃ | C—Br |
| 805. | C—Br | C—Cl | C—CH₃ | C—CH₃ |
| 806. | C—Br | C—F | N | CH |
| 807. | C—Br | C—F | N | C—Cl |
| 808. | C—Br | C—F | N | C—F |
| 809. | C—Br | C—F | N | C—Br |
| 810. | C—Br | C—F | N | C—CH₃ |
| 811. | C—Br | C—F | CH | CH |
| 812. | C—Br | C—F | CH | C—Cl |
| 813. | C—Br | C—F | CH | C—F |
| 814. | C—Br | C—F | CH | C—Br |
| 815. | C—Br | C—F | CH | C—CH₃ |
| 816. | C—Br | C—F | C—Cl | CH |
| 817. | C—Br | C—F | C—Cl | C—Cl |
| 818. | C—Br | C—F | C—Cl | C—F |
| 819. | C—Br | C—F | C—Cl | C—Br |
| 820. | C—Br | C—F | C—Cl | C—CH₃ |
| 821. | C—Br | C—F | C—F | CH |
| 822. | C—Br | C—F | C—F | C—Cl |
| 823. | C—Br | C—F | C—F | C—F |
| 824. | C—Br | C—F | C—F | C—Br |
| 825. | C—Br | C—F | C—F | C—CH₃ |
| 826. | C—Br | C—F | C—Br | CH |
| 827. | C—Br | C—F | C—Br | C—Cl |
| 828. | C—Br | C—F | C—Br | C—F |
| 829. | C—Br | C—F | C—Br | C—Br |
| 830. | C—Br | C—F | C—Br | C—CH₃ |
| 831. | C—Br | C—F | C—CH₃ | CH |
| 832. | C—Br | C—F | C—CH₃ | C—Cl |
| 833. | C—Br | C—F | C—CH₃ | C—F |
| 834. | C—Br | C—F | C—CH₃ | C—Br |
| 835. | C—Br | C—F | C—CH₃ | C—CH₃ |
| 836. | C—Br | C—Br | N | CH |
| 837. | C—Br | C—Br | N | C—Cl |
| 838. | C—Br | C—Br | N | C—F |
| 839. | C—Br | C—Br | N | C—Br |
| 840. | C—Br | C—Br | N | C—CH₃ |
| 841. | C—Br | C—Br | CH | CH |
| 842. | C—Br | C—Br | CH | C—Cl |
| 843. | C—Br | C—Br | CH | C—F |
| 844. | C—Br | C—Br | CH | C—Br |
| 845. | C—Br | C—Br | CH | C—CH₃ |
| 846. | C—Br | C—Br | C—Cl | CH |
| 847. | C—Br | C—Br | C—Cl | C—Cl |
| 848. | C—Br | C—Br | C—Cl | C—F |
| 849. | C—Br | C—Br | C—Cl | C—Br |
| 850. | C—Br | C—Br | C—Cl | C—CH₃ |
| 851. | C—Br | C—Br | C—F | CH |
| 852. | C—Br | C—Br | C—F | C—Cl |
| 853. | C—Br | C—Br | C—F | C—F |
| 854. | C—Br | C—Br | C—F | C—Br |
| 855. | C—Br | C—Br | C—F | C—CH₃ |
| 856. | C—Br | C—Br | C—Br | CH |
| 857. | C—Br | C—Br | C—Br | C—Cl |
| 858. | C—Br | C—Br | C—Br | C—F |
| 859. | C—Br | C—Br | C—Br | C—Br |
| 860. | C—Br | C—Br | C—Br | C—CH₃ |
| 861. | C—Br | C—Br | C—CH₃ | CH |
| 862. | C—Br | C—Br | C—CH₃ | C—Cl |
| 863. | C—Br | C—Br | C—CH₃ | C—F |
| 864. | C—Br | C—Br | C—CH₃ | C—Br |
| 865. | C—Br | C—Br | C—CH₃ | C—CH₃ |
| 866. | C—Br | C—CH₃ | N | CH |
| 867. | C—Br | C—CH₃ | N | C—Cl |
| 868. | C—Br | C—CH₃ | N | C—F |
| 869. | C—Br | C—CH₃ | N | C—Br |
| 870. | C—Br | C—CH₃ | N | C—CH₃ |
| 871. | C—Br | C—CH₃ | CH | CH |
| 872. | C—Br | C—CH₃ | CH | C—Cl |
| 873. | C—Br | C—CH₃ | CH | C—F |
| 874. | C—Br | C—CH₃ | CH | C—Br |
| 875. | C—Br | C—CH₃ | CH | C—CH₃ |
| 876. | C—Br | C—CH₃ | C—Cl | CH |
| 877. | C—Br | C—CH₃ | C—Cl | C—Cl |
| 878. | C—Br | C—CH₃ | C—Cl | C—F |
| 879. | C—Br | C—CH₃ | C—Cl | C—Br |
| 880. | C—Br | C—CH₃ | C—Cl | C—CH₃ |
| 881. | C—Br | C—CH₃ | C—F | CH |
| 882. | C—Br | C—CH₃ | C—F | C—Cl |
| 883. | C—Br | C—CH₃ | C—F | C—F |
| 884. | C—Br | C—CH₃ | C—F | C—Br |
| 885. | C—Br | C—CH₃ | C—F | C—CH₃ |
| 886. | C—Br | C—CH₃ | C—Br | CH |
| 887. | C—Br | C—CH₃ | C—Br | C—Cl |
| 888. | C—Br | C—CH₃ | C—Br | C—F |
| 889. | C—Br | C—CH₃ | C—Br | C—Br |
| 890. | C—Br | C—CH₃ | C—Br | C—CH₃ |
| 891. | C—Br | C—CH₃ | C—CH₃ | CH |
| 892. | C—Br | C—CH₃ | C—CH₃ | C—Cl |
| 893. | C—Br | C—CH₃ | C—CH₃ | C—F |
| 894. | C—Br | C—CH₃ | C—CH₃ | C—Br |
| 895. | C—Br | C—CH₃ | C—CH₃ | C—CH₃ |
| 896. | C—CH₃ | N | N | CH |
| 897. | C—CH₃ | N | N | C—Cl |
| 898. | C—CH₃ | N | N | C—F |
| 899. | C—CH₃ | N | N | C—Br |
| 900. | C—CH₃ | N | N | C—CH₃ |
| 901. | C—CH₃ | N | CH | CH |
| 902. | C—CH₃ | N | CH | C—Cl |
| 903. | C—CH₃ | N | CH | C—F |
| 904. | C—CH₃ | N | CH | C—Br |
| 905. | C—CH₃ | N | CH | C—CH₃ |
| 906. | C—CH₃ | N | C—Cl | CH |
| 907. | C—CH₃ | N | C—Cl | C—Cl |
| 908. | C—CH₃ | N | C—Cl | C—F |
| 909. | C—CH₃ | N | C—Cl | C—Br |
| 910. | C—CH₃ | N | C—Cl | C—CH₃ |
| 911. | C—CH₃ | N | C—F | CH |
| 912. | C—CH₃ | N | C—F | C—Cl |
| 913. | C—CH₃ | N | C—F | C—F |
| 914. | C—CH₃ | N | C—F | C—Br |
| 915. | C—CH₃ | N | C—F | C—CH₃ |
| 916. | C—CH₃ | N | C—Br | CH |
| 917. | C—CH₃ | N | C—Br | C—Cl |
| 918. | C—CH₃ | N | C—Br | C—F |
| 919. | C—CH₃ | N | C—Br | C—Br |
| 920. | C—CH₃ | N | C—Br | C—CH₃ |
| 921. | C—CH₃ | N | C—CH₃ | CH |
| 922. | C—CH₃ | N | C—CH₃ | C—Cl |
| 923. | C—CH₃ | N | C—CH₃ | C—F |
| 924. | C—CH₃ | N | C—CH₃ | C—Br |
| 925. | C—CH₃ | N | C—CH₃ | C—CH₃ |
| 926. | C—CH₃ | CH | N | CH |
| 927. | C—CH₃ | CH | N | C—Cl |
| 928. | C—CH₃ | CH | N | C—F |
| 929. | C—CH₃ | CH | N | C—Br |
| 930. | C—CH₃ | CH | N | C—CH₃ |
| 931. | C—CH₃ | CH | CH | CH |
| 932. | C—CH₃ | CH | CH | C—Cl |
| 933. | C—CH₃ | CH | CH | C—F |
| 934. | C—CH₃ | CH | CH | C—Br |
| 935. | C—CH₃ | CH | CH | C—CH₃ |
| 936. | C—CH₃ | CH | C—Cl | CH |
| 937. | C—CH₃ | CH | C—Cl | C—Cl |
| 938. | C—CH₃ | CH | C—Cl | C—F |
| 939. | C—CH₃ | CH | C—Cl | C—Br |
| 940. | C—CH₃ | CH | C—Cl | C—CH₃ |
| 941. | C—CH₃ | CH | C—F | CH |
| 942. | C—CH₃ | CH | C—F | C—Cl |
| 943. | C—CH₃ | CH | C—F | C—F |
| 944. | C—CH₃ | CH | C—F | C—Br |
| 945. | C—CH₃ | CH | C—F | C—CH₃ |
| 946. | C—CH₃ | CH | C—Br | CH |
| 947. | C—CH₃ | CH | C—Br | C—Cl |
| 948. | C—CH₃ | CH | C—Br | C—F |
| 949. | C—CH₃ | CH | C—Br | C—Br |
| 950. | C—CH₃ | CH | C—Br | C—CH₃ |
| 951. | C—CH₃ | CH | C—CH₃ | CH |
| 952. | C—CH₃ | CH | C—CH₃ | C—Cl |
| 953. | C—CH₃ | CH | C—CH₃ | C—F |
| 954. | C—CH₃ | CH | C—CH₃ | C—Br |
| 955. | C—CH₃ | CH | C—CH₃ | C—CH₃ |
| 956. | C—CH₃ | C—Cl | N | CH |
| 957. | C—CH₃ | C—Cl | N | C—Cl |
| 958. | C—CH₃ | C—Cl | N | C—F |
| 959. | C—CH₃ | C—Cl | N | C—Br |
| 960. | C—CH₃ | C—Cl | N | C—CH₃ |
| 961. | C—CH₃ | C—Cl | CH | CH |
| 962. | C—CH₃ | C—Cl | CH | C—Cl |
| 963. | C—CH₃ | C—Cl | CH | C—F |
| 964. | C—CH₃ | C—Cl | CH | C—Br |
| 965. | C—CH₃ | C—Cl | CH | C—CH₃ |
| 966. | C—CH₃ | C—Cl | C—Cl | CH |
| 967. | C—CH₃ | C—Cl | C—Cl | C—Cl |
| 968. | C—CH₃ | C—Cl | C—Cl | C—F |
| 969. | C—CH₃ | C—Cl | C—Cl | C—Br |
| 970. | C—CH₃ | C—Cl | C—Cl | C—CH₃ |
| 971. | C—CH₃ | C—Cl | C—F | CH |
| 972. | C—CH₃ | C—Cl | C—F | C—Cl |
| 973. | C—CH₃ | C—Cl | C—F | C—F |
| 974. | C—CH₃ | C—Cl | C—F | C—Br |
| 975. | C—CH₃ | C—Cl | C—F | C—CH₃ |
| 976. | C—CH₃ | C—Cl | C—Br | CH |
| 977. | C—CH₃ | C—Cl | C—Br | C—Cl |
| 978. | C—CH₃ | C—Cl | C—Br | C—F |
| 979. | C—CH₃ | C—Cl | C—Br | C—Br |
| 980. | C—CH₃ | C—Cl | C—Br | C—CH₃ |
| 981. | C—CH₃ | C—Cl | C—CH₃ | CH |
| 982. | C—CH₃ | C—Cl | C—CH₃ | C—Cl |
| 983. | C—CH₃ | C—Cl | C—CH₃ | C—F |
| 984. | C—CH₃ | C—Cl | C—CH₃ | C—Br |
| 985. | C—CH₃ | C—Cl | C—CH₃ | C—CH₃ |
| 986. | C—CH₃ | C—F | N | CH |
| 987. | C—CH₃ | C—F | N | C—Cl |
| 988. | C—CH₃ | C—F | N | C—F |
| 989. | C—CH₃ | C—F | N | C—Br |
| 990. | C—CH₃ | C—F | N | C—CH₃ |
| 991. | C—CH₃ | C—F | CH | CH |
| 992. | C—CH₃ | C—F | CH | C—Cl |
| 993. | C—CH₃ | C—F | CH | C—F |
| 994. | C—CH₃ | C—F | CH | C—Br |
| 995. | C—CH₃ | C—F | CH | C—CH₃ |
| 996. | C—CH₃ | C—F | C—Cl | CH |
| 997. | C—CH₃ | C—F | C—Cl | C—Cl |
| 998. | C—CH₃ | C—F | C—Cl | C—F |
| 999. | C—CH₃ | C—F | C—Cl | C—Br |
| 1000. | C—CH₃ | C—F | C—Cl | C—CH₃ |
| 1001. | C—CH₃ | C—F | C—F | CH |
| 1002. | C—CH₃ | C—F | C—F | C—Cl |
| 1003. | C—CH₃ | C—F | C—F | C—F |
| 1004. | C—CH₃ | C—F | C—F | C—Br |
| 1005. | C—CH₃ | C—F | C—F | C—CH₃ |
| 1006. | C—CH₃ | C—F | C—Br | CH |
| 1007. | C—CH₃ | C—F | C—Br | C—Cl |
| 1008. | C—CH₃ | C—F | C—Br | C—F |
| 1009. | C—CH₃ | C—F | C—Br | C—Br |
| 1010. | C—CH₃ | C—F | C—Br | C—CH₃ |
| 1011. | C—CH₃ | C—F | C—CH₃ | CH |
| 1012. | C—CH₃ | C—F | C—CH₃ | C—Cl |
| 1013. | C—CH₃ | C—F | C—CH₃ | C—F |
| 1014. | C—CH₃ | C—F | C—CH₃ | C—Br |
| 1015. | C—CH₃ | C—F | C—CH₃ | C—CH₃ |
| 1016. | C—CH₃ | C—Br | N | CH |
| 1017. | C—CH₃ | C—Br | N | C—Cl |
| 1018. | C—CH₃ | C—Br | N | C—F |
| 1019. | C—CH₃ | C—Br | N | C—Br |
| 1020. | C—CH₃ | C—Br | N | C—CH₃ |
| 1021. | C—CH₃ | C—Br | CH | CH |
| 1022. | C—CH₃ | C—Br | CH | C—Cl |
| 1023. | C—CH₃ | C—Br | CH | C—F |
| 1024. | C—CH₃ | C—Br | CH | C—Br |
| 1025. | C—CH₃ | C—Br | CH | C—CH₃ |
| 1026. | C—CH₃ | C—Br | C—Cl | CH |
| 1027. | C—CH₃ | C—Br | C—Cl | C—Cl |
| 1028. | C—CH₃ | C—Br | C—Cl | C—F |
| 1029. | C—CH₃ | C—Br | C—Cl | C—Br |
| 1030. | C—CH₃ | C—Br | C—Cl | C—CH₃ |
| 1031. | C—CH₃ | C—Br | C—F | CH |
| 1032. | C—CH₃ | C—Br | C—F | C—Cl |
| 1033. | C—CH₃ | C—Br | C—F | C—F |
| 1034. | C—CH₃ | C—Br | C—F | C—Br |
| 1035. | C—CH₃ | C—Br | C—F | C—CH₃ |
| 1036. | C—CH₃ | C—Br | C—Br | CH |
| 1037. | C—CH₃ | C—Br | C—Br | C—Cl |
| 1038. | C—CH₃ | C—Br | C—Br | C—F |
| 1039. | C—CH₃ | C—Br | C—Br | C—Br |
| 1040. | C—CH₃ | C—Br | C—Br | C—CH₃ |
| 1041. | C—CH₃ | C—Br | C—CH₃ | CH |
| 1042. | C—CH₃ | C—Br | C—CH₃ | C—Cl |
| 1043. | C—CH₃ | C—Br | C—CH₃ | C—F |
| 1044. | C—CH₃ | C—Br | C—CH₃ | C—Br |
| 1045. | C—CH₃ | C—Br | C—CH₃ | C—CH₃ |
| 1046. | C—CH₃ | C—CH₃ | N | CH |
| 1047. | C—CH₃ | C—CH₃ | N | C—Cl |
| 1048. | C—CH₃ | C—CH₃ | N | C—F |
| 1049. | C—CH₃ | C—CH₃ | N | C—Br |
| 1050. | C—CH₃ | C—CH₃ | N | C—CH₃ |
| 1051. | C—CH₃ | C—CH₃ | CH | CH |
| 1052. | C—CH₃ | C—CH₃ | CH | C—Cl |
| 1053. | C—CH₃ | C—CH₃ | CH | C—F |
| 1054. | C—CH₃ | C—CH₃ | CH | C—Br |
| 1055. | C—CH₃ | C—CH₃ | CH | C—CH₃ |
| 1056. | C—CH₃ | C—CH₃ | C—Cl | CH |
| 1057. | C—CH₃ | C—CH₃ | C—Cl | C—Cl |
| 1058. | C—CH₃ | C—CH₃ | C—Cl | C—F |
| 1059. | C—CH₃ | C—CH₃ | C—Cl | C—Br |
| 1060. | C—CH₃ | C—CH₃ | C—Cl | C—CH₃ |
| 1061. | C—CH₃ | C—CH₃ | C—F | CH |
| 1062. | C—CH₃ | C—CH₃ | C—F | C—Cl |
| 1063. | C—CH₃ | C—CH₃ | C—F | C—F |
| 1064. | C—CH₃ | C—CH₃ | C—F | C—Br |
| 1065. | C—CH₃ | C—CH₃ | C—F | C—CH₃ |
| 1066. | C—CH₃ | C—CH₃ | C—Br | CH |
| 1067. | C—CH₃ | C—CH₃ | C—Br | C—Cl |
| 1068. | C—CH₃ | C—CH₃ | C—Br | C—F |
| 1069. | C—CH₃ | C—CH₃ | C—Br | C—Br |
| 1070. | C—CH₃ | C—CH₃ | C—Br | C—CH₃ |
| 1071. | C—CH₃ | C—CH₃ | C—CH₃ | CH |
| 1072. | C—CH₃ | C—CH₃ | C—CH₃ | C—Cl |
| 1073. | C—CH₃ | C—CH₃ | C—CH₃ | C—F |
| 1074. | C—CH₃ | C—CH₃ | C—CH₃ | C—Br |
| 1075. | C—CH₃ | C—CH₃ | C—CH₃ | C—CH₃ |

In various preferred embodiments, B¹-B⁴ are as defined in lines 31, 211, 212, 213, 241, 271, 301, or 331 in the above tables.
Table 43 to Table 84: Includes all the compounds as disclosed in Table 1 to Table 42 respectively wherein compound of formula 1-1 is replaced by compound of formula 1-2;
Table 85 to Table 126: Includes all the compounds as disclosed in Table 1 to Table 42 respectively wherein compound of formula 1-1 is replaced by compound of formula 1-3;
Table 127 to Table 168: Includes all the compounds as disclosed in Table 1 to Table 42 respectively wherein compound of formula 1-1 is replaced by compound of formula 1-4;
Table 169 to Table 210: Includes all the compounds as disclosed in Table 1 to Table 42 respectively wherein compound of formula 1-1 is replaced by compound of formula 1-5;
Table 211 to Table 252: Includes all the compounds as disclosed in Table 1 to Table 42 respectively wherein compound of formula 1-1 is replaced by compound of formula 1-6;
Table 253 to Table 294: Includes all the compounds as disclosed in Table 1 to Table 42 respectively wherein compound of formula 1-1 is replaced by compound of formula 1-7;
Table 295 to Table 336: Includes all the compounds as disclosed in Table 1 to Table 42 respectively wherein compound of formula 1-1 is replaced by compound of formula 1-8;
Table 337 to Table 378: Includes all the compounds as disclosed in Table 1 to Table 42 respectively wherein compound of formula 1-1 is replaced by compound of formula 1-9;
Table 379 to Table 420: Includes all the compounds as disclosed in Table 1 to Table 42 respectively wherein compound of formula 1-1 is replaced by compound of formula 1-10;
Table 421 to Table 462: Includes all the compounds as disclosed in Table 1 to Table 42 respectively wherein compound of formula 1-1 is replaced by compound of formula 1-11;
Table 463 to Table 504: Includes all the compounds as disclosed in Table 1 to Table 42 respectively wherein compound of formula 1-1 is replaced by compound of formula 1-12;
Table 505 to Table 546: Includes all the compounds as disclosed in Table 1 to Table 42 respectively wherein compound of formula 1-1 is replaced by compound of formula 1-13;
Table 547 to Table 588: Includes all the compounds as disclosed in Table 1 to Table 42 respectively wherein compound of formula 1-1 is replaced by compound of formula 1-14;
Table 589 to Table 630: Includes all the compounds as disclosed in Table 1 to Table 42 respectively wherein compound of formula 1-1 is replaced by compound of formula 1-15;
Table 631 to Table 672: Includes all the compounds as disclosed in Table 1 to Table 42 respectively wherein compound of formula 1-1 is replaced by compound of formula 1-16;
Table 673 to Table 714: Includes all the compounds as disclosed in Table 1 to Table 42 respectively wherein compound of formula 1-1 is replaced by compound of formula 1-17;
Table 715 to Table 756: Includes all the compounds as disclosed in Table 1 to Table 42 respectively wherein compound of formula 1-1 is replaced by compound of formula 1-18;
Table 757 to Table 798: Includes all the compounds as disclosed in Table 1 to Table 42 respectively wherein compound of formula 1-1 is replaced by compound of formula 1-19;
Table 799 to Table 840: Includes all the compounds as disclosed in Table 1 to Table 42 respectively wherein compound of formula 1-1 is replaced by compound of formula 1-20.

In various embodiments, the compound of formula I is any one of compounds C-1 to C-66 as shown herein in the table in the examples section below.

As used herein, the term “compound(s) of the present invention” or “compound(s) according to the invention” refers to the compound(s) of formula (I) as defined above, which are also referred to as “compound(s) of formula I” or “compound(s) I” or “formula I compound(s)”, and includes their salts, tautomers, stereoisomers, and N-oxides.

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emusifier, dispersant, solubilizer, wetter, penetration enhancer, protective colloid, or adjuvant. Surfactants are listed in Mccutcheon's, Vol. 1: Emulsifiers & Detergents, Mccutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-substituted fatty acid amides, amine oxides, esters, sugar-based surfactants, polymeric surfactants. Suitable cationic surfactants are qua-ternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agro-chemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aero-sol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

### Pests

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne spp., Pratylenchus spp., Caenorhabditis elegans.

### Animal Health

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### EXAMPLES

With appropriate modification of the starting materials, the procedures as described in the preparation examples below were used to obtain further compounds of formula I. The compounds obtained in this manner are listed in the table C that follows, together with physical data.

Compounds can be characterized e.g., by coupled High Performance Liquid Chromatography/mass spectrometry (HPLC/MS), by 1H-NMR and/or by their melting points.

Analytical HPLC-Method 1: Agilent Eclipse Plus C18, 50×4.6 mm, ID 5 μm; Elution: A=10 mM Amm. Formate (0.1% Formic Acid), B=Acetonitrile (0.1% Formic Acid), Flow=1.2 ml/min. at 30° C.; Gradient: 10% B to 100% B-3 min, hold for 1 min, 1 min-10% B. Run Time=5.01 min.

Analytical HPLC-Method 2: Kinetex XB C18 1.7μ 50×2, 1 mm; A=Water+0.1% TFA,B=Acetonitrile, Flow=0.8 ml/min-1.0 ml/min in 1.5 min. at 60° C.; Gradient: 5% B to 100% B-1.5 min.

1H-NMR: The signals are characterized by chemical shift (ppm, δ [delta]) vs. tetramethylsilane respectively, CDCl₃ for ¹³C-NMR, by their multiplicity and by their integral (relative number of hydrogen atoms given). The following abbreviations are used to characterize the multiplicity of the signals: m=multiplet, q=quartet, t=triplet, d=doublet and s=singlet.

Abbreviations used are: d for day(s), h for hour(s), min for minute(s), ambient temperature is defined as 20-25° C., Rt for retention time; DMSO for dimethyl sulfoxide, OAc for acetate, EtOAc for ethyl acetate, THF for tetrahydrofuran, DMF for N, N-Dimethylformamide, CO for carbon monoxide, t-BuOH for tert-butanol and RH for relative humidity.

### Example C-1

### Synthesis of 1-[(E)-[4-[1,4-dimethyl-5-[1-oxo-5-(trifluoromethoxy)isoindolin-2-yl]pyrazol-3-yl]phenyl]methyleneamino]-3-(2-isopropyl-5-methyl-phenyl)thiourea

### Step 1: Synthesis of 3-(4-bromophenyl)-2-methyl-3-oxo-propanenitrile

Under an argon atmosphere, methyl 4-bromobenzoate (50 g, 232 mmol) and propionitrile (21.5 g, 390 mmol) were dissolved in tetrahydrofuran (500 ml) and a solution of lithium bis(trimethylsilyl)amide (370 ml, 1.1 M, 407 mmol) was added dropwise at ambient temperature. The reaction mixture was stirred overnight. The reaction was quenched by addition of water and extracted with dichloromethane. The organic phase was discarded. The aqueous phase was acidified with aqueous hydrochloric acid to pH 2 and extracted with dichloromethane (3×). The combined organic phase extracts were dried over sodium sulfate and concentrated. The desired product thus obtained (32 g, 57% yield) was used in the next step without further purification.

### Step 2: Synthesis of 5-(4-bromophenyl)-2,4-dimethyl-pyrazol-3-amine

Methylhydrazine sulfate (20 g, 175 mmol) was added to a stirred solution of 3-(4-bromophenyl)-2-methyl-3-oxo-propanenitrile (32 g, 134 mmol) in EtOH (3000 mL). Resulting mixture was stirred overnight at 80° C. and evaporated. Crude residue was treated with 2M NaOH to pH=9 filtered and concentrated under vacuum to give 5-(4-bromophenyl)-2,4-dimethyl-pyrazol-3-amine (22 g, 82 mmol, 47% yield) that was used in the next step without further purification.

### Step 3: Synthesis of methyl 2-methyl-4-(trifluoromethoxy)benzoate

Mixture of 2-methyl-4-(trifluoromethoxy)benzoic acid (30 g, 136 mmol), Mel (25.2 g, 177 mmol), and potassium carbonate (28.2 g, 204 mmol) in DMF (150 mL) was stirred under argon atmosphere at RT for 18 h. Obtained mixture was diluted with water (150 mL) and extracted with EtOAc (150 mL×3), Combined organic layers were washed with water (150 mL×2) and brine (200 mL), dried over sodium sulfate, filtered, and concentrated under vacuum affording methyl 2-methyl-4-(trifluoromethoxy)benzoate (31 g, 87% yield).

### Step 4: Synthesis of methyl 2-(bromomethyl)-4-(trifluoromethoxy)benzoate

2-methyl-4-(trifluoromethoxy)benzoate (30 g, 128 mmol) and benzoyl peroxide (BPO, 0.56 g, 2.3 mmol) was dissolved in CCl₄ (300 mL) and heated to reflux, NBS (25.2 g, 141 mmol) was added in portions and continue to heat reflux 2h, after cooling to ambient temperature, the reaction was filtered and concentrated under vacuum. The crude residue was purified by flash column chromatography to give methyl 2-(bromomethyl)-4-(trifluoromethoxy)benzoate (18 g, 44% yield).

### Step 5: Synthesis of 2-[5-(4-bromophenyl)-2,4-dimethyl-pyrazol-3-yl]-5-(trifluoromethoxy) isoindolin-1-one

Mixture of methyl 2-(bromomethyl)-4-(trifluoromethoxy)benzoate (10 g, 31 mmol), 5-(4-bromophenyl)-2,4-dimethyl-pyrazol-3-amine (8.5 g, 32 mmol) and N,N-diisopropylethylamine (33 ml, 186 mmol) in DMF (100 mL) was stirred under argon atmosphere at 80° C. for 18 h. Obtained mixture was diluted with water (100 mL) and extracted with EtOAc (100 mL×3), Combined organic layers were washed with water (50 mL×2) and brine (20 mL), dried over sodium sulfate, filtered, and concentrated under vacuum. The crude residue was purified by flash column chromatography to give 2-[5-(4-bromophenyl)-2,4-dimethyl-pyrazol-3-yl]-5-(trifluoromethoxy)isoindolin-1-one (6 g, 41% yield).

### Step 6: Synthesis of 2-[2,4-dimethyl-5-(4-vinylphenyl) pyrazol-3-yl]-5-(trifluoromethoxy)isoindolin-1-one

Pd2(dba)3 (0.23 g, 0.25 mmol) was added to a solution of 2-[5-(4-bromophenyl)-2,4-dimethyl-pyrazol-3-yl]-5-(trifluoromethoxy)isoindolin-1-one (11.25 g, 24 mmol), P(Cy)3 (0.18 phosphate (7.2 g, 33 mmol) and potassium g, 0.62 mmol), tripotassium ethenyltrifluoroboranide (6.7 g, 48 mmol) in dioxane (150 mL)/water (50 mL) mixture under argon atmosphere and the reaction mixture was stirred at 100° C. for 18 h. After it was cooled to ambient temperature, partitioned between EtOAc (150 mL) and water (100 mL) and the aqueous phase was extracted with EtOAc (100 mL×2). The combined organic layers were washed with brine (100 mL), dried over sodium sulfate, filtered, and concentrated under vacuum. The residue was subjected to flash column chromatography purification that afforded 2-[2,4-dimethyl-5-(4-vinylphenyl) pyrazol-3-yl]-5-(trifluoromethoxy)isoindolin-1-one (6 g, 58% yield).

### Step 7: Synthesis of 4-[1,4-dimethyl-5-[1-oxo-5-(trifluoromethoxy)isoindolin-2-yl]pyrazol-3-yl]benzaldehyde

A stirred solution of 8 (6 g, 14.27 mmol) in 1,4-dioxane (86 mL) was treated dropwise with water (57 mL), then sodium periodate (6 g, 30.0 mmol) and osmium tetroxide (0.2 g, 0.7 mmol). The reaction mixture was stirred at ambient temperature for 4 h and then concentrated. The residue was partitioned between water and EtOAc. The organic layer was washed with one portion of saturated aqueous sodium thiosulfate solution, one portion of brine, dried over sodium sulfate, filtered, and concentrated. The crude residue was purified by flash column chromatography to give 4-[1,4-dimethyl-5-[1-oxo-5-(trifluoromethoxy)isoindolin-2-yl]pyrazol-3-yl]benzaldehyde (1.8 g, 28% yield). ¹H NMR (400 MHz, Chloroform-d) δ 10.01 (s, 1H), 8.01 (s, 1H), 7.84 (d, 2H), 7.82 (d, 2H), 7.42-7.38 (m, 2H), 4.78 (s, 2H), 3.89 (s, 3H), 2.08 (s, 3H).

### Step 8: Synthesis of 1-[(E)-[4-[1,4-dimethyl-5-[1-oxo-5-(trifluoromethoxy)isoindolin-2-yl]pyrazol-3-yl]phenyl]methyleneamino]-3-(2-isopropyl-5-methyl-phenyl)thiourea (C-1)

To a stirred solution of 400 mg (0.96 mmol) 4-[1,4-dimethyl-5-[1-oxo-5-(trifluoromethoxy)isoindolin-2-yl]pyrazol-3-yl]benzaldehyde in 32 mL ethanol was added 258 mg (1.16 mmol, 2 equivalents) of 1-amino-3-(2-isopropyl-5-methyl-phenyl)thiourea. The solution was heated at 60° C. for 3h. Upon disappearance of the starting material as judged by TLC, the reaction mixture was cooled to ambient temperature, solvents evaporated and the crude reaction mixture subjected to flash chromatography to yield 510 mg (85% yield) of 1-[(E)-[4-[1,4-dimethyl-5-[1-oxo-5-(trifluoromethoxy)isoindolin-2-yl]pyrazol-3-yl]phenyl]methyleneamino]-3-(2-isopropyl-5-methyl-phenyl)thiourea. ¹H NMR (400 MHz, Chloroform-d) δ 9.59 (s, 1H), 8.98 (s, 1H), 8.04 (dd, J=7.9, 1.0 Hz, 1H), 7.90 (s, 1H), 7.79-7.68 (m, 4H), 7.47-7.40 (m, 2H), 7.27 (d, J=8.0 Hz, 1H), 7.18-7.11 (m, 1H), 4.77 (s, 2H), 3.81 (s, 3H), 3.14 (p, J=6.9 Hz, 1H), 2.37 (s, 3H), 2.15 (s, 3H), 1.32-1.20 (m, 6H).

### Example C-2

### Synthesis of (2Z)-2-[(E)-[4-[1,4-dimethyl-5-[1-oxo-5-(trifluoromethoxy)isoindolin-2-yl]pyrazol-3-yl]phenyl]methylenehydrazono]-3-(2-isopropyl-5-methyl-phenyl)thiazolidin-4-one

### Step 1: Synthesis of (2Z)-2-[(E)-[4-[1,4-dimethyl-5-[1-oxo-5-(trifluoromethoxy)isoindolin-2-yl]pyrazol-3-yl]phenyl]methylenehydrazono]-3-(2-isopropyl-5-methyl-phenyl)thiazolidin-4-one (C-2)

To a stirred solution of 300 mg (0.48 mmol) of 1-[(E)-[4-[1,4-dimethyl-5-[1-oxo-5-(trifluoromethoxy)isoindolin-2-yl]pyrazol-3-yl]phenyl]methyleneamino]-3-(2-isopropyl-5-methyl-phenyl)thiourea in 20 mL ethanol was first added 79 mg (1 mmol, 2 equivalents) sodium acetate, followed by 147 mg (1 mmol, 2 equivalents) methyl bromoacetate. The reaction mixture was heated at 60° C. for 2 h. Upon disappearance of starting material as judged by TLC, the solvents were evaporated and the crude mixture purified by flash chromatography to yield 262 mg (82%) of (2Z)-2-[(E)-[4-[1,4-dimethyl-5-[1-oxo-5-(trifluoromethoxy)isoindolin-2-yl]pyrazol-3-yl]phenyl]methylenehydrazono]-3-(2-isopropyl-5-methyl-phenyl)thiazolidin-4-one. ¹H NMR (400 MHz, Chloroform-d) δ 8.30 (s, 1H), 8.06-7.99 (m, 1H), 7.78 (d, J=8.4 Hz, 2H), 7.73 (d, J=8.4 Hz, 2H), 7.46-7.40 (m, 2H), 7.36 (d, J=8.0 Hz, 1H), 7.29 (dd, J=8.1, 1.9 Hz, 3H), 7.05-6.96 (m, 1H), 4.77 (s, 2H), 3.99 (d, J=1.7 Hz, 2H), 3.80 (s, 3H), 2.78 (hept, J=6.8 Hz, 1H), 2.37 (s, 3H), 2.14 (s, 3H), 1.22-1.20 (m, 6H).

### Example C-3

### Synthesis of [(Z)—N′—[(E)-[4-[1,4-dimethyl-5-[1-oxo-5-(trifluoromethoxy)isoindolin-2-yl]pyrazol-3-yl]phenyl]methyleneamino]-N-(2-isopropyl-5-methyl-phenyl) carbamimidoyl]sulfanylmethyl 2-methylpropanoate

### Step 1: Synthesis of [(Z)—N′—[(E)-[4-[1,4-dimethyl-5-[1-oxo-5-(trifluoromethoxy)isoindolin-2-yl]pyrazol-3-yl]phenyl]methyleneamino]-N-(2-isopropyl-5-methyl-phenyl) carbamimidoyl]sulfanylmethyl 2-methylpropanoate (C-3)

To a stirred solution of 250 mg (0.40 mmol) of 1-[(E)-[4-[1,4-dimethyl-5-[1-oxo-5-(trifluoromethoxy)isoindolin-2-yl]pyrazol-3-yl]phenyl]methyleneamino]-3-(2-isopropyl-5-methyl-phenyl)thiourea in 5 ml acetone was added 165 mg (1.20 mmol, 3 equivalents) of chloromethyl 2-methylpropanoate, 0.28 mL (1.61 mmol, 4 equivalents) of N,N-diisopropylamine and 18 mg (0.12 mmol, 0.3 equivalents) of sodium iodide. After stirring at 70° C. for 12 hours, the reaction mixture was cooled down to ambient temperature, solvents evaporated and the crude mixture purified by flash chromatography to yield 100 mg (35% yield) of [(Z)—N′—[(E)-[4-[1,4-dimethyl-5-[1-oxo-5-(trifluoromethoxy)isoindolin-2-yl]pyrazol-3-yl]phenyl]methyleneamino]-N-(2-isopropyl-5-methyl-phenyl) carbamimidoyl]sulfanylmethyl 2-methylpropanoate. ¹H NMR (500 MHz, Chloroform-d) δ 8.50 (s, 1H), 8.18 (s, 1H), 8.05-8.00 (m, 1H), 7.86-7.80 (m, 2H), 7.80-7.68 (m, 2H), 7.46-7.40 (m, 2H), 7.28-7.06 (m, 3H), 5.74 (s, 2H), 4.76 (s, 2H), 3.80 (s, 3H), 3.21 (hept, J=6.9 Hz, 1H), 2.58 (hept, J=7.0 Hz, 1H), 2.33 (s, 3H), 2.15 (s, 3H), 1.23 (d, J=6.9 Hz, 6H), 1.16 (d, J=7.0 Hz, 6H).

### Example C-4

### Synthesis of [(Z)—N′—[(E)-[4-[1,4-dimethyl-5-[1-oxo-5-(trifluoromethoxy)isoindolin-2-yl]pyrazol-3-yl]phenyl]methyleneamino]-N-(2-isopropyl-5-methyl-phenyl) carbamimidoyl]sulfanylmethyl ethyl carbonate

### Step 1: Synthesis of [(Z)—N′—[(E)-[4-[1,4-dimethyl-5-[1-oxo-5-(trifluoromethoxy)isoindolin-2-yl]pyrazol-3-yl]phenyl]methyleneamino]-N-(2-isopropyl-5-methyl-phenyl) carbamimidoyl]sulfanylmethyl ethyl carbonate (C-4)

To a stirred solution of 700 mg (1.12 mmol) of 1-[(E)-[4-[1,4-dimethyl-5-[1-oxo-5-(trifluoromethoxy)isoindolin-2-yl]pyrazol-3-yl]phenyl]methyleneamino]-3-(2-isopropyl-5-methyl-phenyl)thiourea in 5 ml acetone was added 467 mg (3.37 mmol, 3 equivalents) of chloromethyl-ethyl carbonate, 581 mg (4.50 mmol, 4 equivalents) of N,N-diisopropylamine and 50 mg (0.34 mmol, 0.3 equivalents) of sodium iodide. After stirring at 70° C. for 12 hours, the reaction mixture was cooled down to ambient temperature, solvents evaporated and the crude mixture purified by flash chromatography to yield 160 mg (18% yield) of [(Z)—N′—[(E)-[4-[1,4-dimethyl-5-[1-oxo-5-(trifluoromethoxy)isoindolin-2-yl]pyrazol-3-yl]phenyl]methyleneamino]-N-(2-isopropyl-5-methyl-phenyl) carbamimidoyl]sulfanylmethyl ethyl carbonate. ¹H NMR (400 MHz, Chloroform-d) δ 8.51 (s, 1H), 8.16 (s, 1H), 8.06-8.00 (m, 1H), 7.82 (d, J=8.2 Hz, 2H), 7.74 (d, J=8.2 Hz, 2H), 7.26-7.23 (m, 2H), 7.18-7.07 (m, 3H), 5.80 (s, 2H), 4.77 (s, 2H), 4.21 (q, J=7.1 Hz, 2H), 3.80 (s, 3H), 3.20 (h, J=6.8 Hz, 1H), 2.34 (3, 3H), 2.33 (s, 3H), 2.15 (s, 3H), 1.39-1.24 (m, 9H).

### Example C-7

### Synthesis of 1-[(E)-[4-[1,4-dimethyl-5-[1-oxo-6-(trifluoromethoxy)-3,4-dihydroisoquinolin-2-yl]pyrazol-3-yl]phenyl]methyleneamino]-3-(2-isopropyl-5-methyl-phenyl)thiourea

### Step 1: Synthesis of methyl 2-bromo-4-(trifluoromethoxy)benzoate

To a stirred solution of 2-bromo-1-iodo-4-(trifluoromethoxy)benzene (100 g, 272 mmol) in methanol (800 mL) mixture were added sodium methoxide 30% solution (73.62 g, 408 mmol) and 1,1′-bis(diphenyl phosphine) ferrocene-palladium (II) dichloride dichloromethane complex (2.26 g, 2.72 mmol) at ambient temperature. The whole reaction mixture was stirred at 27° C. for 16 hours under CO gas pressure (5 bar). The progress of the reaction was monitored by LCMS analysis. The reaction mixture was filtered through a celite pad and the filtrate was concentrated under reduced pressure to get the crude product. The crude product was purified by column chromatography using EtOAc and heptane as eluent to offer the desired product as colorless liquid (61.2 g, 75.2%). HPLC/MS: Rt: 2.16 min; m/z=301 (M+2)⁺.

### Step 2: Synthesis of methyl 4-(trifluoromethoxy)-2-vinyl-benzoate

Solution of methyl 2-bromo-4-(trifluoromethoxy)benzoate (39.0 g, 130.30 mmol) in THF (300 mL) and water (70 mL) was degassed with nitrogen gas for 30 min. To the stirred degassed solution were added potassium vinyl trifluoroborate (26.20 g, 195.63 mmol), cesium carbonate (131.72 g, 404.29 mmol), triphenylphosphine (3.42 g, 13.04 mmol) and palladium (II) chloride (1.15 g, 6.52 mmol) at ambient temperature. Reaction mass was heated for 12 h at 55° C. The progress of the reaction was monitored by TLC and LCMS. After the reaction was completed, the reaction mixture was filtered through a celite pad and washed with EtOAc (100 mL). The reaction Filtrate was diluted with water (200 mL) then extracted with EtOAc (100 mL×2). The combined organic layers were dried over sodium sulphate and concentrated under reduced pressure to get the crude product. The crude product was purified by column chromatography using EtOAc and heptane as eluent to offer the desired product as color less liquid (30.5 g, 94.9%). GC/MS: Rt: 4.57 min; m/z=246 (M).

### Step 3: Synthesis of 4-(trifluoromethoxy)-2-vinyl-benzoic acid

To a stirred solution of methyl 4-(trifluoromethoxy)-2-vinyl-benzoate (31.0 g, 125.92 mmol) in THF (260 mL) and water (50 mL) was added lithium hydroxide (15.07 g, 629.62 mmol) at ambient temperature. The reaction mixture was stirred at 27° C. for 16 h. The progress of the reaction was monitored by TLC. THF was evaporated through reduced pressure. Reaction mass was diluted with water (100 mL), extracted with EtOAc (2×100 mL). Aqueous layer was acidified with 2.0 N HCl solution (100 mL) to pH 3, followed by extraction with EtOAc (3×150 mL). Combined organic layers were dried over sodium sulphate and evaporated under reduced pressure to obtain the crude product as solid. The crude product was purified by column chromatography using EtOAc and heptane as eluent to afford the desired product as off-white solid (26.0 g, 89.6%). HPLC/MS (Method 1): Rt: 2.01 min; m/z=231.1 (M−1)+

### Step 4: Synthesis of 3-(4-bromophenyl)-2-methyl-3-oxo-propanenitrile

Under an argon atmosphere, ethyl 4-bromobenzoate (30 g, 130.9 mmol) and propionitrile (8.65 g, 157.16 mmol) were dissolved in THF (600 ml) and potassium tert-butoxide (29.33 g, 261.93 mmol) was added portion wise at ambient temperature. The reaction mixture was stirred at 27° C. for 4 h. The progress of the reaction was monitored by TLC. The reaction was quenched by addition of saturated ammonium chloride solution and followed by extraction with EtOAc (3×200 mL). Combined organic layers were dried over sodium sulphate and evaporated under reduced pressure to obtain the crude product as yellow solid (23.0 g) was used in the next step without further purification. ¹H NMR (500 MHz, CDCl₃) δ 7.91-7.84 (m, 2H), 7.73-7.67 (m, 2H), 4.33 (q, J=7.2 Hz, 1H), 1.66 (d, J=7.2 Hz, 3H).

### Step 5: Synthesis of 5-(4-bromophenyl)-2,4-dimethyl-pyrazol-3-amine

Methylhydrazine (6.67 g, 115.93 mmol, 80% in water) was added to a stirred solution of 3-(4-bromophenyl)-2-methyl-3-oxo-propanenitrile (23 g, 96.06 mmol) in ethanol (230 mL). Resulting mixture was stirred overnight at 80° C. and evaporated. Crude residue was treated with methyl tert-butyl ether, filtered, and concentrated under vacuum to give 5-(4-bromophenyl)-2,4-dimethyl-pyrazol-3-amine (20 g, 75% yield) that was used in the next step without further purification. HPLC/MS: Rt: 1.79 min; m/z=268 (M+2)⁺.

### Step 6: Synthesis of N-[5-(4-bromophenyl)-2,4-dimethyl-pyrazol-3-yl]-4-(trifluoromethoxy)-2-vinyl-benzamide

To a stirred solution of 5-(4-bromophenyl)-2,4-dimethyl-pyrazol-3-amine (10 g, 37.57 mmol) in dichloromethane (100 mL) was added 4-hydroxy-2-vinyl-benzoic acid (9.59 g, 41.33 mmol) followed by addition of N,N-diisopropylethylamine (19.4 mL, 112.72 mmol) at 10° C. The reaction mixture was cooled to 0° C. and to the reaction mixture was added 1-propanephosphonic anhydride solution, (67.07 mL, 50% solution in EtOAc, 112.72 mmol) dropwise over a period of 30 min. The reaction mixture was stirred at 27° C. for 16 h. The progress of the reaction was monitored by TLC. After completion of reaction, the reaction mixture was added to water dropwise (750 mL) and stirred for 60 min. White precipitate was formed which was filtered and dried under reduced pressure to obtain the desired product as white solid compound (15.2 g, 84%). HPLC/MS (Method 1): Rt: 2.29 min; m/z=483.2 (M+2)⁺.

### Step 7: Synthesis of 2-[5-(4-bromophenyl)-2,4-dimethyl-pyrazol-3-yl]-6-(trifluoromethoxy)-3,4-dihydroisoquinolin-1-one

To a stirred solution of N-[5-(4-bromophenyl)-2,4-dimethyl-pyrazol-3-yl]-4-(trifluoromethoxy)-2-vinyl-benzamide (1.0 g, 31.23 mmol) in dry DMF (8 mL) was added potassium tert-butoxide (0.07 g, 9.36 mmol) at ambient temperature. The reaction mixture was heated at 120° C. for 1 h under microwave irradiation. The progress of the reaction was monitored by TLC. The reaction mixture was diluted with water (100 mL) and extracted with EtOAc (50 mL×2). The combined organic layers were dried over sodium sulfate and concentrated under reduced pressure to get the crude product which was purified by column chromatography using EtOAc and heptane as eluent to afford the desired product as beige solid (0.48 g, 48%). HPLC/MS (Method 1): Rt: 2.35 min; m/z=482.1 (M+2)⁺.

### Step 8: Synthesis of 4-[1,4-dimethyl-5-[1-oxo-6-(trifluoromethoxy)-3,4-dihydroisoquinolin-2-yl]pyrazol-3-yl]benzaldehyde

To a stirred solution of 2-[5-(4-bromophenyl)-2,4-dimethyl-pyrazol-3-yl]-6-(trifluoromethoxy)-3,4-dihydroisoquinolin-1-one (1.1 g, 2.29 mmol) in dry DMF (25 mL) were added triethylsilane (0.79 g, 6.87 mmol), triethylamine (0.69 g, 6.87 mmol) and (1,3-Bis(diphenylphosphino) propane) palladium (II) chloride (0.20 g, 0.34 mmol) at ambient temperature. The reaction mixture was stirred at 80° C. for 16 h under CO atmosphere (9 bar) in autoclave. The progress of the reaction was monitored by TLC. The reaction mixture was diluted with water (100 mL) and extracted with EtOAc (75 mL×2). The combined organic layers were dried over sodium sulfate and concentrated under reduced pressure to get the crude product which was purified by column chromatography using EtOAc and heptane as eluent to afford the desired product as beige solid (0.9 g, 96% yield). ¹H NMR (300 MHz, DMSO-d6) δ 10.04 (s, 1H), 8.10 (d, J=8.6 Hz, 1H), 7.96 (q, J=8.3 Hz, 4H), 7.51 (s, 1H), 7.42 (d, J=8.5 Hz, 1H), 3.93 (t, J=6.5 Hz, 2H), 3.32 (t, J=6.5 Hz, 2H) 3.75 (s, 3H), 2.16 (s, 3H). HPLC/MS (Method 1): Rt: 2.07 min; m/z=430.3 (M+1)⁺.

### Step 9: Synthesis of 1-[(E)-[4-[1,4-dimethyl-5-[1-oxo-6-(trifluoromethoxy)-3,4-dihydroisoquinolin-2-yl]pyrazol-3-yl]phenyl]methyleneamino]-3-(2-isopropyl-5-methyl-phenyl)thiourea (C-7)

To a stirred solution of 4-[1,4-dimethyl-5-[1-oxo-6-(trifluoromethoxy)-3,4-dihydroisoquinolin-2-yl]pyrazol-3-yl]benzaldehyde (0.350 g, 0.815 mmol) in acetic acid (4 mL) was added 1-amino-3-(2-isopropylphenyl)thiourea (0.182 g, 0.815 mmol) at 0° C. The reaction mass was stirred for 3 h and the progress of the reaction was monitored by TLC analysis. After completion of reaction, reaction mixture was quenched in water (15 mL). The precipitated product was filtered through a filter paper and dried under reduced pressure to get the title compound as a solid 0.470 g (86.03% yield). LC/MS (Method 1): Rt: 2.34 min; m/z=635 (M+1)+; ¹H NMR (300 MHz, DMSO-d6) δ 11.69 (s, 1H), 9.88 (s, 1H), 8.10 (s, 1H), 8.02 (d, J=8.6 Hz, 1H), 7.89 (d, J=8.1 Hz, 2H), 7.65 (d, J=8.1 Hz, 2H), 7.43 (s, 1H), 7.35 (d, J=8.7 Hz, 1H), 7.16 (d, J=7.9 Hz, 1H), 7.04 (d, J=8.0 Hz, 1H), 6.96 (s, 1H), 3.84 (t, J=6.4 Hz, 2H), 3.65 (s, 3H), 3.21 (t, J=7.2 Hz, 2H), 3.02 (p, J=6.9 Hz, 1H), 2.22 (s, 3H), 2.05 (s, 3H), 1.10 (d, J=6.8 Hz, 6H).

### Example C-8

### Synthesis of (2Z)-2-[(E)-[4-[1,4-dimethyl-5-[1-oxo-6-(trifluoromethoxy)-3,4-dihydroisoquinolin-2-yl]pyrazol-3-yl]phenyl]methylenehydrazono]-3-(2-isopropyl-5-methyl-phenyl)thiazolidin-4-one

### Step 1: Synthesis of (2Z)-2-[(E)-[4-[1,4-dimethyl-5-[1-oxo-6-(trifluoromethoxy)-3,4-dihydroisoquinolin-2-yl]pyrazol-3-yl]phenyl]methylenehydrazono]-3-(2-isopropyl-5-methyl-phenyl)thiazolidin-4-one (C-8)

To a stirred solution of 1-[(E)-[4-[1,4-dimethyl-5-[1-oxo-6-(trifluoromethoxy)-3,4-dihydroisoquinolin-2-yl]pyrazol-3-yl]phenyl]methyleneamino]-3-(2-isopropyl-5-methyl-phenyl)thiourea (0.250 g, 0.393 mmol) in ethanol (5.0 mL) were added sodium acetate (0.065 g, 0.787 mmol) and methyl bromo acetate (0.090 g, 0.590 mmol) at ambient temperature. Then reaction mass was stirred at ambient temperature for 12 h and monitored by TLC analysis. After completion of reaction, reaction mass was diluted with water (20 mL) and followed by extracted in EtOAc (20 mL×2). The combined organic extracts were dried over sodium sulphate, filtered, and concentrated under reduced pressure. The crude product was purified by preparative HPLC to afford title compound as a solid 0.160 g (60.01% yield). LC/MS (Method 1): Rt: 2.38 min; m/z=675 (M+1)+; ¹H NMR (300 MHz, DMSO-d6) δ 8.36 (s, 1H), 8.10 (d, J=8.6 Hz, 1H), 7.86-7.72 (m, 4H), 7.50 (s, 1H), 7.46-7.35 (m, 2H), 7.29 (dd, J=8.4, 1.8 Hz, 1H), 7.11-7.04 (m, 1H), 4.25 (d, J=17.4 Hz, 1H), 4.13 (d, J=17.3 Hz, 1H), 3.92 (t, J=6.5 Hz, 2H), 3.73 (s, 3H), 3.22 (s, 2H), 2.75 (p, J=6.8 Hz, 1H), 2.32 (s, 3H), 2.12 (s, 3H), 1.13 (dd, J=9.3, 6.8 Hz, 6H).

### Example C-9

### Synthesis of (2Z)-3-(2-isopropyl-5-methyl-phenyl)-2-[(E)-[4-[1-methyl-5-[1-oxo-6-(trifluoromethoxy)-3,4-dihydroisoquinolin-2-yl]-1,2,4-triazol-3-yl]phenyl]methylenehydrazono]thiazolidin-4-one

### Step 1: Synthesis of 3,5-dibromo-1-methyl-1,2,4-triazole

To a stirred solution of 3,5-dibromo-1H-1,2,4-triazole (300 g, 1322 mmol) in 1,2 dichloroethane (1500 mL) mixture were added 2.0 M sodium hydroxide aqueous solution (750 mL, 1500 mmol), tetrabutylammonium hydrogen sulfate (44.8 g, 132.2 mmol) and dimethyl sulphate (175.13 g, 1388.5 mmol), at ambient temperature. The whole reaction mixture was stirred at 27° C. for 5 h. The progress of the reaction was monitored by HPLC analysis. The reaction mixture was diluted with water (500 mL) and extracted with DCM (500 mL×2). The combined organic layers were washed with saturated NaHCO₃ solution (1000 mL) dried over sodium sulfate and concentrated under reduced pressure to get the crude product which was purified by column chromatography using EtOAc and heptane as eluent to afford the desired product as beige solid (298 g, 93%). GCMS (Method 1): Rt: 4.68 min; m/z=241 (M).

### Step 2:5-bromo-N-[(4-methoxyphenyl)methyl]-2-methyl-1,2,4-triazol-3-amine

To a stirred solution of 3,5-dibromo-1-methyl-1,2,4-triazole (300 g, 1245.4 mmol) in N-methylpyrrolidone (3000 mL), were added 4-methoxy benzylamine (179.3 g, 1307.7 mmol) and potassium carbonate (258.1 g, 1868.2 mmol) at ambient temperature. The whole reaction mixture was stirred at 120° C. for 16 h. The progress of the reaction was monitored by HPLC and LCMS. After the reaction was completed, the reaction mixture was added drop wise into water (10000 mL) under stirring over 45 min. White precipitate was formed which was filtered and dried under reduced pressure to obtain the desired product as white solid (285 g, 77%). LC/MS: Rt: 1.82 min; m/z=301 (M+2)+.

### Step 3: Synthesis of 4-[5-[(4-methoxyphenyl)methylamino]-1-methyl-1,2,4-triazol-3-yl]benzaldehyde

Solution of 5-bromo-N-[(4-methoxyphenyl)methyl]-2-methyl-1,2,4-triazol-3-amine (300.0 g, 1009.6 mmol) in 1,4-Dioxane (2500 mL) and water (500 mL) was degassed with nitrogen gas for 30 min. To the stirred and degassed solution were followed added 4-formylphenyl boronic acid (158.9 g, 1060.1 mmol), cesium carbonate (657.89 g, 2019.2 mmol), [1,1′-bis(diphenylphosphino) ferrocene]dichloropalladium (II) (14.7 g, 20.192 mmol) at ambient temperature. Reaction mass was heated for 6 hours at 90° C. The progress of the reaction was monitored by HPLC and LCMS. After the completion of reaction, the reaction mixture was filtered through a celite pad and washed with EtOAc (1000 mL). The combined organic layers were washed with saturated sodium bicarbonate solution (1000 mL), dried over sodium sulfate, and concentrated under reduced pressure to get the crude product (brown solid). Crude solid was treated with methanol (300 mL×2) and filtered to afford the desired product as pale-yellow solid (295 g, 77%). HPLC/MS (Method 1): Rt: 1.91 min; m/z=323.2 (M+1)+.

### Step 4: Synthesis of 4-(5-amino-1-methyl-1,2,4-triazol-3-yl)benzaldehyde

To a stirred solution of 4-[5-[(4-methoxyphenyl)methylamino]-1-methyl-1,2,4-triazol-3-yl]benzaldehyde (50 g, 155.1 mmol) in 1,2 dichloromethane (500 mL) was added triflic acid (23.27 g, 155.1 mmol) at 0° C. The reaction mixture was stirred at 27° C. for 3 h. The progress of the reaction was monitored by LCMS and HPLC. After completion of reaction, the reaction mixture was dosed into an aqueous potassium carbonate solution (1.5 eq, 500 mL) over 30 min. White precipitate was formed which was filtered and dried under reduced pressure to obtain the desired product as white solid (28.2 g, 90%). ¹H NMR (500 MHz, DMSO) δ 10.01 (s, 1H), 8.09-8.04 (m, 2H), 7.96-7.91 (m, 2H), 6.41 (s, 2H), 3.62 (s, 3H): Rt: 1.62 min; m/z=203.1 (M+1)⁺.

### Step 5: Synthesis of (2Z)-2-[(E)-[4-(5-amino-1-methyl-1,2,4-triazol-3-yl)phenyl]methylenehydrazono]-3-(2-isopropyl-5-methyl-phenyl)thiazolidin-4-one

To a stirred solution of 4-(5-amino-1-methyl-1,2,4-triazol-3-yl)benzaldehyde (4.0 g, 19.78 mmol) in DMF (40 mL) was added 1-amino-3-(2-isopropyl-5-methyl-phenyl)thiourea (4.6 g, 20.77 mmol). The solution was stirred at 27° C. for a period of 3 h. Upon disappearance of the starting material as judged by HPLC and LCMS, sodium acetate (2.43 g, 29.62 mmol) followed by methyl bromoacetate (3.32 g, 21.76 mmol) were added. The reaction mixture was heated at 60° C. for 16 h. Upon disappearance of starting material as judged by HPLC and LCMS, the reaction mixture was added dropwise to water (750 mL) and stirred for 15 min. White precipitate was formed which was filtered and dried under reduced pressure to obtain the desired product as white solid compound (7.1 g, 80% yield). ¹H NMR (500 MHz, DMSO) δ 8.33 (s, 1H), 7.95-7.89 (m, 2H), 7.78-7.73 (m, 2H), 7.38 (d, J=8.0 Hz, 1H), 7.28 (dd, J=8.2, 1.9 Hz, 1H), 7.09-7.05 (m, 1H), 6.35 (s, 2H), 4.24 (d, J=17.3 Hz, 1H), 4.13 (d, J=17.3 Hz, 1H), 3.59 (s, 3H), 2.73 (q, J=6.5 Hz, 1H), 2.32 (s, 3H), 1.12 (dd, J=13.1, 6.8 Hz, 6H). HPLC/MS (Method 1): Rt: 1.99 min; m/z=448.3 (M+1)⁺.

### Step 6: Synthesis of N-[5-[4-[(E)-[(Z)-[3-(2-isopropyl-5-methyl-phenyl)-4-oxo-thiazolidin-2-ylidene]hydrazono]methyl]phenyl]-2-methyl-1,2,4-triazol-3-yl]-4-(trifluoromethoxy)-2-vinyl-benzamide

To a stirred solution of (2Z)-2-[(E)-[4-(5-amino-1-methyl-1,2,4-triazol-3-yl)phenyl]methylenehydrazono]-3-(2-isopropyl-5-methyl-phenyl)thiazolidin-4-one (0.3 g, 0.67 mmol) and 4-(trifluoromethoxy)-2-vinyl-benzoic acid (0.187 g, 0.80 mmol) in pyridine (3 mL) at 0° C. was added phosphorus oxychloride (0.125 ml, 1.34 mmol). The reaction mixture was stirred at 27° C. for 15 min. The progress of the reaction was monitored by TLC. After completion of reaction, the reaction mixture was quenched with methanol (0.5 mL) and concentrated under reduced pressure to get the crude product which was purified by column chromatography using EtOAc and heptane as eluent. Product obtained was dissolved in methanol (10 ml) and potassium carbonate (0.278 g, 2.01 mmol) was added and stirred for 5 min. The reaction mixture was diluted with water (50 mL) and was extracted with EtOAc (50 mL×2). The combined organic layers were dried over sodium sulfate and concentrated under reduced pressure to get the crude product which was purified by column chromatography using EtOAc and heptane as eluent to afford the desired product as beige solid (0.25 g, 53%). HPLC/MS (Method 1): Rt: 2.29 min; m/z=660 (M+1)⁺.

### Step 7: Synthesis of (2Z)-3-(2-isopropyl-5-methyl-phenyl)-2-[(E)-[4-[1-methyl-5-[1-oxo-6-(trifluoromethoxy)-3,4-dihydroisoquinolin-2-yl]-1,2,4-triazol-3-yl]phenyl]methylenehydrazono]thiazolidin-4-one (C-9)

To a stirred solution of N-[5-[4-[(E)-[(Z)-[3-(2-isopropyl-5-methyl-phenyl)-4-oxo-thiazolidin-2-ylidene]hydrazono]methyl]phenyl]-2-methyl-1,2,4-triazol-3-yl]-4-(trifluoromethoxy)-2-vinyl-benzamide (0.2 g, 0.3 mmol) in dry DMF (5 mL) was added potassium tert-butoxide (0.027 g, 0.24 mmol) at ambient temperature. The reaction mixture was heated at 100° C. for 48 h. The progress of the reaction was monitored by TLC. The reaction mixture was diluted with water (50 mL) and extracted with EtOAc (50 mL×2). The combined organic layers were dried over sodium sulfate and concentrated under reduced pressure to get the crude product which was purified by column chromatography using EtOAc and heptane as eluent to afford the desired product as beige solid (0.065 g, 29%). HPLC/MS (Method 1): Rt: 2.37 min; m/z=662.3 (M+1)+. ¹H NMR (300 MHz, DMSO-d6) δ 8.37 (s, 1H), 8.12 (d, J=8.6 Hz, 1H), 8.04 (d, J=8.1 Hz, 2H), 7.84 (d, J=8.3 Hz, 2H), 7.52 (d, J=2.5 Hz, 1H), 7.47-7.35 (m, 2H), 7.28 (d, J=8.1 Hz, 1H), 7.08 (d, J=1.7 Hz, 1H), 4.30-4.04 (m, 4H), 3.77 (s, 3H), 3.27 (m, 2H), 2.74 (t, J=6.8 Hz, 1H), 2.32 (s, 3H), 1.19-1.05 (m, 6H).

### Example C-11

### Synthesis of (3Z)-1-[4-[1,4-dimethyl-5-[1-oxo-6-(trifluoromethoxy)-3,4-dihydroisoquinolin-2-yl]pyrazol-3-yl]phenyl]-3-[3-(2-isopropyl-5-methyl-phenyl)-4-oxo-thiazolidin-2-ylidene]urea

### Step 1: Synthesis of 2-[5-(4-aminophenyl)-2,4-dimethyl-pyrazol-3-yl]-6-(trifluoromethoxy)-3,4-dihydroisoquinolin-1-one

A solution of 2-[5-(4-bromophenyl)-2,4-dimethyl-pyrazol-3-yl]-6-(trifluoromethoxy)-3,4-dihydroisoquinolin-1-one (7.1 g, 14.78 mmol) and boc-amide (2.59 g, 22.17 mmol) in 1,4-dioxane (80 mL) was degassed with nitrogen gas for 30 min. To the stirred and degassed solution were added potassium tert-butoxide (3.31 g, 29.56 mmol), Tris(dibenzylideneacetone)dipalladium (0.677 g, 0.73 mmol), 5-(di-tert-butylphosphino)-1′,3′,5′-triphenyl-1′H-[1,4′]bipyrazole (0.749 g, 1.47 mmol) at ambient temperature. Reaction mass was heated for 12 h at 90° C. The progress of the reaction was monitored by TLC and LCMS. After the reaction was complete, the reaction mixture was filtered through a celite pad and washed with EtOAc (100 mL). Filtrate was concentrated to its one-third volume and extracted with EtOAc (2×100 mL). Organic layers were dried over sodium sulphate and evaporated under reduced pressure to obtain the crude product as off-white solid (7.1 g). To this crude compound was added dichloromethane (70 mL) followed by dropwise addition of 4.0 M HCl in 1,4 dioxane (10.23 mL, 40.94 mmol) at 10° C. Reaction mass was stirred for 12 hours at ambient temperature. The progress of the reaction was monitored by TLC and LCMS. After the completion of the reaction, it was added to water (100 mL) dropwise under stirring, extracted with EtOAc (2×50 mL). Aqueous layer was basified with saturated sodium carbonate solution (100 mL) followed by extraction with EtOAc (3×100 mL). Combined organic layers were dried over sodium sulphate and evaporated under reduced pressure to obtain the crude product as off-white solid (5.2 g). The crude product was purified by column chromatography using EtOAc and heptane as eluent to afford the desired product as off-white solid (4.6 g, 71.2%). HPLC/MS (Method 1): Rt: 1.93 min; m/z=417 (M+1)⁺.

### Step 2: Synthesis of (3Z)-1-[4-[1,4-dimethyl-5-[1-oxo-6-(trifluoromethoxy)-3,4-dihydroisoquinolin-2-yl]pyrazol-3-yl]phenyl]-3-[3-(2-isopropyl-5-methyl-phenyl)-4-oxo-thiazolidin-2-ylidene]urea (C-11)

To a solution of 2-[5-(4-aminophenyl)-2,4-dimethyl-pyrazol-3-yl]-6-(trifluoromethoxy)-3,4-dihydroisoquinolin-1-one (0.15 g, 0.360 mmol) in dry acetonitrile (5 mL) was added pyridine (0.116 mL, 1.44 mmol) at ambient temperature. The reaction mixture was cooled to 0° C. and N,N′-disuccinimidyl carbonate (0.097 g, 0.378 mmol) was added at 0° C. The reaction mixture was allowed to stir at ambient temperature for 3 h. The progress of the reaction was monitored by TLC. Pyridine (0.116 mL, 1.44 mmol) followed by 2-imino-3-(2-isopropyl-5-methyl-phenyl) thiazolidin-4-one (0.089 g, 0.360 mmol) was added to the reaction mixture and stirred at 60° C. for 3 h. The progress of the reaction was monitored by TLC. The reaction mixture was diluted with water (30 mL) and extracted with EtOAc (50 mL×2). The combined organic layers were dried over sodium sulfate and concentrated under reduced pressure to get the crude product which was purified by column chromatography using EtOAc and heptane as eluent to afford the desired product as beige solid (0.175 g). HPLC/MS (Method 1): Rt: 2.24 min; m/z=691 (M+1)+. ¹H NMR (500 MHz, DMSO-d6) δ 9.86 (s, 1H), 8.09 (d, J=8.6 Hz, 1H), 7.76-7.67 (m, 2H), 7.56 (d, J=8.8 Hz, 2H), 7.50 (s, 1H), 7.41 (t, J=7.5 Hz, 2H), 7.28 (d, J=8.0 Hz, 1H), 7.07 (s, 1H), 4.20 (d, J=17.7 Hz, 1H), 4.08 (d, J=18.1 Hz, 1H), 3.89 (t, J=6.5 Hz, 2H), 3.68 (s, 3H), 3.33-3.23 (m, 2H), 2.72-2.63 (m, 1H), 2.32 (s, 3H), 2.05 (s, 3H), 1.18 (d, J=6.9 Hz, 3H), 1.10 (d, J=6.8 Hz, 3H).

### Example C-12

### Synthesis of (3Z)-1-[2-chloro-4-[1,4-dimethyl-5-[1-oxo-6-(trifluoromethoxy)-3,4-dihydroisoquinolin-2-yl]pyrazol-3-yl]phenyl]-3-[3-(2-isopropyl-5-methyl-phenyl)-4-oxo-thiazolidin-2-ylidene]urea

### Step 1: Synthesis of 2-[5-(4-amino-3-chloro-phenyl)-2,4-dimethyl-pyrazol-3-yl]-6-(trifluoromethoxy)-3,4-dihydroisoquinolin-1-one

To a stirred solution of 2-[5-(4-aminophenyl)-2,4-dimethyl-pyrazol-3-yl]-6-(trifluoromethoxy)-3,4-dihydroisoquinolin-1-one (0.25 g, 0.600 mmol) in acetonitrile (10 mL) was added N-chlorosuccinimide (0.080 g, 0.600 mmol) at ambient temperature. The reaction mixture was heated at 70° C. for 7 h. The progress of the reaction was monitored by TLC. The reaction mixture was diluted with saturated sodium bicarbonate solution (20 mL) and extracted with EtOAc (50 mL×2). The combined organic layers were dried over sodium sulfate and concentrated under reduced pressure to get the crude product which was purified by column chromatography using EtOAc and heptane as eluent to afford the desired product as beige solid (0.22 g). HPLC/MS (Method 1): Rt: 2.10 min; m/z=451 (M+1)+. ¹H NMR (500 MHz, DMSO-d6) δ 8.01 (d, J=8.6 Hz, 1H), 7.43 (s, 1H), 7.39 (d, J=2.0 Hz, 1H), 7.34 (dd, J=8.6, 1.5 Hz, 1H), 7.27 (dd, J=8.4, 2.0 Hz, 1H), 6.78 (d, J=8.4 Hz, 1H), 5.39 (s, 2H), 3.81 (t, J=6.5 Hz, 2H), 3.59 (s, 3H), 3.25-3.13 (m, 2H), 1.96 (s, 3H).

### Step 2: Synthesis of (3Z)-1-[2-chloro-4-[1,4-dimethyl-5-[1-oxo-6-(trifluoromethoxy)-3,4-dihydroisoquinolin-2-yl]pyrazol-3-yl]phenyl]-3-[3-(2-isopropyl-5-methyl-phenyl)-4-oxo-thiazolidin-2-ylidene]urea (C-12)

To a solution of 2-[5-(4-amino-3-chloro-phenyl)-2,4-dimethyl-pyrazol-3-yl]-6-(trifluoromethoxy)-3,4-dihydroisoquinolin-1-one (0.220 g, 0.488 mmol) in dry acetonitrile (10 mL) was added pyridine (0.158 mL, 1.951 mmol) at ambient temperature. The reaction mixture was cooled to 0° C. and N,N′-disuccinimidyl carbonate (0.131 g, 0.512 mmol) was added. The reaction mixture was allowed to stir at ambient temperature for 3 h. The progress of the reaction was monitored by TLC. Pyridine (0.158 mL, 1.951 mmol) followed by 2-imino-3-(2-isopropyl-5-methyl-phenyl)thiazolidin-4-one (0.122 g, 0.488 mmol) was added to the reaction mixture and stirred at 60° C. for 3 h. The progress of the reaction was monitored by TLC. The reaction mixture was diluted with water (30 mL) and was extracted with EtOAc (30 mL×2). The combined organic layers were dried over sodium sulfate and concentrated under reduced pressure to get the crude product which was purified by column chromatography using EtOAc and heptane as eluent to afford the desired product as beige solid (0.175 g). HPLC/MS (Method 1): Rt: 2.36 min; m/z=725 (M+1)⁺. ¹H NMR (500 MHz, DMSO-d6) δ 9.00 (s, 1H), 8.02 (d, J=8.6 Hz, 1H), 7.68-7.48 (m, 3H), 7.43 (s, 1H), 7.38-7.30 (m, 2H), 7.21 (d, J=8.0 Hz, 1H), 7.00 (s, 1H), 4.14 (d, J=18.0 Hz, 1H), 4.02 (d, J=18.0 Hz, 1H), 3.84 (t, J=6.5 Hz, 2H), 3.64 (s, 3H), 3.24-3.17 (m, 2H), 2.66-2.56 (m, 1H), 2.25 (s, 3H), 2.02 (s, 3H), 1.12 (d, J=6.3 Hz, 3H), 1.03 (d, J=6.8 Hz, 3H).

### Example C-17

### Synthesis of 1-[(E)-[4-[3,5-dimethyl-4-[1-oxo-6-(trifluoromethoxy)-3,4-dihydroisoquinolin-2-yl]pyrazol-1-yl]-3-fluoro-phenyl]methyleneamino]-3-(2-isopropyl-5-methyl-phenyl)thiourea

### Step-1: Synthesis of N-(3,5-dimethyl-1H-pyrazol-4-yl)-4-(trifluoromethoxy)-2-vinyl-benzamide

To a stirred solution of 3,5-dimethyl-1H-pyrazol-4-amine (7.66 g, 68.92 mmol) in dichloromethane (160 mL) was added 4-(trifluoromethoxy)-2-vinyl-benzoic acid (16.0 g, 68.92 mmol) followed by addition of N,N, N-triethylamine (19.21 mL, 137.84 mmol) at 25° C. The reaction mixture was cooled to −7° C. and to the reaction mixture was added 1-propanephosphonic anhydride solution, (52.62 g, 50% solution in EtOAc, 82.7 mmol) dropwise over a period of 2 h. The reaction mixture was stirred at 25° C. for 19 h. The progress of the reaction was monitored by TLC. After completion of reaction, the reaction mixture was added to water dropwise (600 mL). Precipitate was formed which was filtered and dried under reduced pressure to obtain the desired product as solid compound (18.5 g, 82.55%). HPLC/MS (Method 1): Rt: 1.83 min; m/z=326.2 (M+1)+.

### Step-2: Synthesis of N-(3,5-dimethyl-1-tetrahydropyran-2-yl-pyrazol-4-yl)-4-(trifluoromethoxy)-2-vinyl-benzamide

To a solution of N-(3,5-dimethyl-1H-pyrazol-4-yl)-4-(trifluoromethoxy)-2-vinyl-benzamide (18 g, 55.34 mmol) in tetrahydrofuran (210 mL) were added PTSA (0.953 g, 5.53 mmol) and 3,4-dihydro-2H-pyran (6.982 g, 83 mmol) at ambient temperature. The reaction mixture was stirred to 60° C. for 6 h. The progress of the reaction was monitored by TLC. The reaction mixture was diluted with Aq. 2% sodium bicarbonate solution (150 mL) and was extracted with DCM (150 mL×2). The combined organic layers were dried over sodium sulfate and concentrated under reduced pressure to get the desired product as solid (16.0 g, 60.73% yield). HPLC/MS (Method 1): Rt: 2.02 min; m/z=408.3 (M−1).

### Step-3: Synthesis of 2-(3,5-dimethyl-1-tetrahydropyran-2-yl-pyrazol-4-yl)-6-(trifluoromethoxy)-3,4-dihydroisoquinolin-1-one

To a stirred solution of N-(3,5-dimethyl-1-tetrahydropyran-2-yl-pyrazol-4-yl)-4-(trifluoromethoxy)-2-vinyl-benzamide (17.6 g, 42.99 mmol) in DMF (200 mL) were added potassium tert-butoxide (2.412 g, 21.49 mmol) and scandium trifluoromethane sulfonate (1.058 g, 2.15 mmol) at ambient temperature. The reaction mixture was heated at 120° C. for 16 h. The progress of the reaction was monitored by TLC. The reaction mixture was diluted with water (1200 mL) and was extracted with EtOAc (400 mL×2). The combined organic layers were dried over sodium sulfate and concentrated under reduced pressure to get the crude product which was used in next step without further purification (17 g, 86.93% yield). HPLC/MS (Method 1): Rt: 2.06 min; m/z=410.3 (M+1)⁺.

### Step-4: Synthesis of 2-(3,5-dimethyl-1H-pyrazol-4-yl)-6-(trifluoromethoxy)-3,4-dihydroisoquinolin-1-one

To a stirred solution of 2-(3,5-dimethyl-1-tetrahydropyran-2-yl-pyrazol-4-yl)-6-(trifluoromethoxy)-3,4-dihydroisoquinolin-1-one (17 g, 41.52 mmol) in dichloromethane (200 mL) was added 4N HCl in Dioxane (20.76 mL, 83.05 mmol) dropwise over a period of 10 min at 0° C. The reaction mixture was stirred at ambient temperature for 16 h. The progress of the reaction was monitored by TLC. After completion of reaction, reaction mass was diluted with saturated Aq. sodium bicarbonate solution (300 mL) and was extracted with DCM (300 mL×2). The combined organic layers were dried over sodium sulfate and concentrated under reduced pressure to obtain the desired product as solid compound (9.3 g, 58.52% yield). HPLC/MS (Method 1): Rt: 1.84 min; m/z=326.2 (M+1)+. ¹H NMR (300 MHz, DMSO) δ 12.24 (s, 1H), 8.03 (d, J=8.5 Hz, 1H), 7.42 (d, J=2.4 Hz, 1H), 7.36 (dd, J=8.5, 2.4 Hz, 1H), 3.74 (t, J=6.4 Hz, 2H), 3.17 (t, J=6.5 Hz, 2H), 2.06 (d, J=13.3 Hz, 6H).

### Step-5: Synthesis of 4-[3,5-dimethyl-4-[1-oxo-6-(trifluoromethoxy)-3,4-dihydroisoquinolin-2-yl]pyrazol-1-yl]-3-fluoro-benzaldehyde

To a stirred solution of 2-(3,5-dimethyl-1H-pyrazol-4-yl)-6-(trifluoromethoxy)-3,4-dihydroisoquinolin-1-one (1.5 g, 4.61 mmol) in DMF (30 mL) were added 3,4-difluorobenzaldehyde (0.721 g, 5.07 mmol) and cesium carbonate (3.007 g, 9.22 mmol) at ambient temperature. The reaction mixture was heated at 110° C. for 6 h. The progress of the reaction was monitored by TLC. The reaction mixture was diluted with water (40 mL) and extracted with EtOAc (50 mL×2). The combined organic layers were dried over sodium sulfate and concentrated under reduced pressure to get the crude product which was purified by column chromatography using EtOAc and heptane as eluent to afford the desired product as beige solid (0.710 g, 34.41% yield). HPLC/MS (Method 1): Rt: 2.08 min; m/z=448.2 (M+1)⁺.

### Step-6: Synthesis of 1-[(E)-[4-[3,5-dimethyl-4-[1-oxo-6-(trifluoromethoxy)-3,4-dihydroisoquinolin-2-yl]pyrazol-1-yl]-3-fluoro-phenyl]methyleneamino]-3-(2-isopropyl-5-methyl-phenyl)thiourea (C-17)

To a stirred solution of 4-[3,5-dimethyl-4-[1-oxo-6-(trifluoromethoxy)-3,4-dihydroisoquinolin-2-yl]pyrazol-1-yl]-3-fluoro-benzaldehyde (0.700 g, 1.56 mmol) in ethanol (7 mL) and acetic acid (0.5 mL) was added 1-amino-3-(2-isopropyl-5-methyl-phenyl)thiourea (0.349 g, 1.56 mmol) at 0° C. The reaction mass was stirred at 75° C. for 3 h, and the progress of the reaction was monitored by TLC analysis. After completion of reaction, reaction mixture was quenched in water (15 mL). The precipitated product was filtered through a filter paper and dried under reduced pressure to get the title compound as a solid 0.890 g (87.15% yield). LC/MS (Method 1): Rt: 2.32 min; m/z=653.3 (M+1)+; ¹H NMR (300 MHz, DMSO) δ 11.94 (s, 1H), 10.15 (s, 1H), 8.35-8.25 (m, 1H), 8.17 (s, 1H), 8.06 (d, J=8.6 Hz, 1H), 7.75 (dd, J=8.3, 1.7 Hz, 1H), 7.57 (t, J=8.0 Hz, 1H), 7.46 (s, 1H), 7.38 (d, J=8.6 Hz, 1H), 7.25 (d, J=7.9 Hz, 1H), 7.13 (d, J=7.9 Hz, 1H), 6.98 (s, 1H), 3.84 (t, J=6.5 Hz, 2H), 3.22 (t, J=6.5 Hz, 2H), 3.07 (p, J=6.9 Hz, 1H), 2.29 (s, 3H), 2.16-2.05 (m, 6H), 1.17 (d, J=6.9 Hz, 6H).

### Example C-18

### Synthesis of (2Z)-2-[(E)-[4-[3,5-dimethyl-4-[1-oxo-6-(trifluoromethoxy)-3,4-dihydroisoquinolin-2-yl]pyrazol-1-yl]-3-fluoro-phenyl]methylenehydrazono]-3-(2-isopropyl-5-methyl-phenyl)thiazolidin-4-one (C-18)

To a stirred solution of 1-[(E)-[4-[3,5-dimethyl-4-[1-oxo-6-(trifluoromethoxy)-3,4-dihydroisoquinolin-2-yl]pyrazol-1-yl]-3-fluoro-phenyl]methyleneamino]-3-(2-isopropyl-5-methyl-phenyl)thiourea (0.690 g, 1.06 mmol) in ethanol (6.9 mL) were added sodium acetate (0.173 g, 2.11 mmol) and methyl bromo acetate (0.243 g, 1.59 mmol) at ambient temperature. Then reaction mass was stirred at ambient temperature for 20 h and monitored by TLC analysis. After completion of reaction, reaction mass was diluted with water (20 mL) and the precipitated product was filtered through a filter paper and dried under reduced pressure to get the title compound as a solid 0.652 g (89.03% yield). LC/MS (Method 1): Rt: 2.36 min; m/z=693.3 (M+1)+; ¹H NMR (300 MHz, DMSO) δ 8.43 (s, 1H), 8.06 (d, J=8.6 Hz, 1H), 7.81-7.70 (m, 2H), 7.65 (t, J=8.0 Hz, 1H), 7.46 (s, 1H), 7.39 (d, J=7.8 Hz, 2H), 7.28 (d, J=8.4 Hz, 1H), 7.08 (s, 1H), 4.28 (d, J=17.4 Hz, 1H), 4.15 (d, J=17.4 Hz, 1H), 3.84 (t, J=6.4 Hz, 2H), 3.22 (t, J=6.5 Hz, 2H), 2.73 (p, J=6.8 Hz, 1H), 2.32 (s, 3H), 2.20-2.01 (m, 6H), 1.12 (t, J=7.4 Hz, 6H).

### Example C-32

### Synthesis of (1Z)-1-[3-(2-isopropyl-5-methyl-phenyl)-4-oxo-thiazolidin-2-ylidene]-3-[4-[3-methyl-4-[1-oxo-6-(trifluoromethoxy)-3,4-dihydroisoquinolin-2-yl]pyrazol-1-yl]phenyl]urea

### Step-1: Synthesis of 2-[1-(4-aminophenyl)-3-methyl-pyrazol-4-yl]-6-(trifluoromethoxy)-3,4-dihydroisoquinolin-1-one

To a solution of 2-(3-methyl-1H-pyrazol-4-yl)-6-(trifluoromethoxy)-3,4-dihydroisoquinolin-1-one (0.600 g, 1.93 mmol) in 1,2-dichloroethane (6 mL) were added 4-(4,4,5,5-tetramethyl-1,3,2-dioxaborolan-2-yl) aniline (0.633 g, 2.89 mmol) and copper acetate (0.7 g, 3.86 mmol) at ambient temperature. Then to a reaction mass were added 2,2-bipyridyl (0.602 g, 3.86 mmol) and sodium carbonate (0.409 g, 3.86 mmol) at ambient temperature. The reaction mixture was stirred to 70° C. for 18 h. The progress of the reaction was monitored by TLC. The reaction mixture was diluted with water (25 mL) and was extracted with EtOAc (30 ml×2). The combined organic layers were dried over sodium sulfate and concentrated under reduced pressure to get the crude product which was purified by column chromatography using EtOAc and heptane as eluent to afford the desired product as beige solid (0.440 g) (56.72% yield). HPLC/MS (Method 1): Rt: 1.94 min; m/z=403.3 (M+1)⁺.

### Step-2: (1Z)-1-[3-(2-isopropyl-5-methyl-phenyl)-4-oxo-thiazolidin-2-ylidene]-3-[4-[3-methyl-4-[1-oxo-6-(trifluoromethoxy)-3,4-dihydroisoquinolin-2-yl]pyrazol-1-yl]phenyl]urea (C-32)

To a solution of 2-[1-(4-aminophenyl)-3-methyl-pyrazol-4-yl]-6-(trifluoromethoxy)-3,4-dihydroisoquinolin-1-one (0.200 g, 0.500 mmol) in dry acetonitrile (2 mL) was added pyridine (0.158 g, 1.99 mmol) at ambient temperature. The reaction mixture was cooled to 0° C. and N, N′-disuccinimidyl carbonate (0.140 g, 0.550 mmol) was added. The reaction mixture was allowed to stir at ambient temperature for 3 h. The progress of the reaction was monitored by TLC. Pyridine (0.158 g, 1.99 mmol) followed by 2-imino-3-(2-isopropyl-5-methyl-phenyl) thiazolidin-4-one (0.136 g, 0.55 mmol) were added to the reaction mixture and stirred at 65° C. for 3 h. The progress of the reaction was monitored by TLC. The reaction mixture was diluted with water (20 mL) and was extracted with EtOAc (25 mL×2). The combined organic layers were dried over sodium sulfate and concentrated under reduced pressure to get the crude product which was purified by column chromatography using EtOAc and heptane as eluent to afford the desired product as beige solid (0.270 g) (80.27% yield). HPLC/MS (Method 1): Rt: 2.23 min; m/z=677.3 (M+1)⁺. ¹H NMR (300 MHz, DMSO) δ 9.90 (s, 1H), 8.48 (s, 1H), 8.06 (d, J=8.5 Hz, 1H), 7.75 (d, J=8.9 Hz, 2H), 7.66 (d, J=8.8 Hz, 2H), 7.47-7.33 (m, 3H), 7.27 (d, J=8.3 Hz, 1H), 7.06 (s, 1H), 4.18 (s, 1H), 4.10 (s, 1H), 3.90 (t, J=6.4 Hz, 2H), 3.20 (t, J=6.5 Hz, 2H), 2.66 (p, J=6.8 Hz, 1H), 2.31 (s, 3H), 2.16 (s, 3H), 1.13 (dd, J=21.7, 6.8 Hz, 6H).

### Example C-54

### Synthesis of 1-[(E)-[4-[5-chloro-3-methyl-4-[1-oxo-6-(trifluoromethoxy)-3,4-dihydroisoquinolin-2-yl]pyrazol-1-yl]phenyl]methyleneamino]-3-(2-isopropyl-5-methyl-phenyl)thiourea

### Step-1: Synthesis of 4-[3-methyl-4-[1-oxo-6-(trifluoromethoxy)-3,4-dihydroisoquinolin-2-yl]pyrazol-1-yl]benzaldehyde

To a solution of 2-(3-methyl-1H-pyrazol-4-yl)-6-(trifluoromethoxy)-3,4-dihydroisoquinolin-1-one (0.700 g, 2.25 mmol) in 1,2-dichloroethane (7 mL) were added (4-formylphenyl) boronic acid (0.506 g, 3.37 mmol) and copper acetate (0.817 g, 4.50 mmol) at ambient temperature. Then to a reaction mass were added 2,2-bipyridyl (0.703 g, 4.50 mmol) and sodium carbonate (0.477 g, 4.50 mmol) at ambient temperature. The reaction mixture was stirred to 70° C. for 18 h. The progress of the reaction was monitored by TLC. The reaction mixture was diluted with water (25 mL) and was extracted with EtOAc (30 mL×2). The combined organic layers were dried over sodium sulfate and concentrated under reduced pressure to get the crude product which was purified by column chromatography using EtOAc and heptane as eluent to afford the desired product as beige solid (0.360 g) (25.82% yield). HPLC/MS (Method 1): Rt: 2.10 min; m/z=416.3 (M+1)⁺.

### Step-2: Synthesis of 4-[5-chloro-3-methyl-4-[1-oxo-6-(trifluoromethoxy)-3,4-dihydroisoquinolin-2-yl]pyrazol-1-yl]benzaldehyde

To a solution of 4-[3-methyl-4-[1-oxo-6-(trifluoromethoxy)-3,4-dihydroisoquinolin-2-yl]pyrazol-1-yl]benzaldehyde (0.360 g, 0.870 mmol) in chloroform (4 mL) was added palau chlor (0.223 g, 1.07 mmol) at ambient temperature. The reaction mixture was stirred at ambient temperature for 19 h. The progress of the reaction was monitored by TLC. The reaction mixture was diluted with water (15 mL) and was extracted with EtOAc (20 mL×2). The combined organic layers were dried over sodium sulfate and concentrated under reduced pressure to get the crude product which was purified by column chromatography using EtOAc and heptane as eluent to afford the desired product as beige solid (0.330 g) (51.63% yield). HPLC/MS (Method 1): Rt: 2.15 min; m/z=450.1 (M+1)⁺.

### Step-3: Synthesis of 1-[(E)-[4-[5-chloro-3-methyl-4-[1-oxo-6-(trifluoromethoxy)-3,4-dihydroisoquinolin-2-yl]pyrazol-1-yl]phenyl]methyleneamino]-3-(2-isopropyl-5-methyl-phenyl)thiourea (C-54)

To a stirred solution of 4-[5-chloro-3-methyl-4-[1-oxo-6-(trifluoromethoxy)-3,4-dihydroisoquinolin-2-yl]pyrazol-1-yl]benzaldehyde (0.330 g, 0.73 mmol) in acetic acid (4 mL) was added 1-amino-3-(2-isopropyl-5-methyl-phenyl)thiourea (0.164 g, 0.73 mmol) at 0° C. The reaction mass was stirred for 3 h at ambient temperature, and the progress of the reaction was monitored by TLC analysis. After completion of reaction, reaction mixture was quenched in water (15 mL). The precipitated product was filtered through a filter paper and dried under reduced pressure to get the title compound as a solid 0.450 g (93.62% yield). LC/MS (Method 1): Rt: 2.40 min; m/z=655.3 (M+)+; ¹H NMR (300 MHz, DMSO) δ 11.84 (s, 1H), 10.03 (s, 1H), 8.20 (s, 1H), 8.08 (t, J=8.3 Hz, 3H), 7.65 (d, J=8.4 Hz, 2H), 7.47 (s, 1H), 7.40 (d, J=8.7 Hz, 1H), 7.24 (d, J=7.9 Hz, 1H), 7.11 (d, J=8.0 Hz, 1H), 7.01 (s, 1H), 3.89 (t, J=6.6 Hz, 2H), 3.23 (q, J=6.7 Hz, 2H), 3.08 (p, J=6.9 Hz, 1H), 2.29 (s, 3H), 2.20 (s, 3H), 1.17 (d, J=6.8 Hz, 6H).

### Example C-55

### Synthesis of (2Z)-2-[(E)-[4-[5-chloro-3-methyl-4-[1-oxo-6-(trifluoromethoxy)-3,4-dihydroisoquinolin-2-yl]pyrazol-1-yl]phenyl]methylenehydrazono]-3-(2-isopropyl-5-methyl-phenyl)thiazolidin-4-one (C-55)

To a stirred solution of 1-[(E)-[4-[5-chloro-3-methyl-4-[1-oxo-6-(trifluoromethoxy)-3,4-dihydroisoquinolin-2-yl]pyrazol-1-yl]phenyl]methyleneamino]-3-(2-isopropyl-5-methyl-phenyl)thiourea (0.330 g, 0.50 mmol) in ethanol (7 mL) were added sodium acetate (0.083 g, 1.01 mmol) and methyl bromo acetate (0.116 g, 0.760 mmol) at ambient temperature. Then reaction mass was stirred at ambient temperature for 19 h and monitored by TLC analysis. After completion of reaction, reaction mass was diluted with water (20 mL) and was extracted with EtOAc (20 mL×2). The combined organic layers were dried over sodium sulfate and concentrated under reduced pressure to get the crude product which was purified by prep. HPLC using ACN and water as eluent to afford the desired product as beige solid 0.125 g (35.69% yield). LC/MS (Method 1): Rt: 2.43 min; m/z=695.2 (M+)+; ¹H NMR (300 MHz, DMSO) δ 8.42 (s, 1H), 8.07 (d, J=8.6 Hz, 1H), 7.91 (d, J=8.5 Hz, 2H), 7.72 (d, J=8.4 Hz, 2H), 7.50-7.44 (m, 1H), 7.44-7.34 (m, 2H), 7.28 (dd, J=8.1, 1.8 Hz, 1H), 7.15-7.02 (m, 1H), 4.26 (d, J=17.4 Hz, 1H), 4.14 (d, J=17.4 Hz, 1H), 3.89 (t, J=6.5 Hz, 2H), 3.23 (q, J=6.7 Hz, 2H), 2.74 (p, J=6.8 Hz, 1H), 2.32 (s, 3H), 2.20 (s, 3H), 1.13 (dd, J=9.1, 6.8 Hz, 6H).

All other examples enlisted in the table C are synthesized analogous to the methods mentioned in either general procedure or experimental procedure mentioned above. All the compounds listed in the following are preferred embodiments of the present invention.

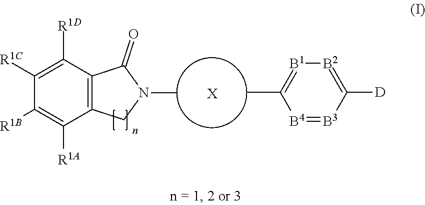

TABLE C

| No | 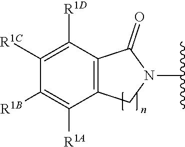 | 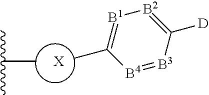 |
|---|---|---|
| C-1 | 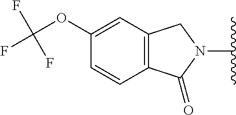 [SMILES: *N1Cc2cc(OC(F)(F)F)ccc2C1=O] | 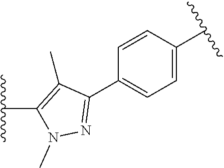 [SMILES: *c1ccc(-c2nn(C)c(*)c2C)cc1] |
| C-2 | 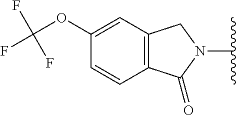 [SMILES: *N1Cc2cc(OC(F)(F)F)ccc2C1=O] | 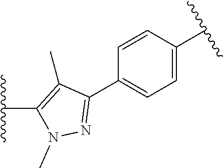 [SMILES: *c1ccc(-c2nn(C)c(*)c2C)cc1] |
| C-3 | 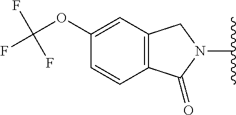 [SMILES: *N1Cc2cc(OC(F)(F)F)ccc2C1=O] | 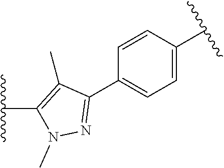 [SMILES: *c1ccc(-c2nn(C)c(*)c2C)cc1] |
| C-4 | 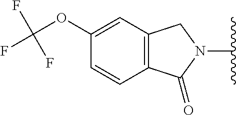 [SMILES: *N1Cc2cc(OC(F)(F)F)ccc2C1=O] | 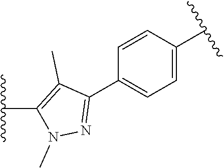 [SMILES: *c1ccc(-c2nn(C)c(*)c2C)cc1] |
| C-5 | 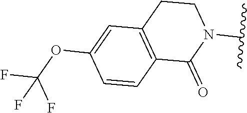 [SMILES: *N1CCc2cc(OC(F)(F)F)ccc2C1=O] | 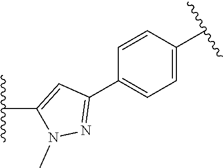 [SMILES: *c1ccc(-c2cc(*)n(C)n2)cc1] |
| C-6 | 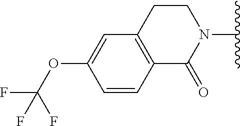 [SMILES: *N1CCc2cc(OC(F)(F)F)ccc2C1=O] | 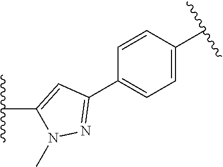 [SMILES: *c1ccc(-c2cc(*)n(C)n2)cc1] |
| C-7 | 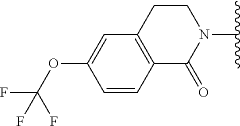 [SMILES: *N1CCc2cc(OC(F)(F)F)ccc2C1=O] | 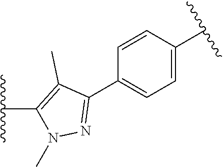 [SMILES: *c1ccc(-c2nn(C)c(*)c2C)cc1] |
| C-8 | 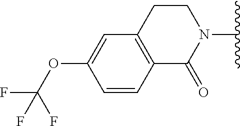 [SMILES: *N1CCc2cc(OC(F)(F)F)ccc2C1=O] | 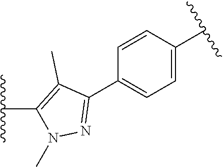 [SMILES: *c1ccc(-c2nn(C)c(*)c2C)cc1] |
| C-9 | 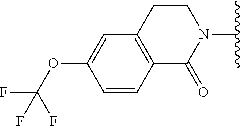 [SMILES: *N1CCc2cc(OC(F)(F)F)ccc2C1=O] | 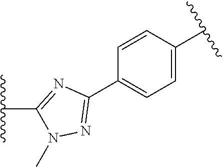 [SMILES: *c1ccc(-c2nc(*)n(C)n2)cc1] |
| C-10 | 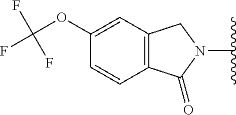 [SMILES: *N1Cc2cc(OC(F)(F)F)ccc2C1=O] | 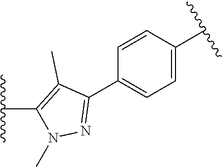 [SMILES: *c1ccc(-c2nn(C)c(*)c2C)cc1] |
| C-11 | 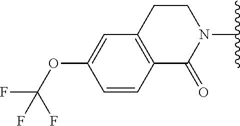 [SMILES: *N1CCc2cc(OC(F)(F)F)ccc2C1=O] | 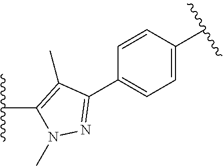 [SMILES: *c1ccc(-c2nn(C)c(*)c2C)cc1] |
| C-12 | 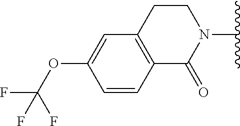 [SMILES: *N1CCc2cc(OC(F)(F)F)ccc2C1=O] | 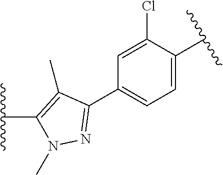 [SMILES: *c1ccc(-c2nn(C)c(*)c2C)cc1Cl] |
| C-13 | 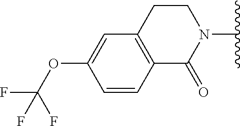 [SMILES: *N1CCc2cc(OC(F)(F)F)ccc2C1=O] | 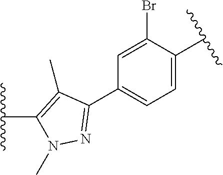 [SMILES: *c1ccc(-c2nn(C)c(*)c2C)cc1Br] |
| C-14 | 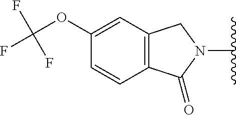 [SMILES: *N1Cc2cc(OC(F)(F)F)ccc2C1=O] | 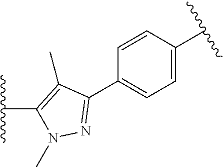 [SMILES: *c1ccc(-c2nn(C)c(*)c2C)cc1] |
| C-15 | 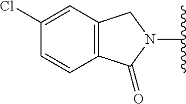 [SMILES: *N1Cc2cc(Cl)ccc2C1=O] | 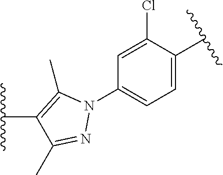 [SMILES: *c1ccc(-n2nc(C)c(*)c2C)cc1Cl] |
| C-16 | 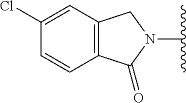 [SMILES: *N1Cc2cc(Cl)ccc2C1=O] | 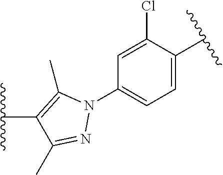 [SMILES: *c1ccc(-n2nc(C)c(*)c2C)cc1Cl] |
| C-17 | 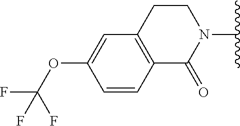 [SMILES: *N1CCc2cc(OC(F)(F)F)ccc2C1=O] | 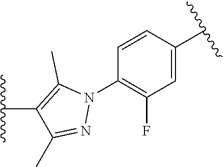 [SMILES: *c1ccc(-n2nc(C)c(*)c2C)c(F)c1] |
| C-18 | 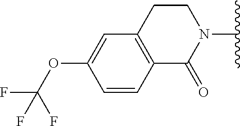 [SMILES: *N1CCc2cc(OC(F)(F)F)ccc2C1=O] | 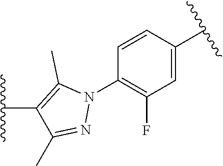 [SMILES: *c1ccc(-n2nc(C)c(*)c2C)c(F)c1] |
| C-19 | 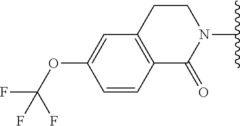 [SMILES: *N1CCc2cc(OC(F)(F)F)ccc2C1=O] | 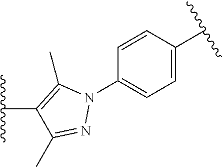 [SMILES: *c1ccc(-n2nc(C)c(*)c2C)cc1] |
| C-20 | 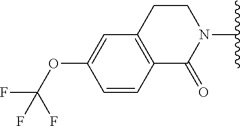 [SMILES: *N1CCc2cc(OC(F)(F)F)ccc2C1=O] | 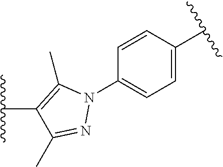 [SMILES: *c1ccc(-n2nc(C)c(*)c2C)cc1] |
| C-21 | 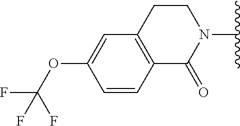 [SMILES: *N1CCc2cc(OC(F)(F)F)ccc2C1=O] | 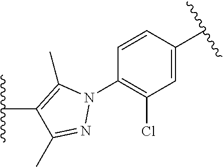 [SMILES: *c1ccc(-n2nc(C)c(*)c2C)c(Cl)c1] |
| C-22 | 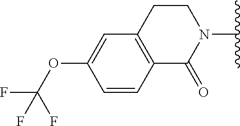 [SMILES: *N1CCc2cc(OC(F)(F)F)ccc2C1=O] | 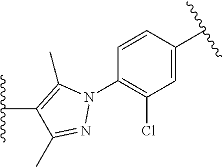 [SMILES: *c1ccc(-n2nc(C)c(*)c2C)c(Cl)c1] |
| C-23 | 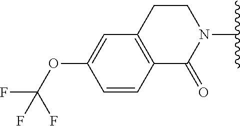 [SMILES: *N1CCc2cc(OC(F)(F)F)ccc2C1=O] | 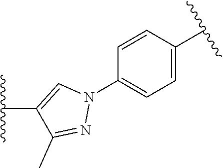 [SMILES: *c1ccc(-n2cc(*)c(C)n2)cc1] |
| C-24 | 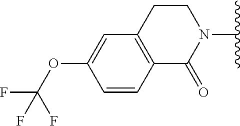 [SMILES: *N1CCc2cc(OC(F)(F)F)ccc2C1=O] | 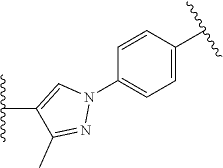 [SMILES: *c1ccc(-n2cc(*)c(C)n2)cc1] |
| C-25 | 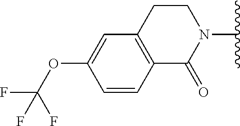 [SMILES: *N1CCc2cc(OC(F)(F)F)ccc2C1=O] | 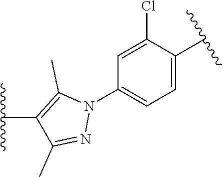 [SMILES: *c1ccc(-n2nc(C)c(*)c2C)cc1Cl] |
| C-26 | 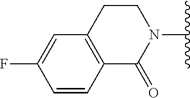 [SMILES: *N1CCc2cc(F)ccc2C1=O] | 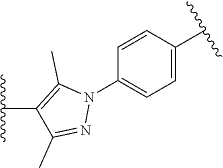 [SMILES: *c1ccc(-n2nc(C)c(*)c2C)cc1] |
| C-27 | 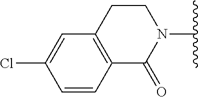 [SMILES: *N1CCc2cc(Cl)ccc2C1=O] | 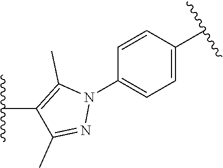 [SMILES: *c1ccc(-n2nc(C)c(*)c2C)cc1] |
| C-28 | 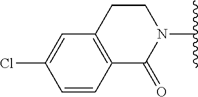 [SMILES: *N1CCc2cc(Cl)ccc2C1=O] | 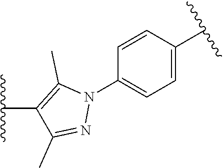 [SMILES: *c1ccc(-n2nc(C)c(*)c2C)cc1] |
| C-29 | 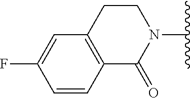 [SMILES: *N1CCc2cc(F)ccc2C1=O] | 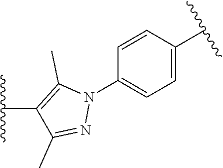 [SMILES: *c1ccc(-n2nc(C)c(*)c2C)cc1] |
| C-30 | 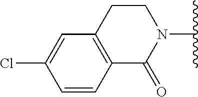 [SMILES: *N1CCc2cc(Cl)ccc2C1=O] | 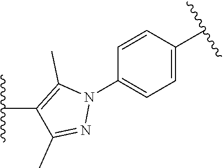 [SMILES: *c1ccc(-n2nc(C)c(*)c2C)cc1] |
| C-31 | 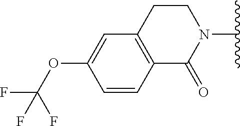 [SMILES: *N1CCc2cc(OC(F)(F)F)ccc2C1=O] | 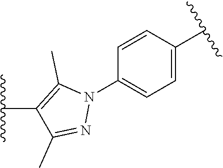 [SMILES: *c1ccc(-n2nc(C)c(*)c2C)cc1] |
| C-32 | 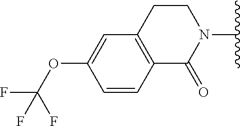 [SMILES: *N1CCc2cc(OC(F)(F)F)ccc2C1=O] | 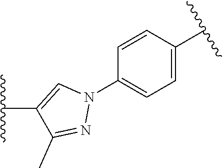 [SMILES: *c1ccc(-n2cc(*)c(C)n2)cc1] |
| C-33 | 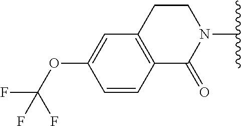 [SMILES: *N1CCc2cc(OC(F)(F)F)ccc2C1=O] | 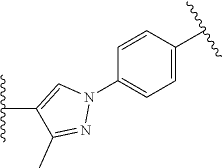 [SMILES: *c1ccc(-n2cc(*)c(C)n2)cc1] |
| C-34 | 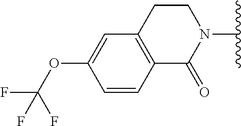 [SMILES: *N1CCc2cc(OC(F)(F)F)ccc2C1=O] | 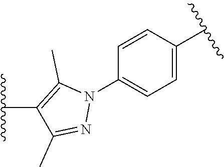 [SMILES: *c1ccc(-n2nc(C)c(*)c2C)cc1] |
| C-35 | 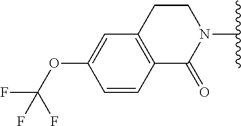 [SMILES: *N1CCc2cc(OC(F)(F)F)ccc2C1=O] | 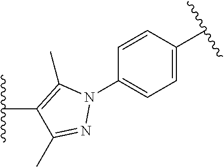 [SMILES: *c1ccc(-n2nc(C)c(*)c2C)cc1] |
| C-36 | 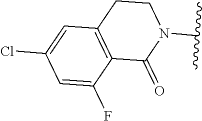 [SMILES: *N1CCc2cc(Cl)cc(F)c2C1=O] | 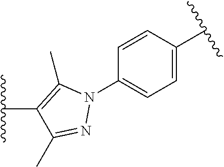 [SMILES: *c1ccc(-n2nc(C)c(*)c2C)cc1] |
| C-37 | 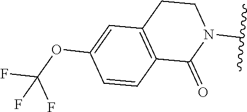 [SMILES: *N1CCc2cc(OC(F)(F)F)ccc2C1=O] | 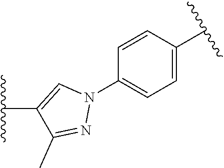 [SMILES: *c1ccc(-n2cc(*)c(C)n2)cc1] |
| C-38 | 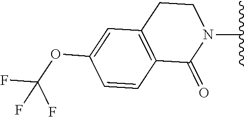 [SMILES: *N1CCc2cc(OC(F)(F)F)ccc2C1=O] | 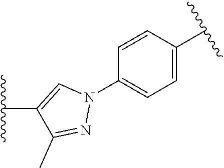 [SMILES: *c1ccc(-n2cc(*)c(C)n2)cc1] |
| C-39 | 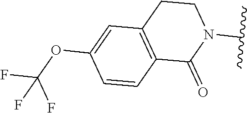 [SMILES: *N1CCc2cc(OC(F)(F)F)ccc2C1=O] | 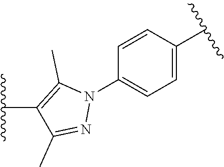 [SMILES: *c1ccc(-n2nc(C)c(*)c2C)cc1] |
| C-40 | 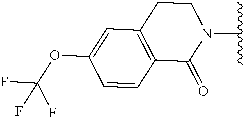 [SMILES: *N1CCc2cc(OC(F)(F)F)ccc2C1=O] | 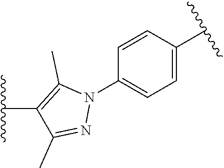 [SMILES: *c1ccc(-n2nc(C)c(*)c2C)cc1] |
| C-41 | 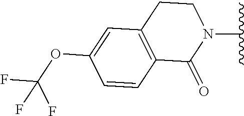 [SMILES: *N1CCc2cc(OC(F)(F)F)ccc2C1=O] | 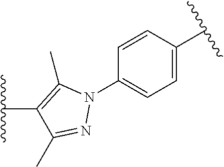 [SMILES: *c1ccc(-n2nc(C)c(*)c2C)cc1] |
| C-42 | 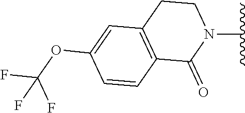 [SMILES: *N1CCc2cc(OC(F)(F)F)ccc2C1=O] | 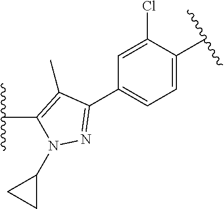 [SMILES: *c1ccc(-c2nn(C3CC3)c(*)c2C)cc1Cl] |
| C-43 | 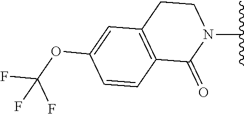 [SMILES: *N1CCc2cc(OC(F)(F)F)ccc2C1=O] | 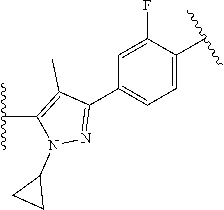 [SMILES: *c1ccc(-c2nn(C3CC3)c(*)c2C)cc1F] |
| C-44 | 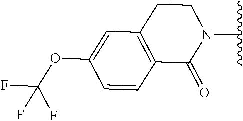 [SMILES: *N1CCc2cc(OC(F)(F)F)ccc2C1=O] | 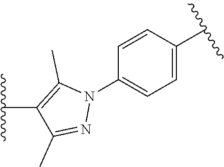 [SMILES: *c1ccc(-n2nc(C)c(*)c2C)cc1] |
| C-45 | 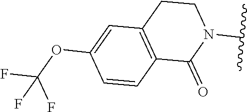 [SMILES: *N1CCc2cc(OC(F)(F)F)ccc2C1=O] | 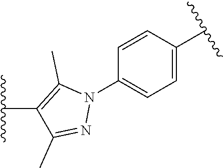 [SMILES: *c1ccc(-n2nc(C)c(*)c2C)cc1] |
| C-46 | 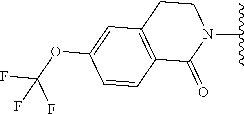 [SMILES: *N1CCc2cc(OC(F)(F)F)ccc2C1=O] | 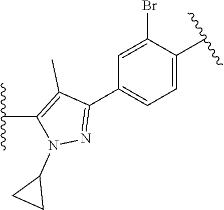 [SMILES: *c1ccc(-c2nn(C3CC3)c(*)c2C)cc1Br] |
| C-47 | 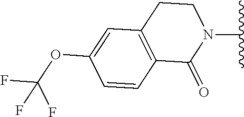 [SMILES: *N1CCc2cc(OC(F)(F)F)ccc2C1=O] | 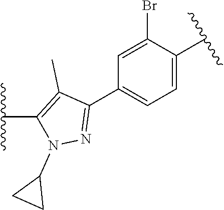 [SMILES: *c1ccc(-c2nn(C3CC3)c(*)c2C)cc1Br] |
| C-48 | 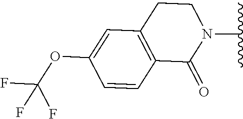 [SMILES: *N1CCc2cc(OC(F)(F)F)ccc2C1=O] | 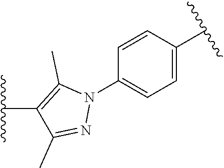 [SMILES: *c1ccc(-n2nc(C)c(*)c2C)cc1] |
| C-49 | 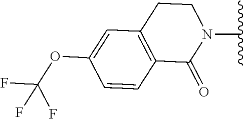 [SMILES: *N1CCc2cc(OC(F)(F)F)ccc2C1=O] | 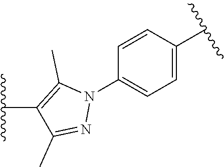 [SMILES: *c1ccc(-n2nc(C)c(*)c2C)cc1] |
| C-50 | 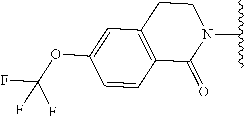 [SMILES: *N1CCc2cc(OC(F)(F)F)ccc2C1=O] | 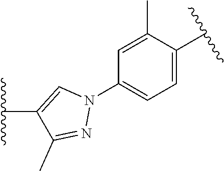 [SMILES: *c1ccc(-n2cc(*)c(C)n2)cc1C] |
| C-51 | 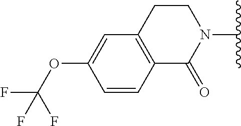 [SMILES: *N1CCc2cc(OC(F)(F)F)ccc2C1=O] | 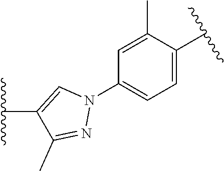 [SMILES: *c1ccc(-n2cc(*)c(C)n2)cc1C] |
| C-52 | 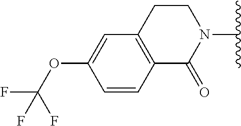 [SMILES: *N1CCc2cc(OC(F)(F)F)ccc2C1=O] | 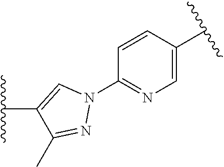 [SMILES: *c1ccc(-n2cc(*)c(C)n2)nc1] |
| C-53 | 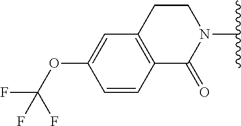 [SMILES: *N1CCc2cc(OC(F)(F)F)ccc2C1=O] | 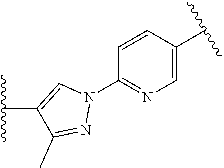 [SMILES: *c1ccc(-n2cc(*)c(C)n2)nc1] |
| C-54 | 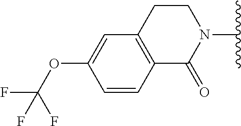 [SMILES: *N1CCc2cc(OC(F)(F)F)ccc2C1=O] | 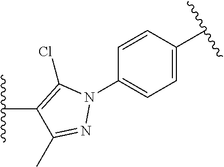 [SMILES: *c1ccc(-n2nc(C)c(*)c2Cl)cc1] |
| C-55 | 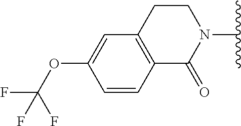 [SMILES: *N1CCc2cc(OC(F)(F)F)ccc2C1=O] | 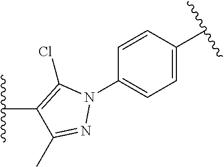 [SMILES: *c1ccc(-n2nc(C)c(*)c2Cl)cc1] |
| C-56 | 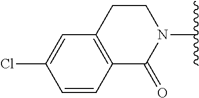 [SMILES: *N1CCc2cc(Cl)ccc2C1=O] | 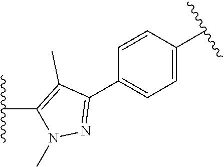 [SMILES: *c1ccc(-c2nn(C)c(*)c2C)cc1] |
| C-57 | 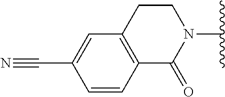 [SMILES: *N1CCc2cc(C#N)ccc2C1=O] | 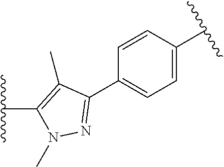 [SMILES: *c1ccc(-c2nn(C)c(*)c2C)cc1] |
| C-58 | 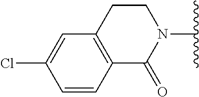 [SMILES: *N1CCc2cc(Cl)ccc2C1=O] | 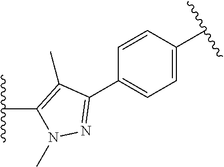 [SMILES: *c1ccc(-c2nn(C)c(*)c2C)cc1] |
| C-59 | 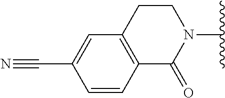 [SMILES: *N1CCc2cc(C#N)ccc2C1=O] | 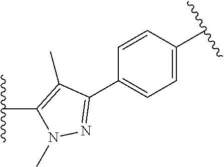 [SMILES: *c1ccc(-c2nn(C)c(*)c2C)cc1] |
| C-60 | 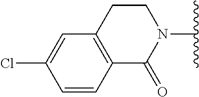 [SMILES: *N1CCc2cc(Cl)ccc2C1=O] | 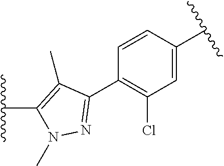 [SMILES: *c1ccc(-c2nn(C)c(*)c2C)c(Cl)c1] |
| C-61 | 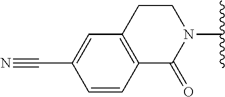 [SMILES: *N1CCc2cc(C#N)ccc2C1=O] | 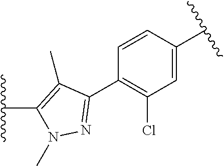 [SMILES: *c1ccc(-c2nn(C)c(*)c2C)c(Cl)c1] |
| C-62 | 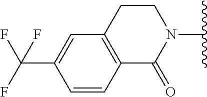 [SMILES: *N1CCc2cc(C(F)(F)F)ccc2C1=O] | 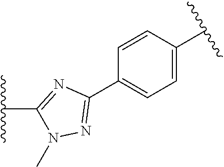 [SMILES: *c1ccc(-c2nc(*)n(C)n2)cc1] |
| C-63 | 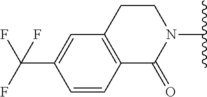 [SMILES: *N1CCc2cc(C(F)(F)F)ccc2C1=O] | 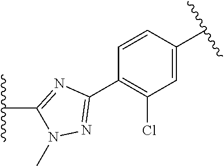 [SMILES: *c1ccc(-c2nc(*)n(C)n2)c(Cl)c1] |
| C-64 | 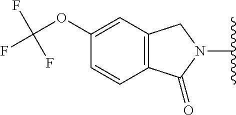 [SMILES: *N1Cc2cc(OC(F)(F)F)ccc2C1=O] | 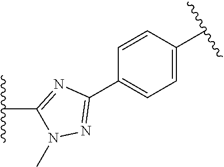 [SMILES: *c1ccc(-c2nc(*)n(C)n2)cc1] |
| C-65 | 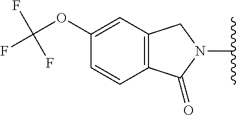 [SMILES: *N1Cc2cc(OC(F)(F)F)ccc2C1=O] | 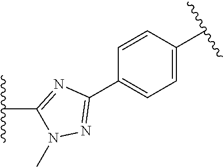 [SMILES: *c1ccc(-c2nc(*)n(C)n2)cc1] |
| C-66 | 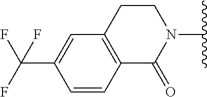 [SMILES: *N1CCc2cc(C(F)(F)F)ccc2C1=O] | 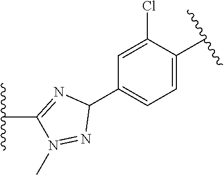 |

| | | | Rt |
|---|---|---|---|
| No. | D | HPLC/MS | min |
| C-1 | 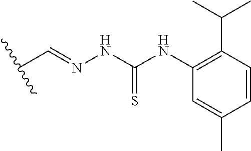 [SMILES: */C=N/NC(=S)Nc1cc(C)ccc1C(C)C] | 21 (Method 2) | 1.41 |
| C-2 | 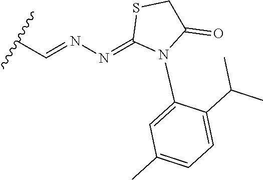 [SMILES: */C=N/N=C1\SCC(=O)N1c1cc(C)ccc1C(C)C] | 661 (Method 2) | 1.44 |
| C-3 | 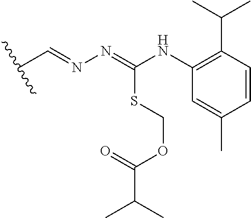 [SMILES: */C=N/N=C(/Nc1cc(C)ccc1C(C)C)SCOC(=O)C(C)C] | 721.4 (Method 2) | 1.48 |
| C-4 | 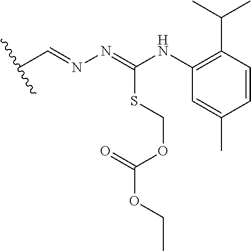 [SMILES: */C=N/N=C(/Nc1cc(C)ccc1C(C)C)SCOC(=O)OCC] | 723.5 (Method 2) | 1.46 |
| C-5 | 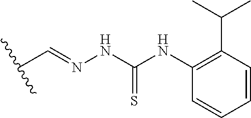 [SMILES: */C=N/NC(=S)Nc1ccccc1C(C)C] | 607 (Method 1) | 2.12 |
| C-6 | 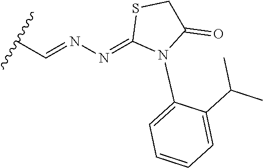 [SMILES: */C=N/N=C1\SCC(=O)N1c1ccccc1C(C)C] | 647 (Method 1) | 2.16 |
| C-7 | 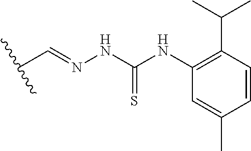 [SMILES: */C=N/NC(=S)Nc1cc(C)ccc1C(C)C] | 635 (Method 1) | 2.34 |
| C-8 | 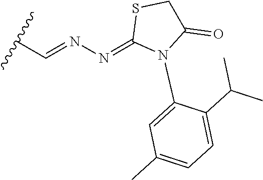 [SMILES: */C=N/N=C1\SCC(=O)N1c1cc(C)ccc1C(C)C] | 675 (Method 1) | 2.38 |
| C-9 | 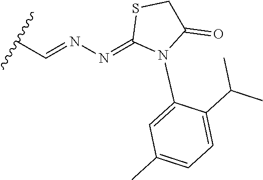 [SMILES: */C=N/N=C1\SCC(=O)N1c1cc(C)ccc1C(C)C] | 662 (Method 1) | 2.37 |
| C-10 | 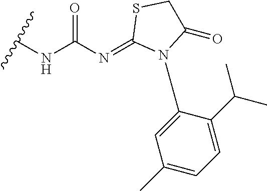 [SMILES: *NC(=O)/N=C1\SCC(=O)N1c1cc(C)ccc1C(C)C] | 677 (Method 2) | 1.36 |
| C-11 | 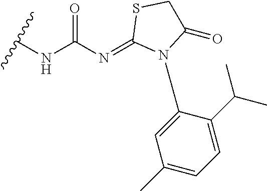 [SMILES: *NC(=O)/N=C1\SCC(=O)N1c1cc(C)ccc1C(C)C] | 691 (Method 1) | 2.24 |
| C-12 | 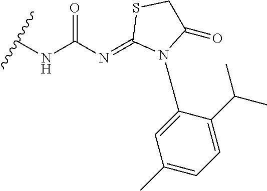 [SMILES: *NC(=O)/N=C1\SCC(=O)N1c1cc(C)ccc1C(C)C] | 725 (Method 1) | 2.36 |
| C-13 | 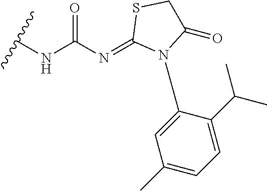 [SMILES: *NC(=O)/N=C1\SCC(=O)N1c1cc(C)ccc1C(C)C] | 771 (Method 1) | 2.36 |
| C-14 | 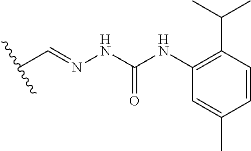 [SMILES: */C=N/NC(=O)Nc1cc(C)ccc1C(C)C] | 605.3 (Method 2) | 1.37 |
| C-15 | 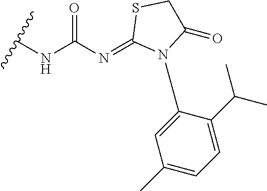 [SMILES: *NC(=O)/N=C1\SCC(=O)N1c1cc(C)ccc1C(C)C] | 661.0 (Method 2) | 1.42 |
| C-16 | 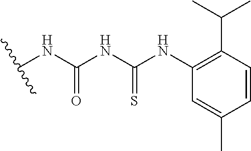 [SMILES: *NC(=O)NC(=S)Nc1cc(C)ccc1C(C)C] | 621.0 (Method 2) | 1.29 |
| C-17 | 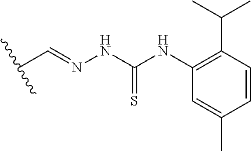 [SMILES: */C=N/NC(=S)Nc1cc(C)ccc1C(C)C] | 653.3 (Method 1) | 2.32 |
| C-18 | 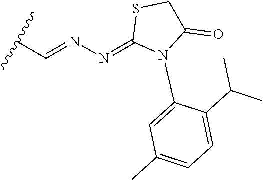 [SMILES: */C=N/N=C1\SCC(=O)N1c1cc(C)ccc1C(C)C] | 693.3 (Method 1) | 2.36 |
| C-19 | 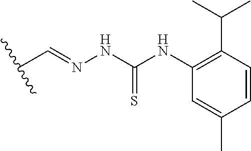 [SMILES: */C=N/NC(=S)Nc1cc(C)ccc1C(C)C] | 635.4 (Method 1) | 2.32 |
| C-20 | 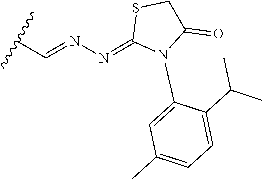 [SMILES: */C=N/N=C1\SCC(=O)N1c1cc(C)ccc1C(C)C] | 675.3 (Method 1) | 2.35 |
| C-21 | 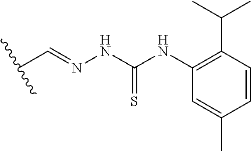 [SMILES: */C=N/NC(=S)Nc1cc(C)ccc1C(C)C] | 669.3 (Method 1) | 2.33 |
| C-22 | 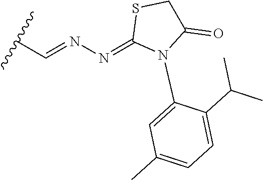 [SMILES: */C=N/N=C1\SCC(=O)N1c1cc(C)ccc1C(C)C] | 709.3 (Method 1) | 2.37 |
| C-23 | 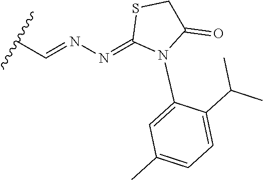 [SMILES: */C=N/N=C1\SCC(=O)N1c1cc(C)ccc1C(C)C] | 661.4 (Method 1) | 2.36 |
| C-24 | 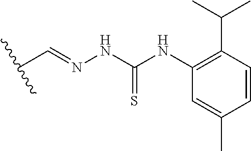 [SMILES: */C=N/NC(=S)Nc1cc(C)ccc1C(C)C] | 619.3 (Method 1) | 2.32 |
| C-25 | 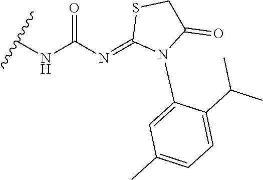 [SMILES: *NC(=O)/N=C1\SCC(=O)N1c1cc(C)ccc1C(C)C] | 725.3 (Method 1) | 2.31 |
| C-26 | 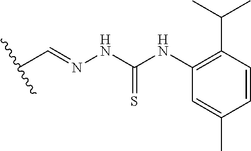 [SMILES: */C=N/NC(=S)Nc1cc(C)ccc1C(C)C] | 569.2 (Method 2) | 1.34 |
| C-27 | 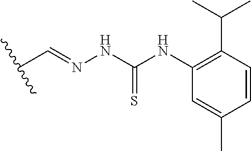 [SMILES: */C=N/NC(=S)Nc1cc(C)ccc1C(C)C] | 585.2 (Method 2) | 1.39 |
| C-28 | 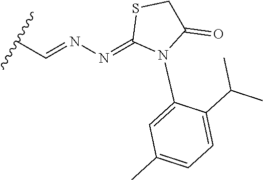 [SMILES: */C=N/N=C1\SCC(=O)N1c1cc(C)ccc1C(C)C] | 625.2 (Method 2) | 1.43 |
| C-29 | 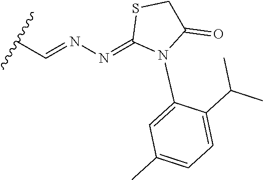 [SMILES: */C=N/N=C1\SCC(=O)N1c1cc(C)ccc1C(C)C] | 609.3 (Method 2) | 1.37 |
| C-30 | 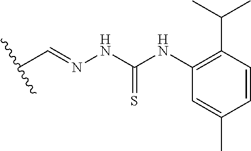 [SMILES: */C=N/NC(=S)Nc1cc(C)ccc1C(C)C] | 603 (Method 2) | 1.37 |
| C-31 | 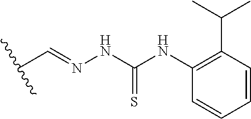 [SMILES: */C=N/NC(=S)Nc1ccccc1C(C)C] | 621.3 (Method 1) | 2.25 |
| C-32 | 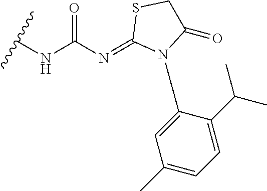 [SMILES: *NC(=O)/N=C1\SCC(=O)N1c1cc(C)ccc1C(C)C] | 677.3 (Method 1) | 2.23 |
| C-33 | 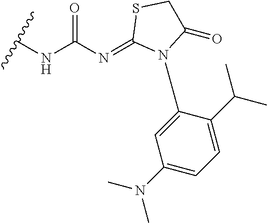 [SMILES: *NC(=O)/N=C1\SCC(=O)N1c1cc(N(C)C)ccc1C(C)C] | 706.2 (Method 1) | 2.25 |
| C-34 | 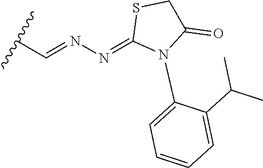 [SMILES: */C=N/N=C1\SCC(=O)N1c1ccccc1C(C)C] | 661.4 (Method 1) | 2.30 |
| C-35 | 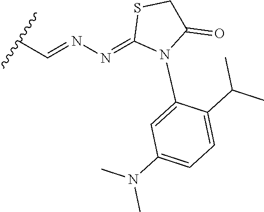 [SMILES: */C=N/N=C1\SCC(=O)N1c1cc(N(C)C)ccc1C(C)C] | 704.4 (Method 1) | 2.34 |
| C-36 | 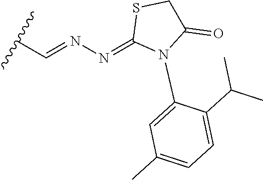 [SMILES: */C=N/N=C1\SCC(=O)N1c1cc(C)ccc1C(C)C] | 643.1 (Method 2) | 1.40 |
| C-37 | 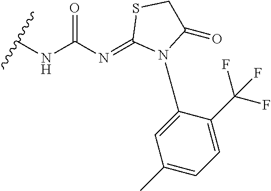 [SMILES: *NC(=O)/N=C1\SCC(=O)N1c1cc(C)ccc1C(F)(F)F] | 703.3 (Method 1) | 2.19 |
| C-38 | 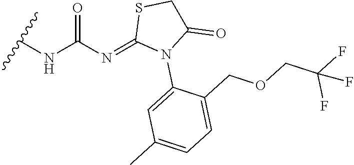 [SMILES: *NC(=O)/N=C1\SCC(=O)N1c1cc(C)ccc1COCC(F)(F)F] | 747.3 (Method 1) | 2.20 |
| C-39 | 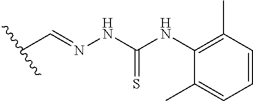 [SMILES: */C=N/NC(=S)Nc1c(C)cccc1C] | 607.4 (Method 1) | 2.19 |
| C-40 | 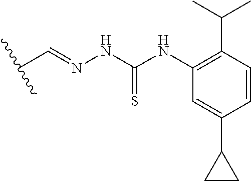 [SMILES: */C=N/NC(=S)Nc1cc(C2CC2)ccc1C(C)C] | 661.4 (Method 1) | 2.36 |
| C-41 | 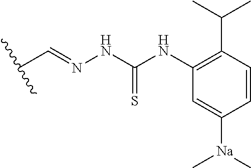 | 664.4 (Method 1) | 2.26 |
| C-42 | 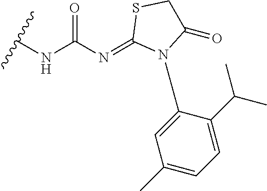 [SMILES: *NC(=O)/N=C1\SCC(=O)N1c1cc(C)ccc1C(C)C] | 751.1 (Method 2) | 1.23 |
| C-43 | 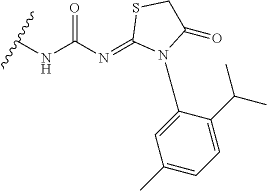 [SMILES: *NC(=O)/N=C1\SCC(=O)N1c1cc(C)ccc1C(C)C] | 735.1 (Method 2) | 1.20 |
| C-44 | 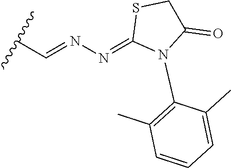 [SMILES: */C=N/N=C1\SCC(=O)N1c1c(C)cccc1C] | 647.3 (Method 1) | 2.25 |
| C-45 | 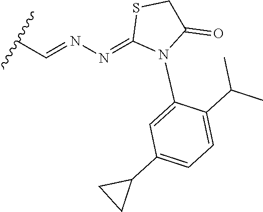 [SMILES: */C=N/N=C1\SCC(=O)N1c1cc(C2CC2)ccc1C(C)C] | 701.4 (Method 1) | 2.37 |
| C-46 | 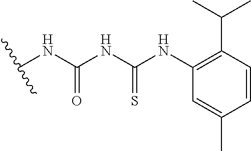 [SMILES: *NC(=O)NC(=S)Nc1cc(C)ccc1C(C)C] | 757.0 (Method 2) | 1.31 |
| C-47 | 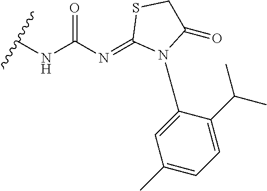 [SMILES: *NC(=O)/N=C1\SCC(=O)N1c1cc(C)ccc1C(C)C] | 795.0 (Method 1) | 4.42 |
| C-48 | 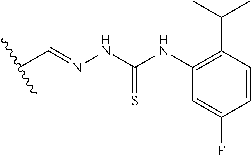 [SMILES: */C=N/NC(=S)Nc1cc(F)ccc1C(C)C] | 639.4 (Method 1) | 2.29 |
| C-49 | 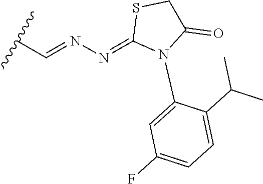 [SMILES: */C=N/N=C1\SCC(=O)N1c1cc(F)ccc1C(C)C] | 679.3 (Method 1) | 2.32 |
| C-50 | 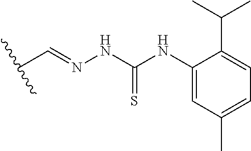 [SMILES: */C=N/NC(=S)Nc1cc(C)ccc1C(C)C] | 635.4 (Method 1) | 2.41 |
| C-51 | 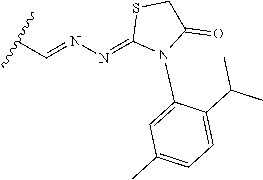 [SMILES: */C=N/N=C1\SCC(=O)N1c1cc(C)ccc1C(C)C] | 675.4 (Method 1) | 2.45 |
| C-52 | 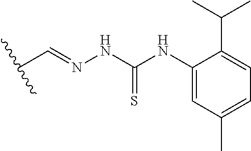 [SMILES: */C=N/NC(=S)Nc1cc(C)ccc1C(C)C] | 622.3 (Method 1) | 2.37 |
| C-53 | 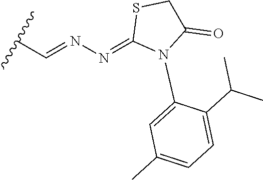 [SMILES: */C=N/N=C1\SCC(=O)N1c1cc(C)ccc1C(C)C] | 662.3 (Method 1) | 2.41 |
| C-54 | 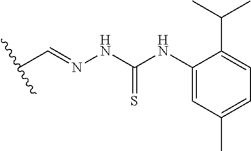 [SMILES: */C=N/NC(=S)Nc1cc(C)ccc1C(C)C] | 655.3 (Method 1) | 2.40 |
| C-55 | 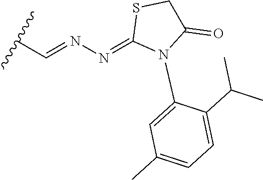 [SMILES: */C=N/N=C1\SCC(=O)N1c1cc(C)ccc1C(C)C] | 695.2 (Method 1) | 2.43 |
| C-56 | 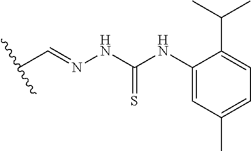 [SMILES: */C=N/NC(=S)Nc1cc(C)ccc1C(C)C] | 585.1 (Method 2) | 1.43 |
| C-57 | 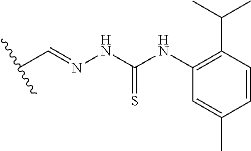 [SMILES: */C=N/NC(=S)Nc1cc(C)ccc1C(C)C] | 576.1 (Method 2) | 1.33 |
| C-58 | 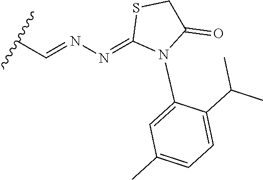 [SMILES: */C=N/N=C1\SCC(=O)N1c1cc(C)ccc1C(C)C] | 625.1 (Method 2) | 1.46 |
| C-59 | 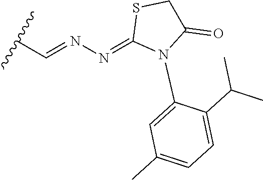 [SMILES: */C=N/N=C1\SCC(=O)N1c1cc(C)ccc1C(C)C] | 616.1 (Method 2) | 1.37 |
| C-60 | 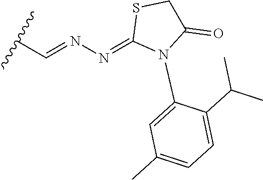 [SMILES: */C=N/N=C1\SCC(=O)N1c1cc(C)ccc1C(C)C] | 659 (Method 2) | 1.49 |
| C-61 | 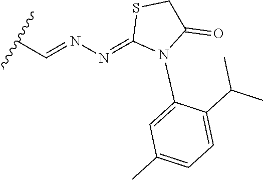 [SMILES: */C=N/N=C1\SCC(=O)N1c1cc(C)ccc1C(C)C] | 650.1 (Method 2) | 1.39 |
| C-62 | 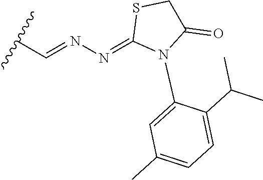 [SMILES: */C=N/N=C1\SCC(=O)N1c1cc(C)ccc1C(C)C] | 646.1 (Method 2) | 1.46 |
| C-63 | 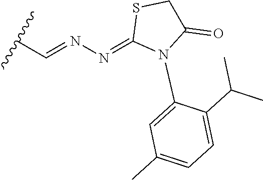 [SMILES: */C=N/N=C1\SCC(=O)N1c1cc(C)ccc1C(C)C] | 680 (Method 2) | 1.48 |
| C-64 | 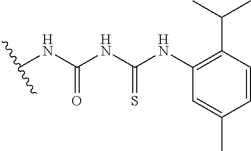 [SMILES: *NC(=O)NC(=S)Nc1cc(C)ccc1C(C)C] | 624.1 (Method 2) | 1.06 |
| C-65 | 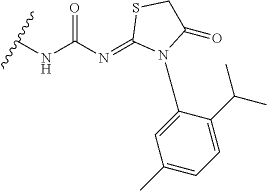 [SMILES: *NC(=O)/N=C1\SCC(=O)N1c1cc(C)ccc1C(C)C] | 664.0 (Method 2) | 1.30 |
| C-66 | 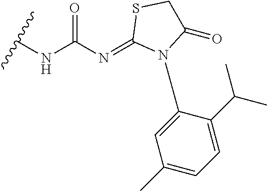 [SMILES: *NC(=O)/N=C1\SCC(=O)N1c1cc(C)ccc1C(C)C] | 696 (Method 2) | 1.45 |

### Biological Examples

### Example B1: Action on Yellow Fever Mosquito (Aedes aegypti)

For evaluating control of yellow fever mosquito (Aedes aegypti) the test unit consisted of 96-well-microtiter plates containing 200 μL of tap water per well and 5-15 freshly hatched A. aegypti larvae.

The active compounds or mixtures were formulated using a solution containing 75% (v/v) water and 25% (v/v) DMSO. Different concentrations of formulated compounds or mixtures were sprayed onto the insect diet at 2.5 μL, using a custom-built micro atomizer, at two replications.

For experimental mixtures in these tests identical volumes of both mixing partners at the desired concentrations respectively, were mixed together.

After application, microtiter plates were incubated at 28±1° C., 80±5% relative humidity for 2 days. Larval mortality was then visually assessed.

In this test, compounds C-1, C-2, C-3, C-4, C-5, C-7, C-8, C-9, C-10, C-11, C-12, C-13, C-14, C-15, C-16, C-17, C-18, C-19, C-20, C-21, C-23, C-24, C-25, C-26, C-28, C-29, C-30, C-32, C-33, C-34, C-35, C-36, C-37, C-38, C-41, C-42, C-43, C-44, C-45, C-46, C-47, C-49, C-50, C-51, C-52, C-53, C-54, C-55, C-56, C-57, C-59, C-60, C-61, C-62, C-63, C-64, C-65, C-66 at 800 ppm showed at least 50% mortality in comparison with untreated controls.

### Example B2: Action on Orchid Thrips (Dichromothrips corbetti)

Dichromothrips corbetti adults used for bioassay were obtained from a colony maintained continuously under laboratory conditions. For testing purposes, the test compound is diluted in a 1:1 mixture of acetone: water (vol: vol), plus Kinetic® HV at a rate of 0.01% v/v.

Thrips potency of each compound was evaluated by using a floral-immersion technique. All petals of individual, intact orchid flowers were dipped into treatment solution and allowed to drying Petri dishes. Treated petals were placed into individual re-sealable plastic along with about 20 adult thrips. All test arenas were held under continuous light and a temperature of about 28° C. for duration of the assay. After 3 days, the numbers of live thrips were counted on each petal. The percent mortality was recorded 72 hours after treatment.

In this test, compounds C-1, C-2, C-3, C-4, C-5, C-6, C-7, C-8, C-9, C-11, C-12, C-13, C-14, C-15, C-16, C-17, C-18, C-19, C-20, C-23, C-24, C-25, C-33, C-34, C-35, C-40, C-41, C-42, C-43, C-45, C-46, C-47, C-49, C-50, C-52, C-53, C-54, C-55, C-62, C-65 at 300 ppm showed at least 75% mortality in comparison with untreated controls.

### Example B3: Action on Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% (v/v) water and 25% (v/v) DMSO. Different concentrations of formulated compounds were sprayed onto the insect diet at 5 μL, using a custom-built micro atomizer, at two replications.

After application, microtiter plates were incubated at about 25±1° C. and about 75±5% relative humidity for 5 days. Egg and larval mortality were then visually assessed.

In this test, compounds C-1, C-2, C-3, C-4, C-5, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66 at 800 ppm showed at least 75% mortality in comparison with untreated controls.

### Example B4: Action on Silverleaf Whitefly (Bemisia argentifolii) (Adults)

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone: 50% water (v/v) into 5 or 10 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, compound C-2, C-5, C-18, C-35, C-38, C-42, C-43, C-46, C-47 at 300 ppm showed at least 75% mortality in comparison with untreated controls.

### Example B5: Action on Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1° C. and about 80±5% relative humidity for 5 days. Egg and larval mortality were then visually assessed.

In this test, compounds C-1, C-2, C-3, C-4, C-5, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, at 800 ppm showed at least 75% mortality in comparison with untreated controls.

### Example B6: Action on Diamond Back Moth (Plutella xylostella)

The active compound is dissolved at the desired concentration in a mixture of 1:1 (v/v) distilled water:acetone. Surfactant (Kinetic® HV) is added at a rate of 0.01% (v/v). The test solution is prepared at the day of use.

Leaves of cabbage were dipped in test solution and air-dried. Treated leaves were placed in petri dishes lined with moist filter paper and inoculated with ten third instar larvae. Mortality was recorded 72 hours after treatment. Feeding damages were also recorded using a scale of 0-100%.

In this test, compounds C-1, C-2, C-3, C-4, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66 at 300 ppm showed at least 75% mortality in comparison with untreated controls.

### Example B7: Action on Southern Armyworm (Spodoptera eridania), Second Instar Larvae

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone: 50% water (v/v) into 10 or 20 mL glass vials. A nonionic surfactant (Kinetic®) was included in the solution at a volume of 0.01% (v/v). The vials were then inserted into an automated electrostatic sprayer equipped with an atomizing nozzle for application to plants/insects.

Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, compounds C-1, C-2, C-3, C-4, C-5, C-6, C-9, C-12, C-13, C-14 at 300 ppm showed at least 75% mortality in comparison with untreated controls.

### Example B8: Action on Diamond Back Moth (Plutella xylostella)

For evaluating control of diamond back moth (Plutella xylostella) the test unit consisted of 96-well-microtiter plates containing an insect diet and 15-25 P. xylostella eggs. The compounds or mixtures were formulated using a solution containing 75% water and 25% DMSO. Different concentrations of formulated compounds or mixtures were sprayed onto the insect diet at 5 μl, using a custom-built micro atomizer, at two replications. For experimental mixtures in these tests identical volumes of both mixing partners at the desired concentrations respectively, were mixed together. After application, microtiter plates were incubated at 28±1° C., 80±5% relative humidity for 5 days. Egg and larval mortality was then visually assessed. In this test, compounds C-3, C-4, C-7, C-9 and C-10 at 800 ppm showed at least 75% mortality in comparison with untreated controls.

1. A compound of the formula I 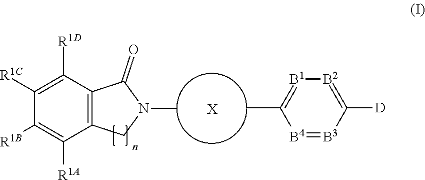
wherein
R^{1A}, R^{1B}, R^{1C}, and R^{1D} are, identical or different, H, halogen, SF₅, OH, CN, C₁-C₆-alkyl, C₁-C₆-alkoxy, C₃-C₆-cycloalkyl, C₃-C₆-cycloalkoxy, wherein the alkyl, alkoxy, cycloalkyl, and cycloalkoxy moieties are unsubstituted or substituted with halogen or CN;
NR⁸R⁹, C═O(NR⁸R⁹), S(═O)ₘ(NR⁸R⁹), or NHS(═O)ₘR⁸;
m is 1 or 2;
n is 1, 2, or 3;
X is defined by either one of the following two rings X1 or X2 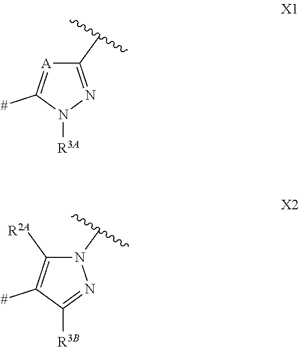
wherein
# denotes connection to the lactam moiety;
A is N or CR^{2A};
R^{2A} is H, C₁-C₆-alkyl, C₃-C₆-cycloalkyl, or C₁-C₆-alkoxy, wherein the alkyl, alkoxy and cycloalkyl moieties are unsubstituted or substituted with halogen or CN, halogen, CN, OR⁶, or NR⁶R⁷;
R^{3A} is H, C₁-C₆-alkyl, C₃-C₆-cycloalkyl, C₁-C₆-alkoxy, halogen, or CN, wherein the alkyl, alkoxy, and cycloalkyl moieties are unsubstituted or substituted with halogen, CN, or C₁-C₆-alkoxy;
R^{3B} is H, C₁-C₆-alkyl, C₃-C₆-cycloalkyl, C₁-C₆-alkoxy, wherein the alkyl, alkoxy, and cycloalkyl moieties are unsubstituted or substituted with halogen or CN;
halogen, CN, OR⁶, or NR⁶R⁷;
R⁶ and R⁷ are, identical or different, H, C₁-C₆-alkyl, C₃-C₆-cycloalkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, cycloalkyl, phenyl, and heteroaryl moieties are unsubstituted or substituted with halogen, CN, C₁-C₆-alkyl or C₁-C₆-alkoxy;
B¹ is N or CR^{B1};
B² is N or CR^{B2};
B³ is N or CR^{B3};
B⁴ is CR^{B4};
R^{B1}, R^{B2}, R^{B3}, and RB⁴ are, identical or different, H, halogen, OH, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
D is the moiety DA, DB, DC, DD, DE, or DF 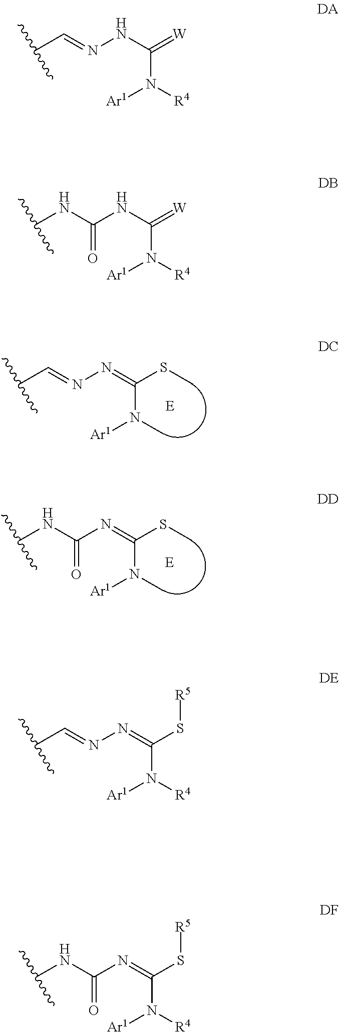
wherein
W is S or O;
R⁴ is H, C₁-C₆-alkyl, or C₃-C₆-cycloalkyl, wherein the alkyl and cycloalkyl moieties are unsubstituted or substituted with halogen or CN;
R⁵ is H, C₁-C₆-alkyl, or C₃-C₆-cycloalkyl, wherein the alkyl and cycloalkyl moieties are unsubstituted or substituted with —O—(C═O)—C₁-C₆-alkoxy, —O—(C═O)—C₁-C₆-alkyl or CN;
E is a 5- or 6-membered carbocyclic group, wherein 1 or 2 CH₂ moieties of the carbocyclic group may be replaced by a carbonyl group, O, or S, wherein the carbocyclic group is unsubstituted or substituted with R¹⁰;
Ar¹ is phenyl or 5- or 6-membered heteroaryl, which are unsubstituted or substituted with R^{Ar1}, wherein
R^{Ar1} is halogen, SF₅, OH, CN, C₁-C₆-alkyl, C₁-C₆-alkoxy, C₃-C₆-cycloalkyl, C₃-C₆-cycloalkoxy, C₁-C₆-alkyl-O—C₁-C₆-alkyl, wherein the alkyl, alkoxy, cycloalkyl, and cycloalkoxy moieties are unsubstituted or substituted with halogen or CN;
NR⁸R⁹, C═O(NR⁸R⁹), S(═O)ₘ(NR⁸R⁹), NHS(═O)ₘR⁸;
R⁸ and R⁹ are, identical or different, H, C₁-C₆-alkyl, or C₃-C₆-cycloalkyl, wherein the alkyl and cycloalkyl moieties are unsubstituted or substituted with halogen;
R¹⁰ is halogen, C₁-C₆-alkyl, or C₁-C₆-alkoxy;
and N-oxides, stereoisomers, tautomers, and agriculturally or veterinarily acceptable salts thereof.

2. The compound of formula I according to claim 1, wherein B¹ is CR^{B1}, B² is CR^{B2}, and B³ is CR^{B3}.

3. The compound of formula I according to claim 1, wherein D is DA, DB, DC, DD, or DE.

4. The compound of formula I according claim 1, wherein R^{1A}, R^{1B}, R^{1C}, and R^{1D} independently of each other are selected from H, halogen, CN, C₁-C₆-alkyl, and C₁-C₆-alkoxy, wherein the alkyl and alkoxy moieties are unsubstituted or substituted with halogen, or CN.

5. The compound of formula I according claim 1, wherein
R^{2A} is H, C₁-C₆-alkyl, C₃-C₆-cycloalkyl, or halogen, wherein the alkyl and cycloalkyl moieties are unsubstituted or substituted with halogen or CN.

6. The compound of formula I according claim 1, wherein n is 1 or 2.

7. The compound of formula I according claim 1, wherein Ar¹ is phenyl, which is unsubstituted or substituted with R^{Ar1}, and wherein
R^{Ar1} is halogen, CN, C₁-C₆-alkyl, C₁-C₆-alkoxy, or C₃-C₆-cycloalkyl, wherein the alkyl, alkoxy and cycloalkyl moieties are unsubstituted or substituted with halogen, or NR⁸R⁹, wherein R⁸ and R⁹ are C₁-C₆-alkyl.

8. The compound of formula I according to claim 1, wherein
n is 1 or 2;
R^{1A}, R^{1B}, R^{1C} and R^{1D} independently of each other are selected from H, halogen, CN, C₁-C₆-haloalkyl, and C₁-C₆-haloalkoxy;
X is X1 or X2;
A is N or CR^{2A};
R^{2A} is H, halogen, or C₁-C₆-alkyl;
R^{3A} is H, C₃-C₆-cycloalkyl, or C₁-C₆-alkyl, wherein the alkyl is unsubstituted or substituted with halogen, CN or C₁-C₆-alkoxy;
R^{3B} is H or C₁-C₆-alkyl, preferably CH₃;
B¹ is N or CR^{B1}, B² is CR^{B2}, and B³ is CR^{B3};
R^{B1}, R^{B2}, R^{B3}, and RB⁴ independently of each other are H, halogen, C₁-C₆-alkyl, or CN;
D is DA, DB, DC, DD, or DE;
W is O or S;
R⁴ is H;
R⁵ is C₁-C₆-alkyl, which is unsubstituted or substituted with —O—(C═O)—C₁-C₆-alkoxy or —O—(C═O)—C₁-C₆-alkyl;
Ar¹ is phenyl which is unsubstituted or substituted with R^{Ar1};
R^{Ar1} is halogen, CN, C₁-C₆-alkyl, C₃-C₆-cycloalkyl, C₁-C₆-alkyl-O—C₁-C₆-alkyl, or NR⁸R⁹, wherein alkyl and cycloalkyl moieties are unsubstituted or substituted with halogen;
R⁸ and R⁹ are, identical or different, H or C₁-C₆-alkyl.

9. A composition comprising a compound of formula I according to claim 1, an N-oxide or an agriculturally acceptable salt thereof, and a further active substance.

10. A method for combating or controlling an invertebrate pest comprising contacting said pest or its food supply, habitat or breeding grounds with a pesticidally effective amount of at least one compound according to claim 1.

11. A method for protecting growing plants from attack or infestation by an invertebrate pest comprising contacting a plant, or soil or water wherein the plant is growing, with a pesticidally effective amount of at least one compound according to claim 1.

12. A seed comprising a compound according to claim 1, or the enantiomers, diastereomers, or salts thereof, in an amount of from 0.1 g to 10 kg per 100 kg of seed.

13. (canceled)

14. A method for treating or protecting an animal from infestation or infection by an invertebrate pest comprising bringing the animal in contact with a pesticidally effective amount of at least one compound of the formula I according to claim 1, a stereoisomer thereof, and/or at least one veterinarily acceptable salt thereof.
